(19) United States
(12) Patent Application Publication
(10) Pub. No.: US 2020/0048267 A1
(43) Pub. Date: Feb. 13, 2020

(54) OGA INHIBITOR COMPOUNDS

(71) Applicant: Janssen Pharmaceutica NV, Beerse (BE)

(72) Inventors: José Manuel Bartolomé-Nebreda, Toledo (ES); Andrés Avelino Trabanco-Suárez, Olias del Rey (ES); Carlos Manuel Martinez Viturro, Olias del Rey (ES)

(73) Assignee: Janssen Pharmaceutica NV, Beerse (BE)

(21) Appl. No.: 16/482,327

(22) PCT Filed: Feb. 6, 2018
(86) PCT No.: PCT/EP2018/052901

§ 371 Date: Jul. 31, 2019

(30) Foreign Application Priority Data
Feb. 6, 2017 (EP) 17154751.6

(51) Int. Cl. C07D 487/10

(52) U.S. Cl. CPC C07D 487/10

(57) ABSTRACT

The present invention relates to O-GlcNAc hydrolase (OGA) inhibitors. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### FIELD OF THE INVENTION

The present invention relates to O-GlcNAc hydrolase (OGA) inhibitors, having the structure shown in Formula (I)

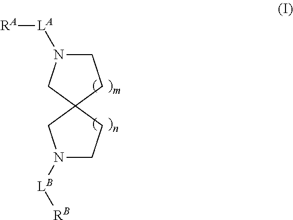

wherein the radicals are as defined in the specification. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### BACKGROUND OF THE INVENTION

O-GlcNAcylation is a reversible modification of proteins where N-acetyl-D-glucosamine residues are transferred to the hydroxyl groups of serine- and threonine residues yield O-GlcNAcylated proteins. More than 1000 of such target proteins have been identified both in the cytosol and nucleus of eukaryotes. The modification is thought to regulate a huge spectrum of cellular processes including transcription, cytoskeletal processes, cell cycle, proteasomal degradation, and receptor signaling.

O-GlcNAc transferase (OGT) and O-GlcNAc hydrolase (OGA) are the only two proteins described that add (OGT) or remove (OGA) O-GlcNAc from target proteins. OGA was initially purified in 1994 from spleen preparation and 1998 identified as antigen expressed by meningiomas and termed MGEA5, consists of 916 amino (102915 Dalton) as a monomer in the cytosolic compartment of cells. It is to be distinguished from ER- and Golgi-related glycosylation processes that are important for trafficking and secretion of proteins and different to OGA have an acidic pH optimum, whereas OGA display highest activity at neutral pH.

The OGA catalytic domain with its double aspartate catalytic center resides in then-terminal part of the enzyme which is flanked by two flexible domains. The C-terminal part consists of a putative HAT (histone acetyl transferase domain) preceded by a stalk domain. It has yet still to be proven that the HAT-domain is catalytically active.

O-GlcNAcylated proteins as well as OGT and OGA themselves are particularly abundant in the brain and neurons suggesting this modification plays an important role in the central nervous system. Indeed, studies confirmed that O-GlcNAcylation represents a key regulatory mechanism contributing to neuronal communication, memory formation and neurodegenerative disease. Moreover, it has been shown that OGT is essential for embryogenesis in several animal models and ogt null mice are embryonic lethal. OGA is also indispensable for mammalian development. Two independent studies have shown that OGA homozygous null mice do not survive beyond 24-48 hours after birth. Oga deletion has led to defects in glycogen mobilization in pups and it caused genomic instability linked cell cycle arrest in MEFs derived from homozygous knockout embryos. The heterozygous animals survived to adulthood however they exhibited alterations in both transcription and metabolism.

It is known that perturbations in O-GlcNAc cycling impact chronic metabolic diseases such as diabetes, as well as cancer. Oga heterozygosity suppressed intestinal tumorigenesis in an Apc−/+ mouse cancer model and the Oga gene (MGEA5) is a documented human diabetes susceptibility locus.

In addition, O-GlcNAc-modifications have been identified on several proteins that are involved in the development and progression of neurodegenerative diseases and a correlation between variations of O-GlcNAc levels on the formation of neurofibrillary tangle (NFT) protein by Tau in Alzheimer's disease has been suggested. In addition, O-GlcNAcylation of alpha-synuclein in Parkinson's disease has been described.

In the central nervous system six splice variants of tau have been described. Tau is encoded on chromosome 17 and consists in its longest splice variant expressed in the central nervous system of 441 amino acids. These isoforms differ by two N-terminal inserts (exon 2 and 3) and exon 10 which lie within the microtubule binding domain. Exon 10 is of considerable interest in tauopathies as it harbours multiple mutations that render tau prone to aggregation as described below. Tau protein binds to and stabilizes the neuronal microtubule cytoskeleton which is important for regulation of the intracellular transport of organelles along the axonal compartments. Thus, tau plays an important role in the formation of axons and maintenance of their integrity. In addition, a role in the physiology of dendritic spines has been suggested as well.

Tau aggregation is either one of the underlying causes for a variety of so called tauopathies like PSP (progressive supranuclear palsy), Down's syndrome (DS), FTLD (frontotemporal lobe dementia), FTDP-17 (frontotemporal dementia with Parkinsonism-17), Pick's disease (PD), CBD (corticobasal degeneration), argyrophilic grain disease (AGD), and AD (Alzheimer's disease). In addition, tau pathology accompanies additional neurodegenerative diseases like amyotrophic lateral sclerosis (ALS) or FTLD cause by C9ORF72 mutations. In these diseases, tau is post-translationally modified by excessive phosphorylation which is thought to detach tau from microtubules and makes it prone to aggregation. O-GlcNAcylation of tau regulates the extent of phosphorylation as serine or threonine residues carrying O-GlcNAc-residues are not amenable to phosphorylation. This effectively renders tau less prone to detaching from microtubules and reduces aggregation into neurotoxic tangles which ultimately lead to neurotoxicity and neuronal cell death. This mechanism may also reduce the cell-to-cell spreading of tau-aggregates released by neurons via along interconnected circuits in the brain which has recently been discussed to accelerate pathology in tau-related dementias. Indeed, hyper phosphorylated tau isolated from brains of AD-patients showed significantly reduced O-GlcNAcylation levels.

An OGA inhibitor administered to JNPL3 tau transgenic mice successfully reduced NFT formation and neuronal loss without apparent adverse effects. This observation has been confirmed in another rodent model of tauopathy where the expression of mutant tau found in FTD can be induced (tg4510). Dosing of a small molecule inhibitor of OGA was efficacious in reducing the formation of tau-aggregation and attenuated the cortical atrophy and ventricle enlargement.

Moreover, the O-GlcNAcylation of the amyloid precursor protein (APP) favours processing via the non-amyloidogenic route to produce soluble APP fragment and avoid cleavage that results in the AD associated amyloid-beta (A(3) formation.

Maintaining O-GlcNAcylation of tau by inhibition of OGA represents a potential approach to decrease tau-phosphorylation and tau-aggregation in neurodegenerative diseases mentioned above thereby attenuating or stopping the progression of neurodegenerative tauopathy-diseases.

WO2004/005293 discloses N-aryl diazaspirocyclic compounds as nicotinic receptor modulators, and particular compounds, such as 2-(6-methoxy-3-pyridazinyl)-2,7-diazaspiro[4.4]nonane, 2-(6-chloro-3-pyridinyl)-2,7-diazaspiro[4.4]nonane, 2-(5-methoxy-3-pyridinyl)-2,7-diazaspiro[4.4]nonane, 2-(3-pyridazinyl)-2,7-diazaspiro[4.4]nonane, 2-(2-pyrazinyl)-2,7-diazaspiro[4.4]nonane, 2-(5-pyrimidinyl)-2,7-diazaspiro[4.4]nonane, and 2-(3-pyridinyl)-2,7-diazaspiro[4.4]nonane; EP2301936 describes spirodiamine-diarylketoxime compounds as MCH receptor antagonists and discloses in particular 2-(4-methoxyphenyl)-2,7-diazaspiro[4.4]nonane, 2-(2-chlorophenyl)-2,7-diazaspiro[4.4]nonane, 2-(6-fluoro-3-pyridinyl)-2,6-diazaspiro[3.4]octane, 6-(6-fluoro-3-pyridinyl)-2,6-diazaspiro[3.4]octane, 2-(6-fluoro-3-pyridinyl)-2,7-diazaspiro[4.4]nonane, 2-(phenylmethyl)-2,6-diazaspiro[3.4]octane, 2-(3-chlorophenyl)-2,7-diazaspiro[4.4]nonane, 2-(phenylmethyl)-2,7-diazaspiro[4.4]nonane, 2-phenyl-2,7-diazaspiro[4.4]nonane, 2-(4-chlorophenyl)-2,7-diazaspiro[4.4]nonane, as synthetic intermediates; WO2010/108268 describes SCD inhibitor compounds, and discloses 2-(2-chlorophenyl)- and 2-(3-chlorophenyl)-2,7-diazaspiro[4.4]nonane as synthetic intermediates; WO2017/001660 describes spirobicyclic derivative compounds with antibacterial activity, and discloses 2-[4-(trifluoromethoxy)phenyl]-2,6-diazaspiro[3.4]octane and 4-(2,6-diazaspiro[3.4]oct-2-yl)-benzonitrile as intermediates; WO2010/089127 discloses spirobicyclic derivative compounds as Bradykinin receptor modulators and describes 2-(4-pyridinyl)- and 2-(phenylmethyl)-2,7-diazaspiro[4.4]nonane as intermediates; WO2013/066729 discloses pyrimidinone derivatives as IRAK inhibitors, and describes 2-(2-pyrimidinyl)-2,7-diazaspiro[4.4]nonane [1450891-68-5] and 2-[(3-chloro-2-fluorophenyl)methyl]-2,7-diazaspiro[4.4]nonane (free base and hydrochloride salt) as synthetic intermediates; WO2014/023723 discloses 6-[4-(trifluoromethyl)phenyl]-2,6-diazaspiro[3.4]octane [1609025-57-1] as an intermediate; Sippy et al. Bioorg. Med. Chem. Lett. 2009, 19(6), 1682-1685 describes N-(3-pyridinyl)-spirocyclic diamines and their affinity to nACh receptors. 2-(phenylmethyl)-2,7-diazaspiro[4.4]nonane is disclosed as an intermediate and 2-(6-chloro-3-pyridinyl)- and 2-(3-pyridinyl)-2,7-diazaspiro[4.4]nonane were found to have weak binding affinity; Orain et al. Synlett, 26(13), 1815-1818 concerns the synthesis of protected spirocyclic diamine scaffolds, such as 6-(phenylmethyl)-2,6-diazaspiro[3.4]octane [135380-28-8 ]; Weinberg et al. Tetrahedron 2013, 69(23), 4694-4707 describes the synthesis of spirocyclic diamine scaffolds, a particular example of which is 2-(6-chloro-3-pyridinyl)-2,7-diazaspiro[4.4]nonane [646056-57-7]. Trapannone et al. Biochem. Soc. T. 2016, 44(1), 88-93 comprises a review on O-GlcNAc hydrolase inhibitors.

The following compounds are commercially available: 2-[(3-chloro-2-fluorophenyl)methyl]-2,7-diazaspiro[4.4]nonane hydrochloride (1:1), 2-[[4-(methylthio)phenyl]methyl]-2,7-diazaspiro[4.4]nonane hydrochloride (1:1), 2-[(3-chloro-4-pyridinyl)methyl]-2,7-diazaspiro[4.4]nonane hydrochloride (1:2), 2-[(2-chloro-5-fluorophenyl)methyl]-2,7-diazaspiro[4.4]nonane hydrochloride (1:1), 2-[(2-chloro-5-methoxyphenyl)methyl]-2,7-diazaspiro[4.4]nonane hydrochloride (1:1), 2-[(4-ethoxyphenyl)methyl]-2,7-diazaspiro[4.4]nonane hydrochloride (1:1), 2-[(4-bromophenyl)methyl]-2,7-diazaspiro[4.4]nonane hydrochloride (1:1), N-[2-(2,7-diazaspiro[4.4]non-2-ylmethyl)phenyl]-acetamide hydrochloride (1:1), 2-[1-(3-fluorophenyl)ethyl]-2,7-diazaspiro[4.4]nonane hydrochloride (1:1), 2-[(5-methyl-2-pyridinyl)methyl]-2,7-diazaspiro[4.4]nonane hydrochloride (1:1), 2-[1-(2,5-difluorophenyl)ethyl]-2,7-diazaspiro[4.4]nonane hydrochloride (1:1), 2-[(2-bromo-4-methylphenyl)methyl]-2,7-diazaspiro[4.4]nonane hydrochloride (1:1), 2-[(4-chloro-2-methylphenyl)methyl]-2,7-diazaspiro[4.4]nonane hydrochloride (1:1), 2-[(3-chloro-5-ethoxy-4-propoxyphenyl)methyl]-2,7-diazaspiro[4.4]nonane hydrochloride (1:1), 2-[(4-bromo-2-chlorophenyl)methyl]-2,7-diazaspiro[4.4]nonane hydrochloride (1:1), 2-[(4-bromo-2-methylphenyl)methyl]-2,7-diazaspiro[4.4]nonane hydrochloride (1:1), 5-(2,7-diazaspiro[4.4]non-2-ylmethyl)-N-methyl-N-(1-methylethyl)-2-pyridinamine hydrochloride (1:1), 2-[(4-chlorophenyl)methyl]-2,7-diazaspiro[4.4]nonane, hydrochloride (1:1), 2-[(3-bromo-4-ethoxy-5-methoxyphenyl)methyl]-2,7-diazaspiro[4.4]nonane hydrochloride (1:1), 2[4-(trifluoromethyl)-2-pyridinyl]-2,7-diazaspiro[4.4]nonane, and 2-[1-(3-methylphenyl)ethyl]-2,7-diazaspiro[4.4]nonane hydrochloride (1:1).

There is still a need for OGA inhibitors with an advantageous balance of properties, for example with improved potency, better selectivity, brain penetration and/or better side effect profile. It has now been found that compounds according to the present invention exhibit OGA inhibitory activity and a good balance of properties.

### SUMMARY OF THE INVENTION

The present invention concerns spirobicyclic compounds of Formula (I)

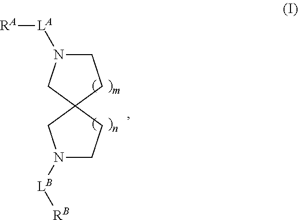

and the stereoisomeric forms thereof, wherein

m and n each independently represent 0 or 1, with the proviso that they are not both simultaneously 0;

L^{A} is a covalent bond or CHR; wherein

R is hydrogen or C₁₋₄alkyl optionally substituted with 1, 2 or 3 independently selected halo substituents;

R^{A} represents a 6-membered aryl or heteroaryl radical selected from the group consisting of phenyl, pyridin-2-yl, pyridin-3-yl, pyridin-4-yl, pyridazin-3-yl, pyrimidin-2-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2 or 3 independently selected halo substituents; C₃₋₇cycloalkyl; C₁₋₄alkyloxy optionally substituted with 1, 2 or 3 independently selected halo substituents; and NR^{a}R^{aa}, wherein R^{a} is hydrogen or C₁₋₄alkyl optionally substituted with 1, 2, or 3 independently selected halo substituents, and R^{aa} is selected from the group consisting of hydrogen, C₁₋₄alkyl optionally substituted with 1, 2, or 3 independently selected halo substituents, and —C(═O)C₁₋₄alkyl;

L^{B} is CHR¹; wherein R¹ is hydrogen or C₁₋₄alkyl optionally substituted with 1, 2 or 3 independently selected halo substituents; and

R^{B} represents a heterocyclic ring or ring system selected from the group consisting of (b-1), (b-2), (b-3), (b-4), (b-5), (b-6), (b-7), (b-8), (b-9), (b-10), (b-11) and (b-12):

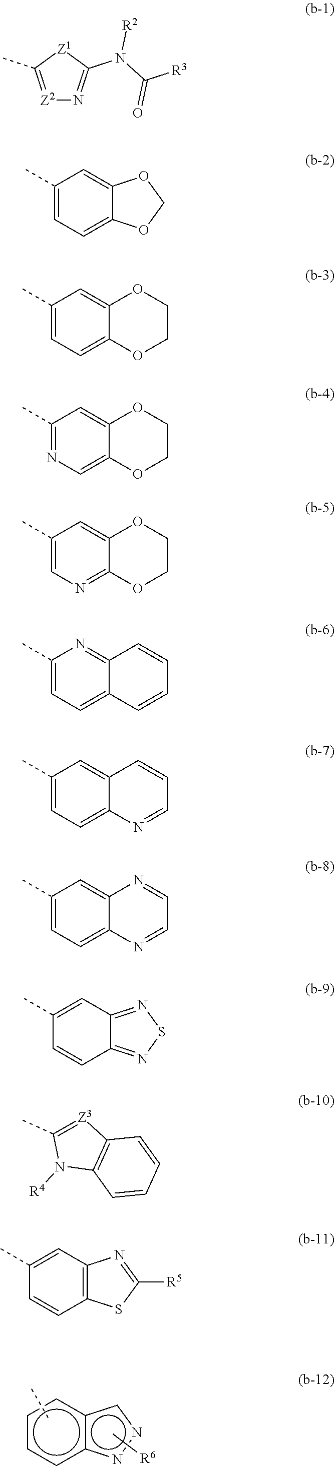

wherein

Z¹ is O, NR^{1z} or S; wherein R^{1z} is hydrogen or C₁₋₄alkyl;

Z² and Z³ each independently represent CH or N;

R³ is C₁₋₄alkyl;

R², R⁴, R⁵ and R⁶ each independently represent hydrogen or C₁₋₄alkyl; or

-L^{B}-R^{B} is a radical of formula (b-13)

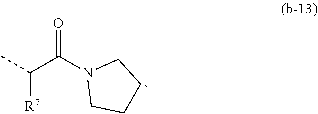

wherein R⁷ is hydrogen or C₁₋₄alkyl;

and the pharmaceutically acceptable salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of preventing or treating a disorder mediated by the inhibition of O-GlcNAc hydrolase (OGA), comprising administering to a subject in need thereof a prophylactically or a therapeutically effective amount of any of the compounds or pharmaceutical compositions described above.

Further exemplifying the invention are methods of inhibiting OGA, comprising administering to a subject in need thereof a prophylactically or therapeutically effective amount of any of the compounds or pharmaceutical compositions described above.

An example of the invention is a method of preventing or treating a disorder selected from a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and argyrophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of any of the compounds or pharmaceutical compositions described above.

Another example of the invention is any of the compounds described above for use in preventing or treating a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and argyrophilic grain disease; or a neurodegenerative disease accompanied by a tau pathology, in particular a neurodegenerative disease selected from amyotrophic lateral sclerosis or frontotemporal lobe dementia caused by C9ORF72 mutations, in a subject in need thereof.

### DETAILED DESCRIPTION OF THE INVENTION

The present invention is directed to compounds of Formula (I) as defined herein before, and pharmaceutically acceptable addition salts and solvates thereof. The compounds of Formula (I) are inhibitors of O-GlcNAc hydrolase (OGA) and may be useful in the prevention or treatment of tauopathies, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and argyrophilic grain disease; or may be useful in the prevention or treatment of neurodegenerative diseases accompanied by a tau pathology, in particular a neurodegenerative disease selected from amyotrophic lateral sclerosis or frontotemporal lobe dementia caused by C9ORF72 mutations.

In a particular embodiment, the invention is directed to compounds of Formula (I) as referred to herein, and the tautomers and the stereoisomeric forms thereof, wherein m is 1 and n is 0 or 1, in particular, m and n are 1;

and the pharmaceutically acceptable salts and the solvates thereof.

In a further embodiment, the invention is directed to compounds of Formula (I) as referred to herein, and the tautomers and the stereoisomeric forms thereof, wherein m is 0 and n is 1;

and the pharmaceutically acceptable salts and the solvates thereof.

In a further embodiment, the invention is directed to compounds of Formula (I) as referred to herein, and the tautomers and the stereoisomeric forms thereof, wherein L^{A} is a covalent bond;

and the pharmaceutically acceptable salts and the solvates thereof.

In a further embodiment, the invention is directed to compounds of Formula (I) as referred to herein, and the tautomers and the stereoisomeric forms thereof, wherein L^{A} is CHR; and the pharmaceutically acceptable salts and the solvates thereof.

In a further embodiment, the invention is directed to compounds of Formula (I) as referred to herein, and the tautomers and the stereoisomeric forms thereof, wherein R^{A} is pyridin-4-yl, pyrimidin-4-yl or pyrazin-2-yl each of which is optionally substituted with 1 or 2 substituents each independently selected from the group consisting of

C₁₋₄alkyl and C₃₋₇cycloalkyl, and the pharmaceutically acceptable salts and the solvates thereof.

In a further embodiment, the invention is directed to compounds of Formula (I) as referred to herein, and the tautomers and the stereoisomeric forms thereof, wherein R^{A} is pyridin-4-yl or pyrazin-2-yl, each of which is optionally substituted with 1 or 2 substituents each independently selected from the group consisting of C₁₋₄alkyl and

C₃₋₇cycloalkyl; and the pharmaceutically acceptable salts and the solvates thereof.

In a further embodiment, the invention is directed to compounds of Formula (I) as referred to herein, and the tautomers and the stereoisomeric forms thereof, wherein R^{A} is pyridin-4-yl, pyridin-3-yl or pyridin-2-yl each substituted with 1 or 2 substituents each independently selected from the group consisting of C₁₋₂alkyl and C₁₋₂alkyloxy.

In a further embodiment, the invention is directed to compounds of Formula (I) as referred to herein, and the tautomers and the stereoisomeric forms thereof, wherein L^{A} is a bond; and the pharmaceutically acceptable salts and the solvates thereof.

In a further embodiment, the invention is directed to compounds of Formula (I) as referred to herein, and the tautomers and the stereoisomeric forms thereof, wherein L^{B} is CH₂ or CH(CH₃) and R^{B} is a radical of formula (b-1), (b-2), (b-3), (b-8), (b-11) or (b-12);

and the pharmaceutically acceptable salts and the solvates thereof.

In a further embodiment, the invention is directed to compounds of Formula (I) as referred to herein, and the tautomers and the stereoisomeric forms thereof, wherein L^{B} is CH₂ or CH(CH₃) and R^{B} is a radical of formula (b-1) or (b-8);

and the pharmaceutically acceptable salts and the solvates thereof.

In a further embodiment, the invention is directed to compounds of Formula (I) as referred to herein, and the tautomers and the stereoisomeric forms thereof, wherein L^{B} is CH₂ or CH(CH₃) and R^{B} is a radical of formula (b-1), wherein Z¹ is 0, Z² is CH, R³ is C₁₋₄alkyl and R² is hydrogen;

and the pharmaceutically acceptable salts and the solvates thereof.

In a further embodiment, the invention is directed to compounds of Formula (I) as referred to herein, and the tautomers and the stereoisomeric forms thereof, wherein

L^{A} is a covalent bond;

R^{A} is pyridin-4-yl, pyridin-3-yl or pyridin-2-yl each substituted with 1 or 2 substituents each independently selected from the group consisting of C₁₋₂alkyl and C₁₋₂alkyloxy;

L^{B} is CH₂ or CH(CH₃);

and R^{B} is a radical of formula (b-1), wherein Z¹ is O, Z² is CH, R³ is C₁₋₄alkyl and R² is hydrogen; and the pharmaceutically acceptable salts and the solvates thereof.

Compounds of Formula (II) as described herein

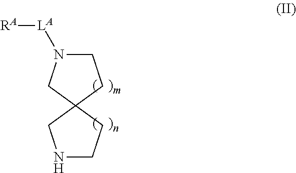

and the stereoisomers thereof, wherein all variables are as described with respect to compounds of formula (I), are useful as synthetic intermediates and additionally, some of them display OGA inhibitory activity. Therefore, in an additional aspect, the invention relates to compounds of Formula (II), and the stereoisomeric forms thereof, and the pharmaceutically acceptable salts and the solvates thereof. In a further aspect, the invention relates to compounds of Formula (II), and the stereoisomeric forms thereof, and the pharmaceutically acceptable salts and the solvates thereof for use as an OGA inhibitor as a medicament, in particular for use in the treatment of tauopathies, as described herein.

Definitions

“Halo” shall denote fluoro, chloro and bromo; “C₁₋₄alkyl” shall denote a straight or branched saturated alkyl group having 1, 2, 3 or 4 carbon atoms, respectively e.g. methyl, ethyl, 1-propyl, 2-propyl, butyl, 1-methyl-propyl, 2-methyl-1-propyl, 1,1-dimethylethyl, and the like; “C₁₋₄alkyloxy” shall denote an ether radical wherein C₁₋₄alkyl is as defined before.

The term “subject” as used herein, refers to an animal, preferably a mammal, most preferably a human, who is or has been the object of treatment, observation or experiment.

As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. The term “prophylactically effective amount” as used herein, means that amount of active compound or pharmaceutical agent that substantially reduces the potential for onset of the disease or disorder being prevented.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid,

p-toluenesulfonic acid, trifluoromethylsulfonic acid, and undecylenic acid. Representative bases which may be used in the preparation of pharmaceutically acceptable addition salts include, but are not limited to, the following: ammonia,

L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

### Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

Experimental Procedure 1

Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

Experimental Procedure 1

Final compounds of Formula (I), wherein L^{B} is CHR¹, herein referred to as (I-a), can be prepared by reacting an intermediate compound of Formula (II) with a carbonyl compound of Formula (VI) according to reaction scheme (1). The reaction is performed in a suitable reaction-inert solvent, such as, for example, dichloromethane, a metal hydride, such as, for example sodium triacetoxyborohydride, sodium cyanoborohydride or sodium borohydride and may require the presence of a suitable base, such as, for example, triethylamine, and/or a Lewis acid, such as, for example titanium tetraisopropoxide or titanium tetrachloride, under thermal conditions, such as, 0° C. or room temperature, or 140° C., for example for 1 hour or 24 hours. In reaction scheme (1) all variables are defined as in Formula (I).

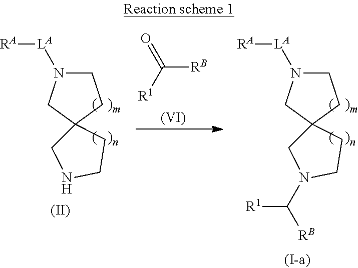

Experimental Procedure 2

Final compounds of Formula (I), wherein L^{B} is CHR¹ and L^{A} is a covalent bond, herein referred to as (I-b), can be prepared by reacting an intermediate compound of Formula (III) with a compound of Formula (VII) according to reaction scheme (2). The reaction is performed in a suitable reaction-inert solvent, such as, for example, isopropanol or acetonitrile, a suitable base, such as, for example, trimethylamine under thermal conditions, such as, 100-150° C., for example for 1 hour or 24 hours. In reaction scheme (2) all variables are defined as in Formula (I).

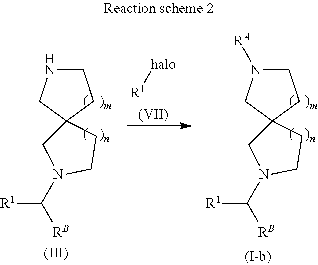

Experimental Procedure 3

Final compounds of Formula (I) wherein L^{B} is CHR¹, herein referred to as (I-c), can be prepared by reacting an intermediate compound of Formula (II) with a compound of Formula (VIII) followed by reaction of the formed imine derivative with an intermediate compound of Formula (IX) according to reaction scheme (3). The reaction is performed in a suitable reaction-inert solvent, such as, for example, anhydrous dichloromethane, a Lewis acid, such as, for example titanium tetraisopropoxide or titanium tetrachloride, under thermal conditions, such as, 0° C. or room temperature, for example for 1 hour or 24 hours. In reaction scheme (3) all variables are defined as in Formula (I), le is C₁₋₄alkyl, and halo is chloro, bromo or iodo

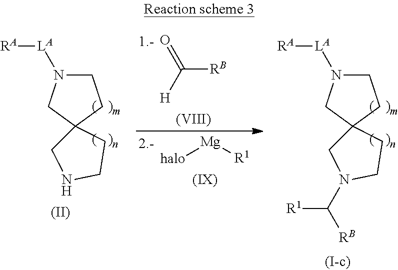

Experimental Procedure 4

Intermediate compounds of Formula (II) can be prepared cleaving a protecting group in an intermediate compound of Formula (IV) according to reaction scheme (4). In reaction scheme (4) all variables are defined as in Formula (I), and PG is a suitable protecting group of the nitrogen function such as, for example, tert-butoxycarbonyl (Boc), ethoxycarbonyl, benzyl, benzyloxycarbonyl (Cbz). Suitable methods for removing such protecting groups are widely known by the person skilled in the art and comprise but are not limited to: Boc deprotection: treatment with a protic acid, such as, for example, trifluoroacetic acid, in a reaction inert solvent, such as, for example, dichloromethane; ethoxycarbonyl deprotection: treatment with a strong base, such as, for example, sodium hydroxide, in a reaction inert solvent such as for example wet tetrahydrofuran; benzyl deprotection: catalytic hydrogenation in the presence of a suitable catalyst, such as, for example, palladium on carbon, in a reaction inert solvent, such as, for example, ethanol; benzyloxycarbonyl deprotection: catalytic hydrogenation in the presence of a suitable catalyst, such as, for example, palladium on carbon, in a reaction inert solvent, such as, for example, ethanol.

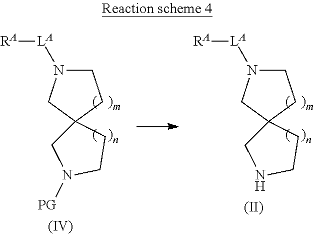

Experimental Procedure 5

Intermediate compounds of Formula (IV-a) can be prepared by reaction of an intermediate compound of Formula (V) with a carbonyl compound of Formula (X) according to reaction scheme (5). The reaction is performed in a suitable reaction-inert solvent, such as, for example, dichloromethane, a metal hydride, such as, for example sodium triacetoxyborohydride, sodium cyanoborohydride or sodium borohydride and may require the presence of a suitable base, such as, for example, triethylamine, and/or a Lewis acid, such as, for example titanium tetraisopropoxide or titanium tetrachloride, under thermal conditions, such as, 0° C. or room temperature, or 140° C., for example for 1 hour or 24 hours. In reaction scheme (5) all variables are defined as in Formula (I), L^{A} is CHR and PG is a suitable protecting group of the nitrogen function such as, for example, tert-butoxycarbonyl (Boc), ethoxycarbonyl, benzyl, benzyloxycarbonyl (Cbz).

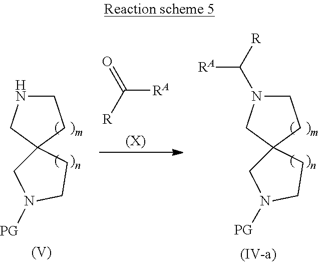

Experimental Procedure 6

Intermediate compounds of Formula (IV-b) can be prepared by reacting an intermediate compound of Formula (V) with a compound of Formula (XI) according to reaction scheme (6). The reaction is performed in a suitable reaction-inert solvent, such as, for example, isopropanol or acetonitrile, a suitable base, such as, for example, trimethylamine under thermal conditions, such as, 100-150° C., for example for 1 hour or 24 hours. In reaction scheme (6) all variables are defined as in Formula (I) and L^{A} is a bond.

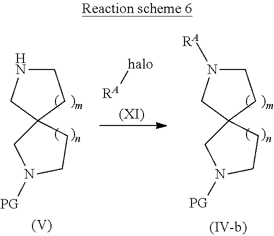

Experimental Procedure 7

Intermediate compounds of Formula (IV-c) can be prepared by “Suzuki coupling” reaction of an intermediate compound of Formula (IV-b′) with a compound of Formula (XII) according to reaction scheme (7). The reaction is performed in a suitable reaction-inert solvent, such as, for example, 1,4-dioxane, and a suitable catalyst, such as, for example, tetrakis(triphenylphosphine)palladium (0), a suitable base, such as, for example, Na₂CO₃ (aq. sat. soltn.), under thermal conditions, such as, for example, 150° C., for example for 15 min under microwave irradiation. In reaction scheme (7) all variables are defined as in Formula (I) wherein and L^{A} is a bond, R^{A} is a pyrazyl radical substituted with C₁₋₄alkyl, halo is chloro, bromo or iodo and Alk is C₁₋₄alkyl.

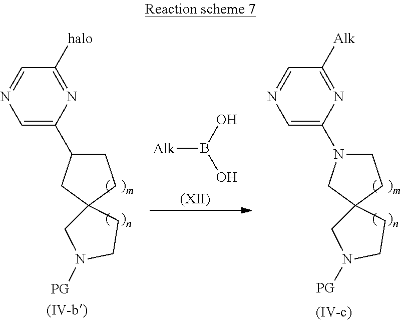

Experimental Procedure 8

Intermediate compounds of Formula (IV-d) can be prepared by hydrogenation reaction of an intermediate compound of Formula (IV-b′) according to reaction scheme (8) The reaction is performed in a suitable reaction-inert solvent, such as, for example, ethanol, and a suitable catalyst, such as, for example 10% palladium (0) on carbon in the presence of hydrogen, under thermal conditions, such as, for example, 50° C., for example for 1 min in a H-cube reactor. In reaction scheme (8) all variables are defined as in Formula (I) wherein L^{A} is a bond, R^{A} is a halopyrazyl radical, and halo is chloro, bromo or iodo.

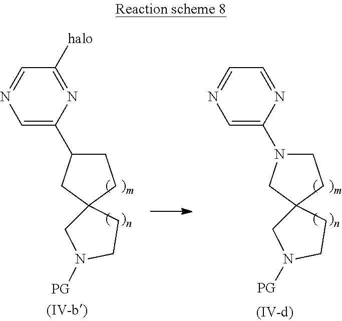

Experimental Procedure 9

Intermediate compounds of Formula (III) can be prepared by reacting an intermediate compound of Formula (V) with a compound of Formula (VI) according to reaction scheme (9). The reaction is performed in a suitable reaction-inert solvent, such as, for example, dichloromethane, a metal hydride, such as, for example sodium triacetoxyborohydride, sodium cyanoborohydride or sodium borohydride and may require the presence of a suitable base, such as, for example, triethylamine, and/or a Lewis acid, such as, for example titanium tetraisopropoxide or titanium tetrachloride, under thermal conditions, such as, 0° C. or room temperature, or 140° C., for example for 1 hour or 24 hours. In reaction scheme (9) all variables are defined as in Formula (I).

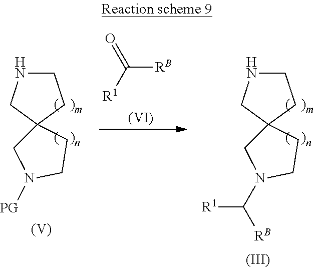

Intermediates of Formula, (V), (VI), (VII) (VIII), (IX), (X), (XI) and (XII) are commercially available or can be prepared by know procedures to those skilled in the art.

Pharmacology

The compounds of the present invention and the pharmaceutically acceptable compositions thereof inhibit O-GlcNAc hydrolase (OGA) and therefore may be useful in the treatment or prevention of diseases involving tau pathology, also known as tauopathies, and diseases with tau inclusions. Such diseases include, but are not limited to Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease,

Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

As used herein, the term “treatment” is intended to refer to all processes, wherein there may be a slowing, interrupting, arresting or stopping of the progression of a disease or an alleviation of symptoms, but does not necessarily indicate a total elimination of all symptoms. As used herein, the term “prevention” is intended to refer to all processes, wherein there may be a slowing, interrupting, arresting or stopping of the onset of a disease.

The invention also relates to a compound according to the general Formula (I), a stereoisomeric form thereof or a pharmaceutically acceptable acid or base addition salt thereof, for use in the treatment or prevention of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

The invention also relates to a compound according to the general Formula (I), a stereoisomeric form thereof or a pharmaceutically acceptable acid or base addition salt thereof, for use in the treatment, prevention, amelioration, control or reduction of the risk of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

In particular, the diseases or conditions may in particular be selected from a tauopathy, more in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and argyrophilic grain disease; or the diseases or conditions may in particular be neurodegenerative diseases accompanied by a tau pathology, more in particular a neurodegenerative disease selected from amyotrophic lateral sclerosis or frontotemporal lobe dementia caused by C9ORF72 mutations.

Preclinical states in Alzheimer's and Tauopathy Diseases:

In recent years the United States (US) National Institute for Aging and the International Working Group have proposed guidelines to better define the preclinical (asymptomatic) stages of AD (Dubois B, et al. Lancet Neurol. 2014; 13:614-629; Sperling, R A, et al. Alzheimers Dement. 2011; 7:280-292). Hypothetical models postulate that Aβ accumulation and tau-aggregation begins many years before the onset of overt clinical impairment. The key risk factors for elevated amyloid accumulation, tau-aggregation and development of AD are age (i.e., 65 years or older), APOE genotype, and family history. Approximately one third of clinically normal older individuals over 75 years of age demonstrate evidence of AP or tau accumulation on PET amyloid and tau imaging studies, the latter being less advanced currently. In addition, reduced Abeta-levels in CSF measurements are observed, whereas levels of non-modified as well as phosphorylated tau are elevated in CSF. Similar findings are seen in large autopsy studies and it has been shown that tau aggregates are detected in the brain as early as 20 years of age and younger.

Amyloid-positive (Aβ+) clinically normal individuals consistently demonstrate evidence of an “AD-like endophenotype” on other biomarkers, including disrupted functional network activity in both functional magnetic resonance imaging (MRI) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases Aβ production or the aggregation of tau is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease or tauopathies. Cognitively normal individuals with amyloid beta or tau aggregation evident on PET scans, or changes in CSF Abeta, tau and phospho-tau are defined as being in an “asymptomatic at-risk state for Alzheimer's disease (AR-AD)” or in a “asymptomatic state of tauopathy”. Individuals with a fully penetrant dominant autosomal mutation for familial Alzheimer's disease are said to have “presymptomatic Alzheimer's disease”. Dominant autosomal mutations within the tau-protein have been described for multiple forms of tauopathies as well.

Thus, in an embodiment, the invention also relates to a compound according to the general Formula (I′) or (I), a stereoisomeric form thereof or a pharmaceutically acceptable acid or base addition salt thereof, for use in control or reduction of the risk of preclinical Alzheimer's disease, prodromal Alzheimer's disease, or tau-related neurodegeneration as observed in different forms of tauopathies. As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a prophylactically or a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a prophylactically or a therapeutically effective amount of a compound according to the invention to a subject in need thereof.

The invention also relates to a method for modulating O-GlcNAc hydrolase (OGA) activity, comprising administering to a subject in need thereof, a prophylactically or a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment, the compounds according to the invention are preferably formulated prior to administration. As described herein below, suitable pharmaceutical formulations are prepared by known procedures using well known and readily available ingredients.

The compounds of the present invention, that can be suitable to treat or prevent any of the disorders mentioned above or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agent in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™) of the American Psychiatric Association utilizes terms such as neurocognitive disorders (NCDs) (both major and mild), in particular, neurocognitive disorders due to Alzheimer's disease. Such terms may be used as an alternative nomenclature for some of the diseases or conditions referred to herein by the skilled person.

Pharmaceutical Compositions

The present invention also provides compositions for preventing or treating diseases in which inhibition of O-GlcNAc hydrolase (OGA) is beneficial, such as Alzheimer's disease, progressive supranuclear palsy, Down's syndrome, frontotemporal lobe dementia, frontotemporal dementia with Parkinsonism-17, Pick's disease, corticobasal degeneration, argyrophilic grain disease, amyotrophic lateral sclerosis or frontotemporal lobe dementia caused by C9ORF72 mutations, said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets.

Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The invention also provides a kit comprising a compound according to the invention, prescribing information also known as “leaflet”, a blister package or bottle, and a container. Furthermore, the invention provides a kit comprising a pharmaceutical composition according to the invention, prescribing information also known as “leaflet”, a blister package or bottle, and a container. The prescribing information preferably includes advice or instructions to a patient regarding the administration of the compound or the pharmaceutical composition according to the invention. In particular, the prescribing information includes advice or instruction to a patient regarding the administration of said compound or pharmaceutical composition according to the invention, on how the compound or the pharmaceutical composition according to the invention is to be used, for the prevention and/or treatment of a tauopathy in a subject in need thereof. Thus, in an embodiment, the invention provides a kit of parts comprising a compound of Formula (I) or a stereoisomeric for thereof, or a pharmaceutically acceptable salt or a solvate thereof, or a pharmaceutical composition comprising said compound, and instructions for preventing or treating a tauopathy. The kit referred to herein can be, in particular, a pharmaceutical package suitable for commercial sale.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

Experimental Part

Hereinafter, the term “m.p.” means melting point, “min” means minutes, “ACN” means acetonitrile, “aq.” means aqueous, “Boc” means tert butyloxycarbonyl, “DMF” means dimethylformamide, “r.t.” or “RT” means room temperature, “rac” or “RS” means racemic, “sat.” means saturated, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “ⁱPrOH” means isopropyl alcohol, “RP” means reversed phase, “Rₜ” means retention time (in minutes), “[M+H]⁺” means the protonated mass of the free base of the compound, “wt” means weight, “THF” means tetrahydrofuran, “EtOAc” means ethyl acetate, “DCM” means dichloromethane, “MeOH” means methanol, “soltn” or “sol.” means solution, “EtOH” means ethanol, and Pd(OAc)₂ means Palladium(II) acetate.

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centres has been designated as “*R” or “*S” when the absolute stereochemistry is undetermined although the compound itself has been isolated as a single stereoisomer and is enantiomerically/diastereomerically pure. The enantiomeric excess of compounds reported herein was determined by analysis of the racemic mixture by supercritical fluid chromatography (SFC) followed by SFC comparison of the separated enantiomer(s).

Microwave assisted reactions were performed in a single-mode reactor: Initiator™ Sixty EXP microwave reactor (Biotage AB), or in a multimode reactor: MicroSYNTH Labstation (Milestone, Inc.).

Thin layer chromatography (TLC) was carried out on silica gel 60 F254 plates (Merck) using reagent grade solvents. Open column chromatography was performed on silica gel, particle size 60 Å, mesh=230-400 (Merck) using standard techniques.

Automated flash column chromatography was performed using ready-to-connect cartridges, on irregular silica gel, particle size 15-40 μm (normal phase disposable flash columns) on different flash systems: either a SPOT or LAFLASH systems from Armen Instrument, or PuriFlash® 430evo systems from Interchim, or 971-FP systems from Agilent, or Isolera 1SV systems from Biotage.

### A. PREPARATION OF THE INTERMEDIATES

Preparation of Intermediate 1

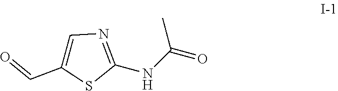

Acetyl chloride (6 mL, 84.38 mmol) was added to a solution of 2-amino-5-formylthiazole (10 g, 78 mmol) and diisopropylamine (45 mL, 261.1 mmol) in DCM (100 mL) at 0° C. The resulting mixture was allowed to warm to rt and further stirred at rt for 17 h. NH₄Cl (aq. sat. soltn.) was added and the mixture was extracted with EtOAc. The organic layer was separated, dried over MgSO₄, filtered and concentrated in vacuo. The residue thus obtained was purified by flash column chromatography (silica; dry load, EtOAc in DCM 0/100 to 50/50) and the desired fractions were concentrated in vacuo to yield Intermediate 1 as yellow solid (8.6 g, 65% yield).

Preparation of Intermediate 2, 2a and 2b

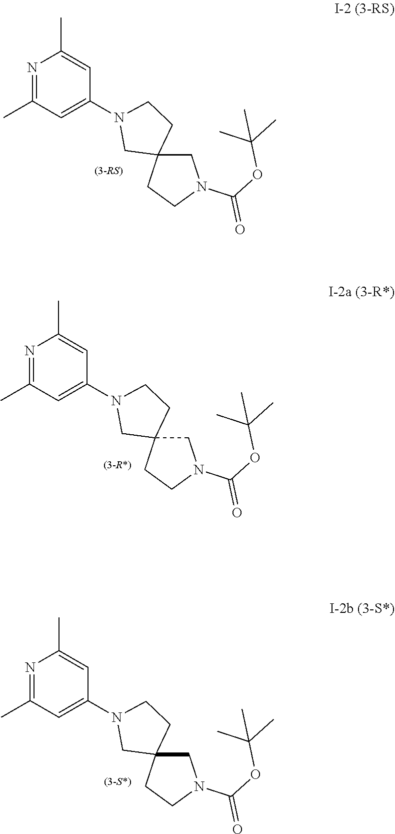

A mixture of 2-Boc-2,7-diazaspiro[4.4]nonane (CAS: 236406-49-8; 100 mg, 0.442 mmol), 4-chloro-2,6-dimethylpyridine (75.1 mg, 0.53 mmol) and diisopropylethylamine (0.152 mL, 0.88 mmol) in isopropanol (1.5 mL) was first stirred at 120° C. for 30 min into a sealed tube and then at 150° C. under microwave irradiation for 90 min. Then the solvent was evaporated in vacuo and the residue thus obtained was taken up in EtOAc and washed with NaHCO₃ (aq. sat. soltn). The organic layer was separated, dried over MgSO₄, filtered and concentrated in vacuo. The residue thus obtained was purified by flash column chromatography (silica gel, MeOH in DCM 0/100 to 15/85) and the desired fractions were concentrated in vacuo to yield Intermediate 2 (78 mg; 53% yield) as a colorless syrup.

Intermediate 2 (3.43 g) was subjected to preparative HPLC (Stationary phase: Chiralpak AD-H 5 μm 250*30 mm, Mobile phase: 78% CO₂, 22% mixture of EtOH/iPrOH 50/50 v/v (+5% iPrNH₂)) to give Intermediate 2a (1.61 g) and Intermediate 2b (1.78 g).

Preparation of Intermediate 3

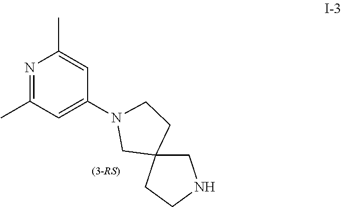 [SMILES: Cc1cc(N2CCC3(CCNC3)C2)cc(C)n1]

HCl (0.59 mL, 4M solution in 1,4-dioxane) was added to a solution of Intermediate 2 (78 mg, 0.24 mmol) in 1,4-dioxane (1.24 mL) at rt. The mixture was stirred at rt for 16 h. The volatiles were evaporated under vacuo and the residue thus obtained was triturated with EtOAc to yield Intermediate 3 (57 mg; 80% yield; bis-HCl salt) as brownish solid.

Preparation of Intermediate 3a

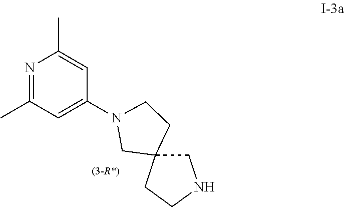

HCl (2.5 mL, 4M solution in 1,4-dioxane) was added to a solution of Intermediate 2a (0.32 g, 0.97 mmol) in 1,4-dioxane (5 mL) at rt and under N₂ atmosphere. The mixture was stirred at rt for 16 h. The volatiles were evaporated under vacuum affording a residue that was taken up in MeOH and passed through an isolute SCX-2 cartridge. The product was eluted with a 7N solution of NH₃ in MeOH. The volatiles were evaporated in vacuo affording Intermediate 3a (0.23 g, quantitative) as a pale yellow oil.

Preparation of Intermediate 3b

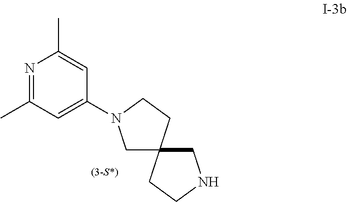

HCl (2.5 mL, 4M solution in 1,4-dioxane) was added to a solution of Intermediate 2b (0.38 g, 0.97 mmol) in 1,4-dioxane (5 mL) at rt and under N₂ atmosphere. The mixture was stirred at rt for 16 h. The volatiles were evaporated under vacuum affording a residue that was taken up in MeOH and passed through an isolute SCX-2 cartridge. The product was eluted with a 7N solution of NH₃ in MeOH. The volatiles were evaporated in vacuo affording Intermediate 3b (0.23 g, 88% yield) as a pale yellow oil.

Preparation of Intermediate 4

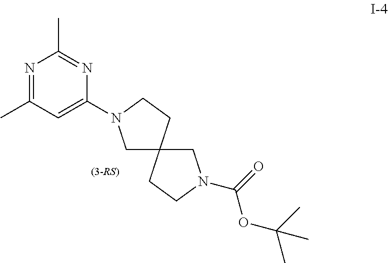 [SMILES: Cc1cc(N2CCC3(CCN(C(=O)OC(C)(C)C)C3)C2)nc(C)n1]

A mixture of 2-Boc-2,7-diazaspiro[4.4]nonane (CAS: 236406-49-8; 250 mg, 1.05 mmol), 4-chloro-2,6-dimethylpyrimidine (189 mg, 1.33 mmol) and diisopropylethylamine (0.38 mL, 2.21 mmol) in isopropanol (3.75 mL) was first stirred at 120° C. for 30 min into a sealed tube and then at 150° C. under microwave irradiation for 90 min. Then the solvent was evaporated in vacuo and the residue thus obtained was taken up in EtOAc and washed with NaHCO₃ (aq. sat. soltn). The organic layer was separated, dried over MgSO₄, filtered and concentrated in vacuo. The residue thus obtained was purified by flash column chromatography (silica gel, MeOH in DCM 0/100 to 15/85) and the desired fractions were concentrated in vacuo to yield Intermediate 4 (265 mg; 72% yield) as a colorless syrup.

Preparation of Intermediate 5

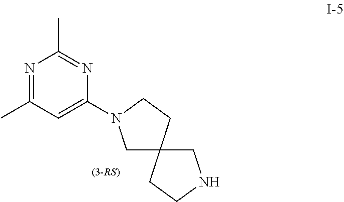 [SMILES: Cc1cc(N2CCC3(CCNC3)C2)nc(C)n1]

HCl (2 mL, 4M solution in 1,4-dioxane) was added to a solution of Intermediate 4 (265 mg, 0.24 mmol) in 1,4-dioxane (1.24 mL) at rt. The mixture was stirred at rt for 16 h. The volatiles were evaporated under vacuo to yield Intermediate 5 (214 mg, quantitative, HCl salt) as brownish solid.

Preparation Of Intermediate 6

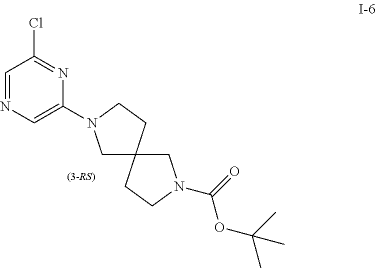 [SMILES: CC(C)(C)OC(=O)N1CCC2(CCN(c3cncc(Cl)n3)C2)C1]

Diisopropylethylamine (1.56 mL, 9.03 mmol) was added to a stirred solution of 2-Boc-2,7-diazaspiro[4.4]nonane (CAS: 236406-49-8; 1.52 g, 6.72 mmol), and 2,6-dichloropyrazine (1.35 g, 9 mmol) in acetonitrile (13.3 mL) under N₂ atmosphere. The mixture was stirred at 150° C. under microwave irradiation for 15 min. Then NH₄Cl (aq. sat. soltn.) was added and the resulting mixture was extracted with DCM. The organic layer was separated, dried over MgSO₄, filtered and concentrated in vacuo. The residue thus obtained was purified by flash column chromatography (silica gel, EtOAc in heptane 0/100 to 100/0) and the desired fractions were concentrated in vacuo to yield Intermediate 6 (2.13 g; 94% yield) as orange oil.

Preparation of Intermediate 7

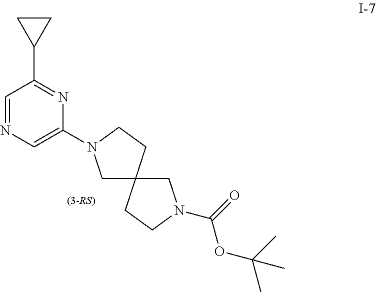 [SMILES: CC(C)(C)OC(=O)N1CCC2(CCN(c3cncc(C4CC4)n3)C2)C1]

Intermediate 6 (100 mg, 0.29 mmol) was added at rt to a deoxygenated a mixture of potassium cyclopropyltrifluoroborate (CAS: 1065010-87-8; 66 mg, 0.44 mmol), Pd(OAc)₂ (CAS 3375-31-3; 2.67 mg, 0.012 mmol), butyldi-1-adamantylphosphine (CAS 321921-71-5; 6.35 mg, 0.018 mmol), cesium carbonate (289 mg, 0.88 mmol) in toluene (2 mL) and water (0.38 mL). The mixture was heated at 100° C. for 16 h in a sealed tube. Water and DCM were added and the organic layer was separated, dried over Na₂SO₄, filtered and evaporated under vacuum. The residue thus obtained was purified by flash column chromatography (silica; MeOH in DCM, 0/100 to 5/95) and the desired fractions were concentrated in vacuo affording Intermediate 7 (81.8 mg, 80% yield).

Preparation of Intermediate 8

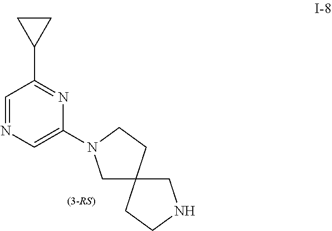 [SMILES: c1ncc(N2CCC3(CCNC3)C2)nc1C1CC1]

Trifluoroacetic acid (0.181 mL, 2.36 mmol) was added to a solution of Intermediate 7 (81.8 mg, 0.24 mmol) in DCM (1.06 mL) at rt under N₂ atmosphere. The mixture was stirred at rt for 14 h. The volatiles were evaporated under vacuo to yield Intermediate 8 (100 mg, 96% yield, trifluoroacetate salt).

Preparation of Intermediate 9

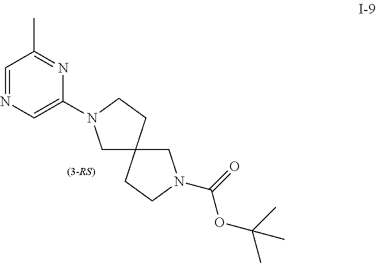 [SMILES: Cc1cncc(N2CCC3(CCN(C(=O)OC(C)(C)C)C3)C2)n1]

A mixture of Intermediate 6 (89.6 mg, 0.26 mmol), methylboronic acid (CAS: 13061-96-6; 19 mg, 0.32 mmol), Pd(PPh₃)₄ (CAS 14221-01-3, 31 mg, 0.026 mmol), and sodium carbonate (0.5 mL, aq. sat. soltn.) in 1,4-dioxane (7.75 mL) was heated at 150° C. for 15 min in a sealed tube under microwave irradiation. Water and DCM were added and the organic layer was separated, dried over MgSO₄, filtered and evaporated under vacuum. The residue thus obtained was purified by flash column chromatography (silica; EtOAc in DCM, 0/100 to 100/0) and the desired fractions were concentrated in vacuo affording Intermediate 9 (53 mg, 63% yield) as yellow oil.

Preparation of Intermediate 10

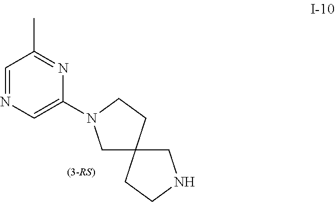 [SMILES: Cc1cncc(N2CCC3(CCNC3)C2)n1]

Trifluoroacetic acid (0.127 mL, 1.66 mmol) was added to a solution of Intermediate 9 (53 mg, 0.17 mmol) in DCM (0.5 mL) at rt and under N₂ atmosphere. The mixture was stirred at rt for 4 h. The volatiles were evaporated under vacuum affording a residue that was taken up in MeOH and passed through an isolute SCX-2 cartridge. The product was eluted with a 7N solution of NH₃ in MeOH. The volatiles were evaporated in vacuo affording Intermediate 10 (32 mg, 88% yield) as a pale yellow oil.

Preparation of Intermediate 11

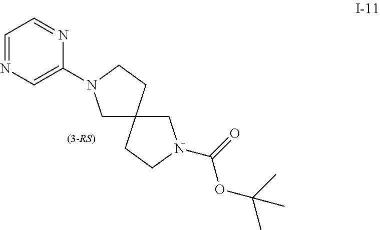 [SMILES: CC(C)(C)OC(=O)N1CCC2(CCN(c3cnccn3)C2)C1]

A solution of Intermediate 6 (122 mg, 0.36 mmol) in ethanol (7.2 mL) was hydrogenated in a H-Cube reactor (1 mL/min, 35 mm Pd/C cartridge, full H₂ mode, 50° C., 1 cycle). The solvent was evaporated under vacuum. The residue thus obtained was taken up in water and DCM. The organic layer was separated, dried over MgSO₄, filtered and evaporated under vacuum affording Intermediate 11 (71 mg, 65% yield) as a pale yellow oil.

Preparation of Intermediate 12

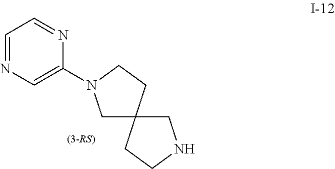 [SMILES: c1cnc(N2CCC3(CCNC3)C2)cn1]

Trifluoroacetic acid (0.176 mL, 2.3 mmol) was added to a solution of Intermediate 11 (70 mg, 0.23 mmol) in DCM (1 mL) at rt and under N₂ atmosphere. The mixture was stirred at rt for 16 h. The volatiles were evaporated under vacuum affording a residue that was taken up in MeOH and passed through an isolute SCX-2 cartridge. The product was eluted with a 7N solution of NH₃ in MeOH. The volatiles were evaporated in vacuo affording Intermediate 12 (37 mg, 79% yield) as a colorless oil.

Preparation of Intermediate 13

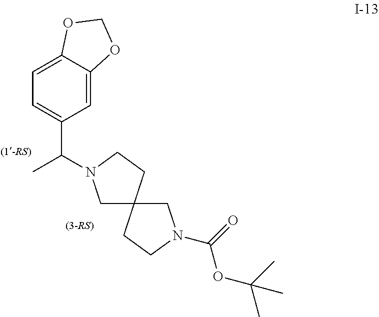 [SMILES: CC(c1ccc2c(c1)OCO2)N1CCC2(CCN(C(=O)OC(C)(C)C)C2)C1]

Sodium cyanoborohydride (417 mg, 6.63 mmol) was added to a stirred mixture of 2-Boc-2,7-diazaspiro[4.4]nonane (CAS: 236406-49-8; 1 g, 4.42 mmol), 3′,4′-(methylenedioxy)acetophenone (CAS 3162-29-6; 0.73 g, 4.42 mmol), Titanium(IV) isopropoxide (2.62 mL, 8.84 mmol) and triethylamine (1.23 mL, 8.84 mmol) in anhydrous MeOH (10.7 mL) under N₂ atmosphere. The suspension was stirred at 80° C. for 4 days.

Then water was added and the volatiles were evaporated under vacuum. Water was added and the mixture was extracted with a 1:2 mixture of 10% NH₃ in MeOH in DCM/DCM three times. The combined organic extracts were dried over MgSO₄, filtered and concentrated in vacuo. The residue thus obtained was purified by flash column chromatography (silica gel, MeOH in DCM 0/100 to 10/90) and the desired fractions were concentrated in vacuo to yield Intermediate 13 (1.2 g; 62% yield, 85% pure) as an amber oil.

Preparation of Intermediate 14

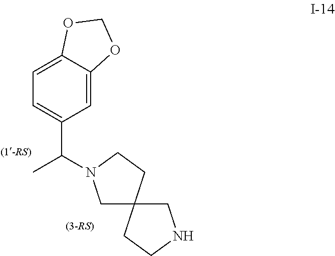 [SMILES: CC(c1ccc2c(c1)OCO2)N1CCC2(CCNC2)C1]

HCl (3 mL, 6M solution in isopropanol) was added to a solution of Intermediate 13 (0.6 g, 1.6 mmol) in DCM (12.3 mL) at rt. The mixture was stirred at rt for 16 h. The volatiles were evaporated under vacuo to yield crude Intermediate 14 (630 mg, HCl salt).

Preparation of Intermediate 15

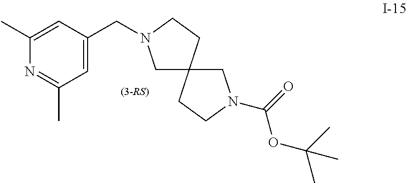 [SMILES: Cc1cc(CN2CCC3(CCN(C(=O)OC(C)(C)C)C3)C2)cc(C)n1]

Diisopropylethylamine (0.95 mL, 5.52 mmol) was added to a stirred suspension of 2-Boc-2,7-diazaspiro[4.4]nonane (CAS: 236406-49-8; 0.25 g, 1.1 mmol) in DCM (5.9 mL) at rt.

The mixture was stirred for 5 min and then 2,6-dimethylisonicotinaldehyde (CAS 18206-06-9; 179 mg, 1.3 mmol) and sodium triacetoxyborohydride (0.35 g, 1.66 mmol) were added. The mixture was stirred at rt for 16 hours. Then NaHCO₃ (aq. sat. soltn.) was added. The organic layer was separated, dried over MgSO₄, filtered and concentrated in vacuo. The residue thus obtained was purified by flash column chromatography (silica gel; MeOH in DCM 0/100 to 10/90) and the desired fractions were concentrated in vacuo to yield Intermediate 15 (0.25 g; 65% yield) as colorless syrup.

Preparation of Intermediate 16

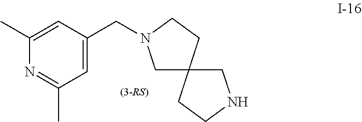 [SMILES: Cc1cc(CN2CCC3(CCNC3)C2)cc(C)n1]

HCl (1.8 mL, 4M solution in 1,4-dioxane) was added to a solution of Intermediate 15 (249 mg, 0.27 mmol) in 1,4-dioxane (3.8 mL) at rt. The mixture was stirred at rt for 16 h. The volatiles were evaporated under vacuo to yield Intermediate 16 (229 mg, quantitative, bis-HCl salt) as brownish solid.

PREPARATION OF INTERMEDIATES 17, 19, 21, 23, 26, 28, 30, 32, 34, 37, 39, 41, 43, 45, 47, 49, 51, 53, 55, 57, 59, 61, 63, 65, 67, 69, 71, 73, 75, 77, 79, 81, 83, 85, 87 and 89.

The intermediates in Table 1 were prepared following a procedure like the one described for the preparation of intermediate 3, starting from the corresponding Boc-protected amine intermediate using hydrochloric acid or trifluoroacetic acid under standard reaction conditions known to the person skilled in the art.

TABLE 1

| | | ACID/ |
|---|---|---|
| INTERMEDIATE AMINE | BOC-PROTECTED INTERMEDIATE AMINE | SOLVENT |
| 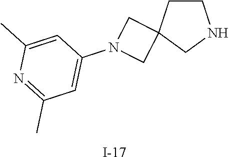 | 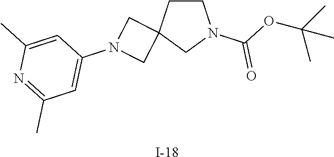 | HCl/MeOH |
| 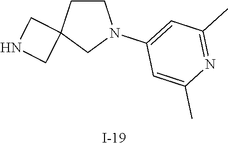 | 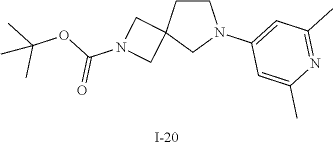 | HCl/MeOH |
| 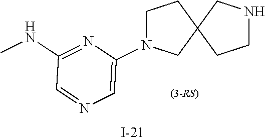 | 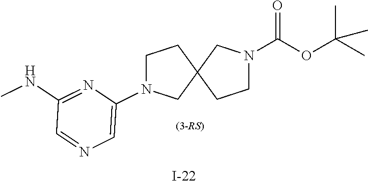 | TFA/DCM |
| 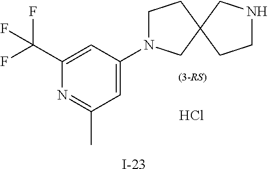 | 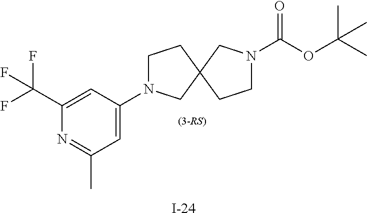 | HCl/MeOH |
| 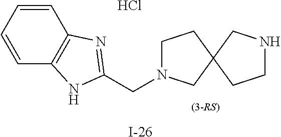 | 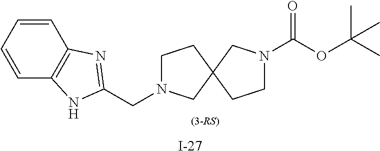 | HCl/ 1,4-dioxane |
| 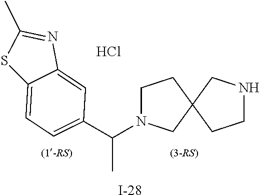 | 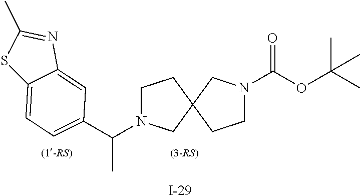 | HCl/ 1,4-dioxane |
| 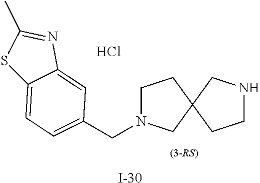 | 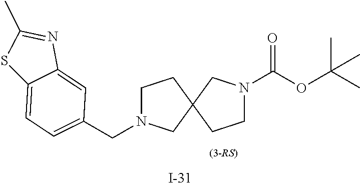 | HCl/ 1,4-dioxane |
| 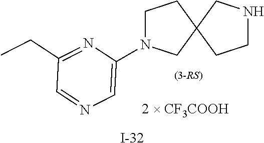 | 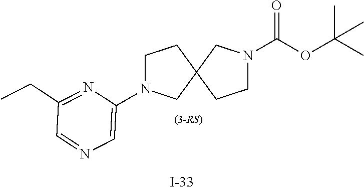 | TFA/DCM |
| 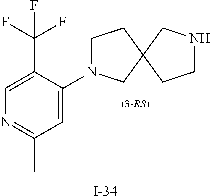 | 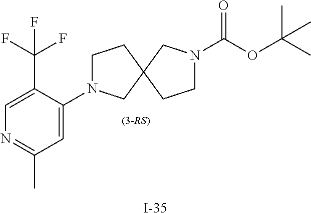 | HCl-IPA/ MeOH |
| 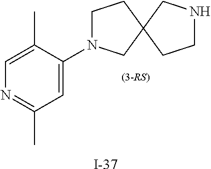 | 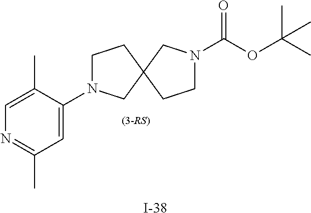 | TFA + HCl/ 1,4-dioxane |
| 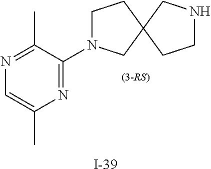 | 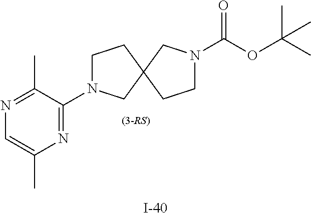 | HCl/ 1,4-dioxane |
| 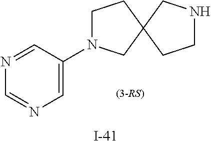 | 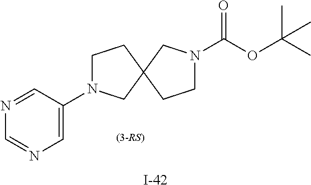 | TFA/DCM |
| 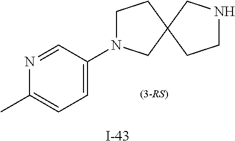 | 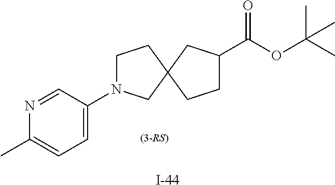 | TFA/DCM |
| 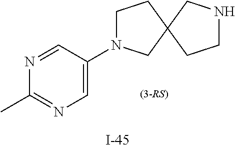 | 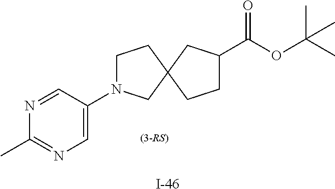 | TFA/DCM |
| 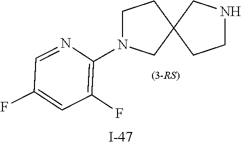 | 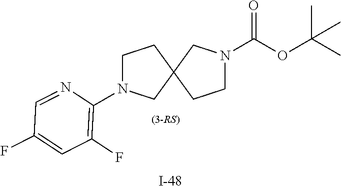 | TFA/DCM |
| 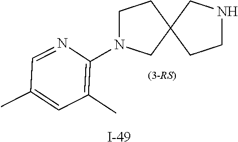 | 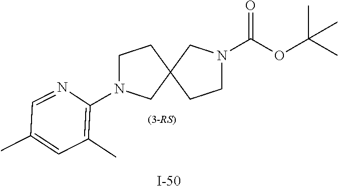 | TFA/DCM |
| 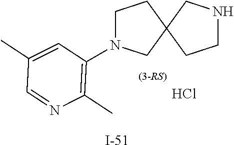 | 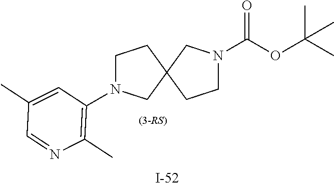 | HCl/EtOAc |
| 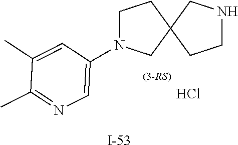 | 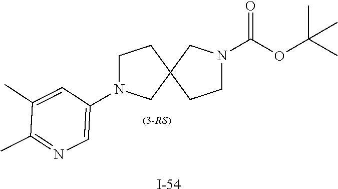 | HCl/EtOAc |
| 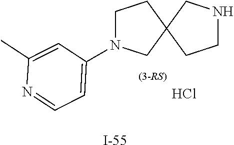 | 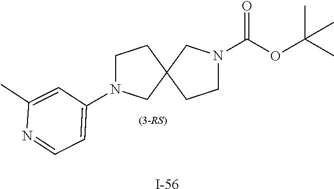 | TFA/DCM |
| 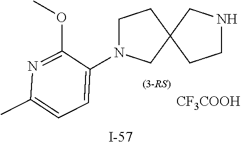 | 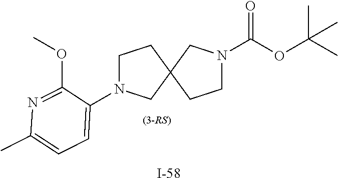 | TFA/— |
| 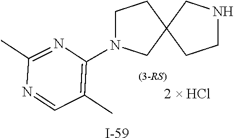 | 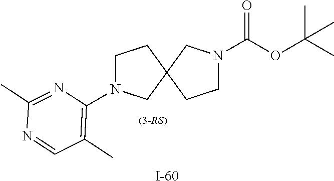 | HCl/ 1,4-dioxane |
| 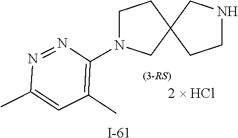 | 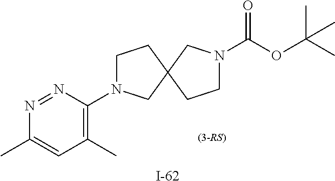 | HCl/ 1,4-dioxane |
| 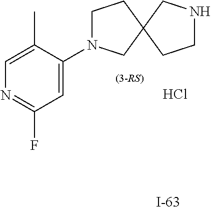 | 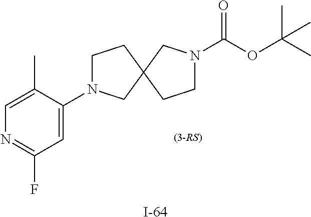 | HCl/EtOAc |
| 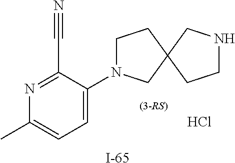 | 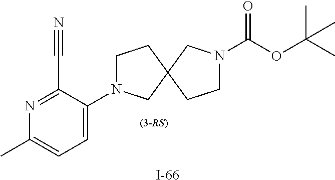 | HCl/EtOAc |
| 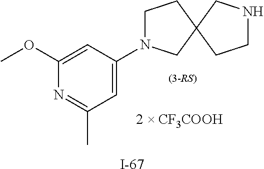 | 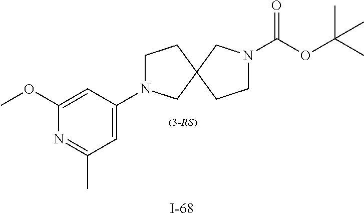 | TFA/DCM |
| 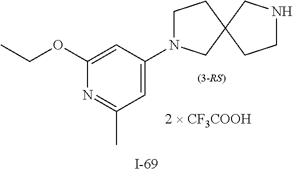 | 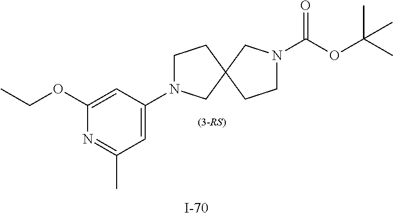 | TFA/DCM |
| 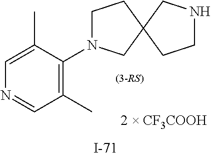 | 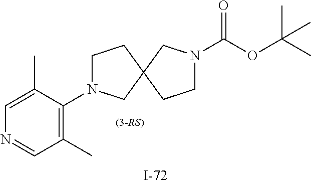 | TFA/DCM |
| 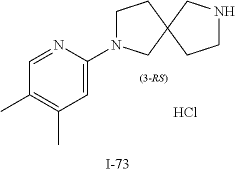 | 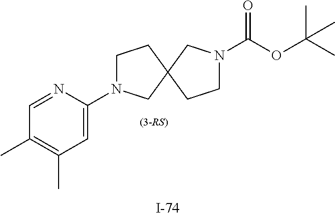 | HCl/ 1,4-dioxane |
| 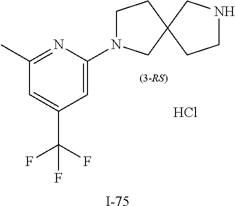 | 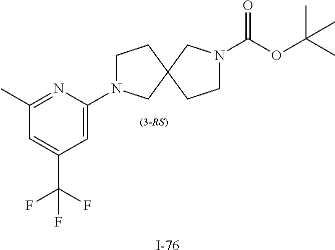 | HCl/ 1,4-dioxane |
| 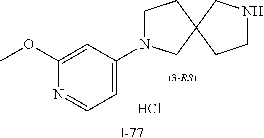 | 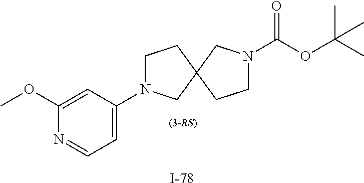 | HCl/ 1,4-dioxane |
| 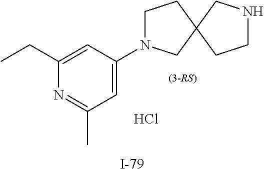 | 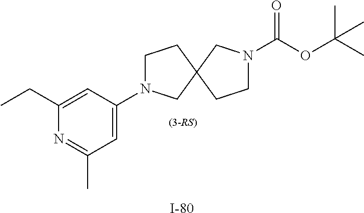 | HCl/ 1,4-dioxane |
| 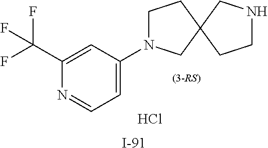 | 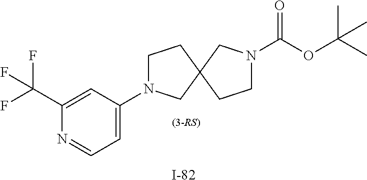 | HCl/ 1,4-dioxane |
| 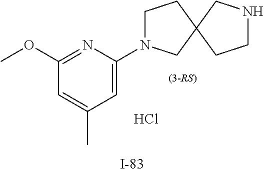 | 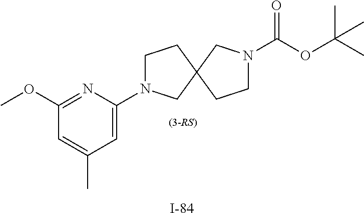 | HCl/ 1,4-dioxane |
| 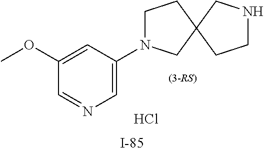 | 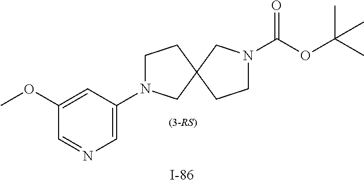 | HCl/ 1,4-dioxane |
| 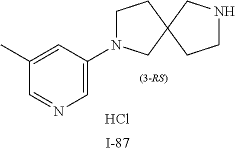 | 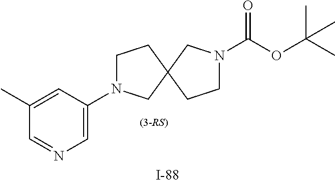 | HCl/ 1,4-dioxane |
| 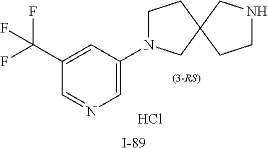 | 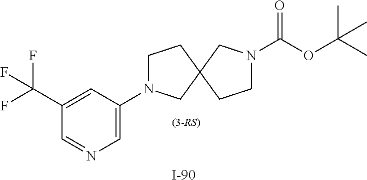 | HCl/ 1,4-dioxane |

PREPARATION OF INTERMEDIATES 18, 20 and 38

The compounds in Table 2 were prepared following a reaction procedure like the one described for the preparation of intermediate 2 starting from the corresponding spirodiamine intermediates and halo-substituted heteroaromatic intermediates under standard reaction conditions known to the person skilled in the art.

TABLE 2

| | | HALO-SUBSTITUTED |
|---|---|---|
| | SPIRODIAMINE | HETEROAROMATIC |
| INTERMEDIATE | INTERMEDIATE | INTERMEDIATES |
| 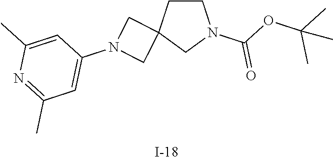 | CAS: 885270-86-0 | CAS: 3512-75-2 |
| 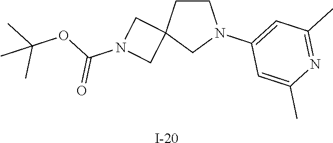 | CAS: 885270-84-8 | CAS: 3512-75-2 |
| 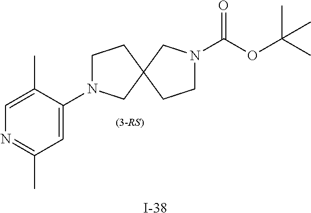 | CAS: 236406-49-8 | CAS: 22282-80-0 |

Preparation of Intermediate 22

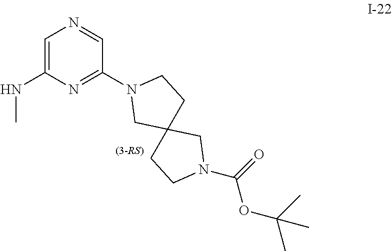 [SMILES: CNc1cncc(N2CCC3(CCN(C(=O)OC(C)(C)C)C3)C2)n1]

A mixture of tert-butyl 2,7-diazaspiro[4,4]nonane-2-carboxylate (CAS: 236406-49-8; 250 mg, 1.01 mmo), 6-chloro-N-methylpyrazin-2-amine (317 mg, 2.2 mmol), sodium tert-butoxide (318 mg, 3.3 mmol), 2-dicyclohexylphosphino-2′,6′-diisopropoxy-1,1′-biphenyl (CAS: 787618-22-8; 51.5 mg, 0.11 mmol), Pd(dba)₃ (CAS: 51364-51-3; 50.6 mg, 0.055 mmol) in toluene (7.5 mL) under N₂ atmosphere in a sealed tube was stirred at 100° C. for 16 h. The reaction mixture was filtered through diatomaceous earth, the filtrate was evaporated and the residue was purified by flash chromatography (silica; MeOH in DCM 0/100 to 5/95). The desired fractions were collected and evaporated to give intermediate 33 (169.6 mg, 46% yield) as a brown sticky oil.

Preparation of Intermediate 24

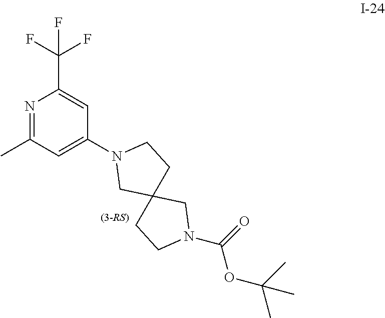 [SMILES: Cc1cc(N2CCC3(CCN(C(=O)OC(C)(C)C)C3)C2)cc(C(F)(F)F)n1]

Trimethylboroxine (0.197 mL, 1.4 mmol) was added to a stirred suspension of intermediate 25 (283 mg, 0.7 mmol), XPHOS Pd G3 (CAS: 1445085-55-1; 59 mg, 0.069 mmol) and cesium carbonate (454 mg, 1.4 mmol) in 1,4-dioxane (4.76 mL) in a sealed tube under N₂ atmosphere. The mixture was stirred at 120° C. for 10 min. under microwave irradiation. The mixture was diluted with EtOAc and washed with water. The organic layer was separated and washed with brine, dried (Na₂SO₄), filtered and concentrated in vacuo.

The residue was purified by flash column chromatography (SiO₂, EtOAc in heptane from 0/100 to 100/0). The desired fractions were collected and concentrated in vacuo to yield intermediate 24 (253 mg, 94% yield) as a colourless oil.

Preparation of Intermediate 25

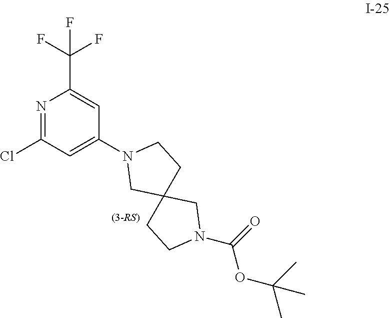 [SMILES: CC(C)(C)OC(=O)N1CCC2(CCN(c3cc(Cl)nc(C(F)(F)F)c3)C2)C1]

1,1′-Bis(diphenylphosphino)ferrocene-palladium(II)dichloride dichloromethane adduct (CAS: 95464-05-4; 53.7 mg, 0.065 mmol) was added to a stirred suspension of tert-butyl 2,7-diazaspiro[4.4]nonane-2-carboxylate (CAS: 236406-49-8; 294 mg, 1.3 mmol), 2-chloro-4-iodo-6-(trifluoromethyl)pyridine (400 mg, 1.3 mmol) and cesium carbonate (848 mg, 2.6 mmol) in toluene (4 mL) in a sealed tube under N₂ atmosphere. The mixture was stirred at 100° C. for 16 h. Then the mixture was diluted with EtOAc and washed with water. The organic layer was separated, dried (Na₂SO₄), filtered and concentrated in vacuo.

The residue was purified by flash column chromatography (SiO₂, EtOAc in heptane from 0/100 to 50/50). The desired fractions were collected and concentrated in vacuo to yield intermediate 25 (283 mg, 53% yield) as a pale yellow solid.

Preparation of Intermediate 27

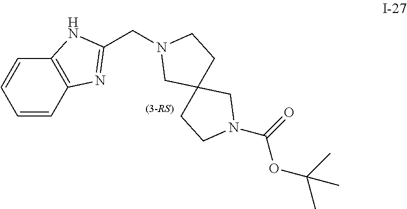 [SMILES: CC(C)(C)OC(=O)N1CCC2(CCN(Cc3nc4ccccc4[nH]3)C2)C1]

Sodium acetate (72 mg, 0.88 mmol) was added to a mixture of tert-butyl 2,7-diazaspiro[4.4]nonane-2-carboxylate (CAS: 236406-49-8; 80 mg, 0.3 mmol; HCl salt), 1H-benzimidazole-2-carboxaldehyde (59 mg, 0.36 mmol) in MeOH (10 mL) sodium acetate (72 mg, 0.88 mmol) at 0° C. After, the reaction was stirred for 30 min at rt, followed by the mixture reaction was cooled to 0° C., and acetic acid (18.2 mg, 0.3 mmol) and sodium cyanoborohydride (22 mg, 0.35 mmol) were added. The mixture was stirred rt overnight. Then additional acetic acid (2eq), 1H-benzimidazole-2-carboxaldehyde (leq) and sodium cyanoborohydride (1.5eq) were added at 0° C. and the mixture was stirred at rt overnight.

Water was added and the mixture was extracted with EtOAc (3×20 mL). Then, the organic phase was separated, dried over MgSO₄, filtered and concentrated in vacuo. The crude material was purified by flash chromatography (silica, gradient from MeOH/DCM(9:1) to DCM 0/100 to 100/0). The desired fractions were collected and concentrated in vacuo to yield intermediate 27 (60 mg, 55% yield) as a yellow oil.

Preparation of Intermediate 29

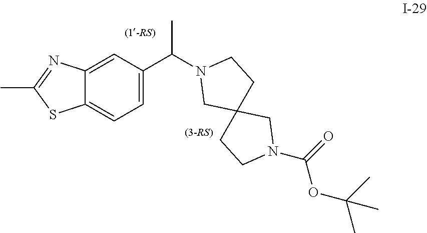 [SMILES: Cc1nc2cc(C(C)N3CCC4(CCN(C(=O)OC(C)(C)C)C4)C3)ccc2s1]

To a solution of tert-butyl 2,7-diazaspiro[4.4]nonane-2-carboxylate (CAS: 236406-49-8; 166.8 mg, 0.73 mmol) in anhydrous DCM (2.7 mL), 2-methyl-benzothiazole-5-carbaldehyde (196 mg, 1.1 mmol) and titanium(IV) isopropoxide (0.32 mL, 1.1 mmol) were added and the reaction mixture was stirred at rt for 18 h. Additional titanium(IV) isopropoxide (1.5eq) was added and the mixture was stirred at rt overnight. Then, the reaction was cooled to 0° C. and methyl magnesium bromide (2.63 mL, 3.69 mmol; 1.4 M in THF) was added dropwise followed by anhydrous THF (2.28 mL) and the reaction mixture was stirred at 0° C. for 5 min and at rt for 1.5 h. The mixture was diluted with NH₄Cl sat, filtered over diatomaceous earth and the mixture was extracted with EtOAc (3×10 mL). The organic layer was dried over MgSO₄ and filtered. The solvent was concentrated in vacuo. The crude material was purified by flash chromatography (silica, gradient from DCM/MeOH 9/19 to DCM 0/100 to 40/60). The desired fractions were collected and concentrated in vacuo to yield intermediate 29 (76 mg, 26% yield) as a yellow solid.

Preparation of Intermediate 31

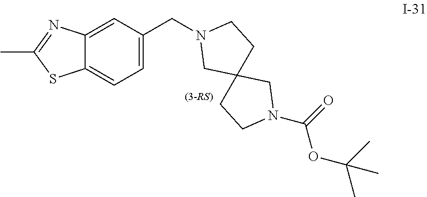 [SMILES: Cc1nc2cc(CN3CCC4(CCN(C(=O)OC(C)(C)C)C4)C3)ccc2s1]

Acetic acid (0.051 mL, 0.88 mmol) was added to a mixture of tert-butyl 2,7-diazaspiro[4.4]nonane-2-carboxylate (CAS: 236406-49-8; 100 mg, 0.44 mmol) and 2-methyl-benzothiazole-5-carbaldehyde (78 mg, 0.44 mmol) in MeOH (15 mL) at 0° C. After, the reaction was stirred for 30 min at 0° C. and then sodium cyanoborohydride (32 mg, 0.51 mmol) was added. The mixture was stirred rt overnight. NaHCO₃ (aq. sat. sltn.) was added and the mixture was extracted with EtOAc. Then, the organic phase was separated, dried over MgSO₄, filtered and concentrated in vacuo. The crude material was purified by flash chromatography (silica, gradient from DCM to DCM/MeOH 9:1). The desired fractions were collected and concentrated in vacuo to yield intermediate 31 (131 mg, 76% yield) as a colorless oil.

Preparation of Intermediate 33

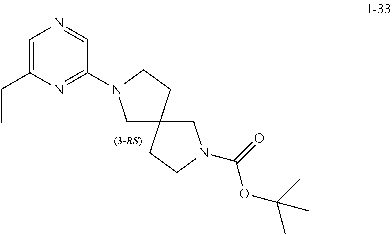 [SMILES: CCc1cncc(N2CCC3(CCN(C(=O)OC(C)(C)C)C3)C2)n1]

A mixture of tert-butyl 2,7-diazaspiro[4,4]nonane-2-carboxylate (CAS: 236406-49-8; 100 mg, 0.44 mmo), 2-chloro-6-ethylpyrazine (127 mg, 0.89 mmol), sodium tert-butoxide (127 mg, 1.32 mmol), 2-dicyclohexylphosphino-2′,6′-diisopropoxy-1,1′-biphenyl (CAS: 787618-22-8; 20.6 mg, 0.044 mmol), Pd(dba)₃ (CAS: 51364-51-3; 20.23 mg, 0.022 mmol) in toluene (3 mL) under N₂ atmosphere in a sealed tube was stirred at 100° C. for 16 h. The reaction mixture was filtered through diatomaceous earth, the filtrate was evaporated and the residue was purified by flash chromatography (silica; MeOH in DCM 0/100 to 5/95). The desired fractions were collected and evaporated to give intermediate 33 (90 mg, 61% yield) as a brown oil.

Preparation of Intermediate 35

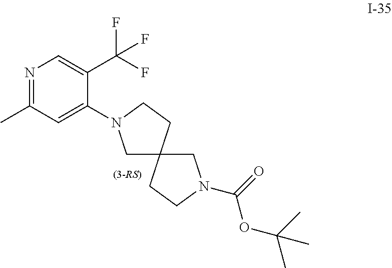 [SMILES: Cc1cc(N2CCC3(CCN(C(=O)OC(C)(C)C)C3)C2)c(C(F)(F)F)cn1]

1,1′-Bis(diphenylphosphino)ferrocene-palladium(II)dichloride dichoromethane adduct (CAS: 95464-05-4; 29 mg, 0.035 mmol) was added to a stirred suspension of tert-butyl 2,7-diazaspiro[4.4]nonane-2-carboxylate (CAS: 236406-49-8; 189 mg, 0.83 mmol), 4-iodo-2-methyl-5-(trifluoromethyl)pyridine (200 mg, 0.69 mmol) and cesium carbonate (454 mg, 1.39 mmol) in toluene (2.2 mL) in a sealed tube under N₂ atmosphere. The mixture was stirred at 100° C. for 16 h. Then the mixture was cooled to rt and extracted twice with EtOAc and washed with water. The organic layer was separated, dried (Na₂SO₄), filtered and concentrated in vacuo. The residue was purified by flash column chromatography (SiO₂, EtOAc in heptane from 20/80 to 100/0). The desired fractions were collected and concentrated in vacuo to yield intermediate 35 (175 mg, 65% yield) as a yellow oil.

Preparation of Intermediate 36

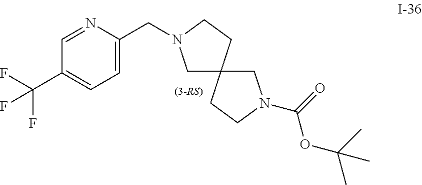 [SMILES: CC(C)(C)OC(=O)N1CCC2(CCN(Cc3ccc(C(F)(F)F)cn3)C2)C1]

Potassium carbonate (53 mg, 0.38 mmol) was added to a stirred solution of 2-(chloromethyl)-5-(trifluoromethyl)pyridine (50 mg, 0.2 mmol) and tert-butyl 2,7-diazaspiro[4.4]nonane-2-carboxylate (CAS: 236406-49-8; 87 mg, 0.38 mmol) in DMF (0.6 mL). The mixture was stirred at rt for 16 h. Then the mixture was diluted with EtOAc and washed with water. The organic layer was dried (Na₂SO₄), filtered and concentrated in vacuo. The residue was purified by flash column chromatography (SiO₂, EtOAc in heptane from 0/100 to 100/0). The desired fractions were collected and concentrated in vacuo to yield intermediate 36 (35 mg, 44% yield) as a colorless oil.

Preparation of Intermediate 40

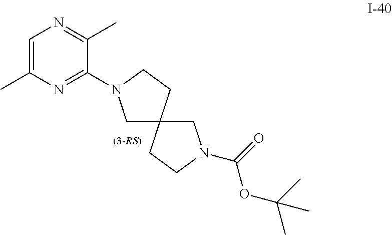 [SMILES: Cc1cnc(C)c(N2CCC3(CCN(C(=O)OC(C)(C)C)C3)C2)n1]

A solution of tert-butyl 2,7-diazaspiro[4.4]nonane-2-carboxylate (CAS: 236406-49-8; 201 mg, 0.88 mmol) in THF (3.9 mL) was added to a stirred mixture of 3-chloro-2,5-dimethylpyrazine (0.2 mL, 1.66 mmol), RUPHOS Pd G3 (CAS: 1445085-77-7; 86.7 mg, 0.1 mmol), 2-dicyclohexylphosphino-2′,6′-diisopropoxybiphenyl (37.4 mg, 0.08 mmol) and sodium tert-butoxide (130 mg, 1.36 mmol) in a sealed tube and under N₂ atmosphere. The mixture was stirred at 90° C. for 63 h. The mixture was treated with water and extracted with DCM. The organic layer was separated, dried (MgSO₄), filtered and the solvents evaporated in vacuo. The crude product was purified by flash column chromatography (SiO₂, EtOAc in heptane 0/100 to 100/0). The desired fractions were collected and concentrated in vacuo to yield intermediate 40 (225 mg, 77% yield) as yellow oil.

Preparation of Intermediate 42

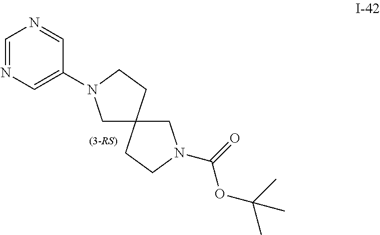 [SMILES: CC(C)(C)OC(=O)N1CCC2(CCN(c3cncnc3)C2)C1]

Pd₂(dba)₃ (37.6 mg, 0.039 mmol) and BINAP (CAS: 98327-87-8; 38.3 mg, 0.06 mmol) were added to a stirred mixture of tert-butyl 2,7-diazaspiro[4.4]nonane-2-carboxylate (CAS: 236406-49-8; 170 mg, 0.75 mmol), 5-bromopyrimidine (137 mg, 0.86 mmol) and cesium carbonate (411 mg, 1.26 mmol) in toluene at rt while a N₂ stream was bubbled though the mixture. Then the reaction mixture was stirred at 90° C. into a sealed tube and under N₂ atmosphere for 16 h. The mixture was cooled to rt and them it was filtered through diatomaceous earth and the diatomaceous earth pad was washed with EtOAc. The combined organic filtrates were evaporated in vacuo to yield crude intermediate 42 (262 mg, 66% yield, 58% pure) as an orange syrup. The compound was used in the next reaction step without further purification.

PREPARATION OF INTERMEDIATES 44, 46, 48, 50, 52, 54, 56, 58, 60, 62, 64, 66, 68, 70, 72, 74, 76, 78, 82, 84, 86, 88 and 90.

The compounds of Table 3 were prepared following a reaction procedure like the one described for the preparation of intermediate 42 starting from tert-butyl 2,7-diazaspiro[4.4]nonane-2-carboxylate (CAS: 236406-49-8) and the corresponding halo-substituted heteroaromatic intermediates under Buchwald coupling reaction conditions known to the person skilled in the art. Palladium catalyst, phosphine, base and solvent used are indicated in the table below.

TABLE 3

| | HALOSUBSTITUTED | | | |
|---|---|---|---|---|
| | HETEROAROMATIC | | | BASE/SOLVENT |
| INTERMEDIATE | INTERMEDIATE (CAS) | CATALYST | PHOSPHINE | TEMPERATURE |
| 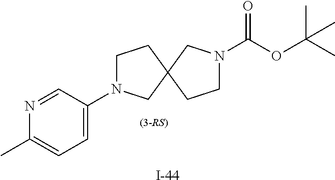 | 3430-13-5 | Pd₂(dba)₃ | BINAP | Cs₂CO₃/Toluene/90° C. |
| 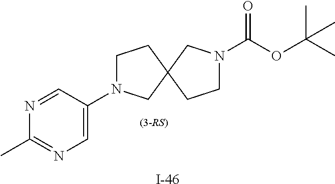 | 4595-59-9 | Pd₂(dba)₃ | BINAP | Cs₂CO₃/Toluene/90° C. |
| 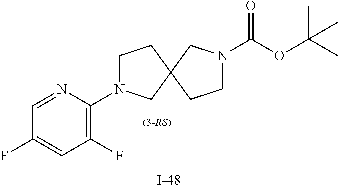 | 660425-16-1 | Pd₂(dba)₃ | BINAP | Cs₂CO₃/Toluene/90° C. |
| 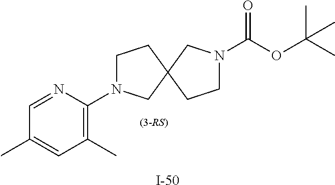 | 30838-93-8 | Pd₂(dba)₃ | BINAP | Cs₂CO₃/Toluene/90° C. |
| 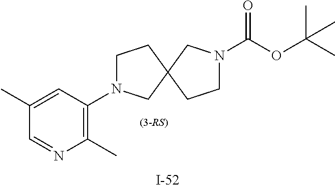 | 17117-19-0 | Pd₂(dba)₃ | BINAP | Cs₂CO₃/Toluene/90° C. |
| 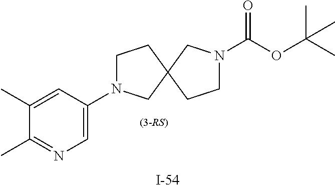 | 27063-90-7 | Pd₂(dba)₃ | BINAP | Cs₂CO₃/Toluene/90° C. |
| 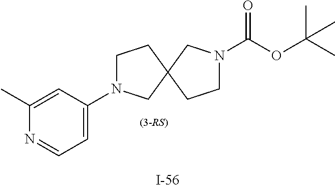 | 22282-99-1 | Pd₂(dba)₃ | BINAP | Cs₂CO₃/Toluene/90° C. |
| 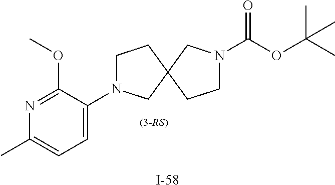 | 717843-47-5 | Pd₂(dba)₃ | BINAP | Cs₂CO₃/Toluene/90° C. |
| 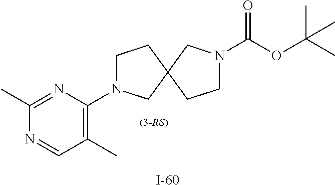 | 75715-74-2 | Pd₂(dba)₃ | BINAP | Cs₂CO₃/Toluene/90° C. |
| 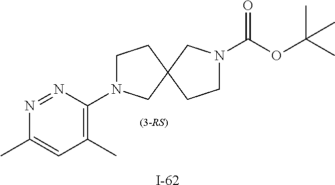 | 17258-26-3 | Pd₂(dba)₃ | BINAP | Cs₂CO₃/Toluene/90° C. |
| 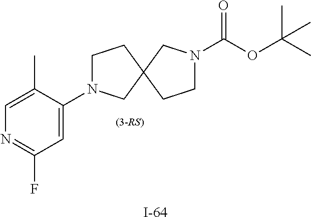 | 1227577-02-7 | Pd₂(dba)₃ | BINAP | Cs₂CO₃/Toluene/90° C. |
| 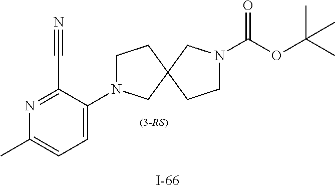 | 717843-48-6 | Pd₂(dba)₃ | CAS: 564483-18-7 | Cs₂CO₃/Toluene/90° C. |
| 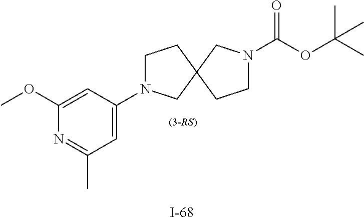 | 1083169-00-9 | Pd₂(dba)₃ | BINAP | Cs₂CO₃/Toluene/90° C. |
| 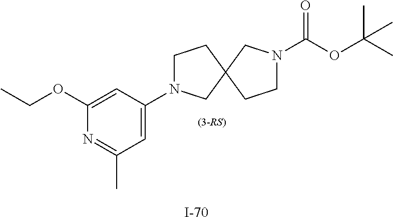 | 1300633-96-8 | Pd₂(dba)₃ | BINAP | Cs₂CO₃/Toluene/90° C. |
| 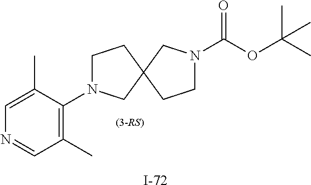 | 201286-65-9 | Pd₂(dba)₃ | BINAP | Cs₂CO₃/Toluene/90° C. |
| 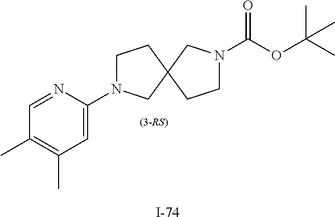 | 343268-69-9 | Pd₂(dba)₃ | CAS: 564483-18-7 | Cs₂CO₃/Toluene/90° C. |
| 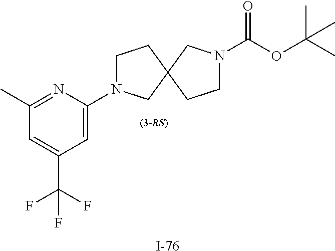 | 22123-14-4 | Pd₂(dba)₃ | CAS: 564483-18-7 | Cs₂CO₃/Toluene/90° C. |
| 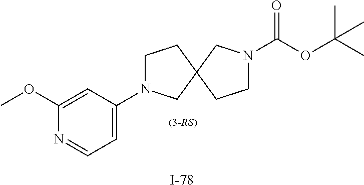 | 72141-44-7 | Pd₂(dba)₃ | CAS: 564483-18-7 | Cs₂CO₃/Toluene/90° C. |
| 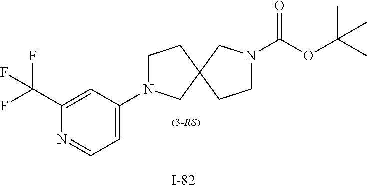 | 131748-14-6 | Pd₂(dba)₃ | CAS: 564483-18-7 | Cs₂CO₃/Toluene/90° C. |
| 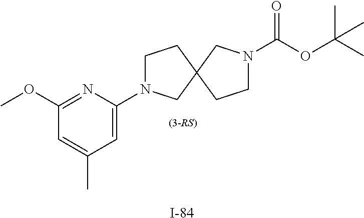 | 25297-52-3 | Pd₂(dba)₃ | CAS: 564483-18-7 | Cs₂CO₃/Toluene/90° C. |
| 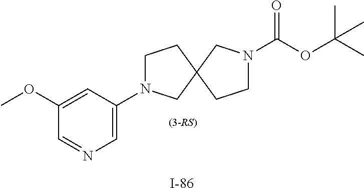 | 50720-12-2 | Pd₂(dba)₃ | CAS: 564483-18-7 | Cs₂CO₃/Toluene/90° C. |
| 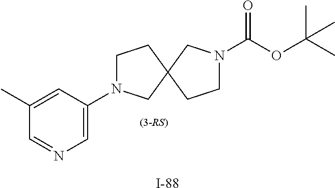 | 3430-16-8 | Pd₂(dba)₃ | CAS: 564483-18-7 | Cs₂CO₃/Toluene/90° C. |
| 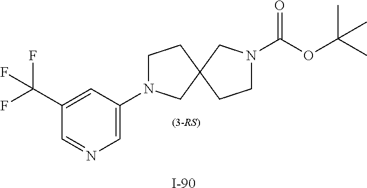 | 436799-33-6 | Pd₂(dba)₃ | CAS: 564483-18-7 | Cs₂CO₃/Toluene/90° C. |

Preparation of Intermediate 80

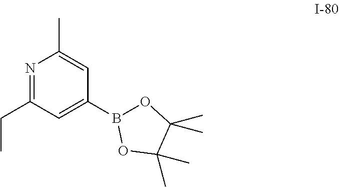 [SMILES: CCc1cc(B2OC(C)(C)C(C)(C)O2)cc(C)n1]

A mixture of 2-ethyl-6-methylpyridine (500 mg, 4.1 mmol), bis(pinacolato)diboron (1 g, 4.1 mmol) and 4,4′-di-tert-butyl-2,2′-bipyridine (22 mg, 0.082 mmol) in octane (20 mL) was stirred at room temperature for 15 min. Then 1,5-cyclooctadiene-iridium(I) chloride dimer (CAS: 12112-67-3; 27.7 mg, 0.041 mmol) was added and the mixture was stirred at 80° C. for 6 h. The reaction mixture was cooled to rt and diluted with DCM (50 mL). Water (15 mL) was added and the mixture was stirred for 15 min. The water phase was extracted with dichloromethane (6×50 mL*6). The combined organic phases were dried with anhydrous MgSO₄ and concentrated under reduced pressure to give intermediate 80 (800 mg, 96% yield) as a black oil.

Preparation of Intermediate 81

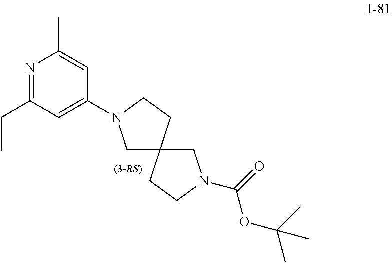 [SMILES: CCc1cc(N2CCC3(CCN(C(=O)OC(C)(C)C)C3)C2)cc(C)n1]

A mixture of intermediate 80 (500 mg, 2 mmol), tert-butyl 2,7-diazaspiro[4.4]nonane-2-carboxylate (CAS: 236406-49-8; 457 mg, 2 mmol), Cu(OAc)₂ (81 mg, 0.4 mmol) and pyridine (480 mg, 6 mmol) in DMF (10 mL) was stirred at 80° C. for overnight. The reaction mixture was filtered and the filtrate was concentrated under vacuum to give crude intermediate 81 that was purified with Prep.HPLC (Column: Xtimate C18 150*25 mm*5 um; condition: water(0.225% FA)-CAN; begin B: 18, end B: 48; Gradient Time(min): 7; 100% B Hold Time(min): 2; FlowRate(ml/min): 25. The pure fractions were collected and the solvent was evaporated under vacuum to give intermediate 81 (100 mg, 13% yield) as orange oil.

Preparation of Intermediate 92

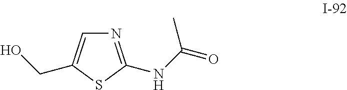

Lithium triethylborohydride (2.8 mL, 2.8 mmol; 1M solution in THF) was added to a solution of intermediate 1 (200 mg, 0.93 mmol) in THF (4.6 mL) cooled at −78° C. The mixture was allowed to warm to rt and then further stirred at rt for 16 h. Water and EtOAc were added and the organic phase was separated and discarded. The aqueous phase was evaporated to dryness and the resulting solid was washed with water, filtered, dried and purified by reverse phase HPLC (Stationary phase: C18 XBridge 30×100 mm 5 um), mobile phase: gradient from 90% 0.1% NH₄CO₃H/NH₄OH pH 9 solution in water, 10% CH₃CN to 0% 0.1% NH₄CO₃H/NH₄OH pH 9 solution in water, 100% CH₃CN). The desired fractions were concentrated in vacuo to yield intermediate 92 as a white solid (50 mg, 31% yield).

Preparation of Intermediate 93

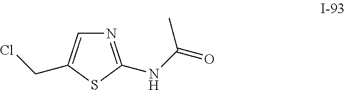

Sulfonyl chloride (0.042 mL, 0.51 mmol) was added to a solution of intermediate 92 (100 mg, 0.48 mmol) in DCM (3.05 mL) at 0° C. The mixture was allowed to warm to rt and then further stirred at rt for 1 h. The volatiles were evaporated in vacuo affording intermediate 93 as yellow solid (98 mg, 91% yield).

### B. PREPARATION OF FINAL COMPOUNDS

Preparation of Product 1

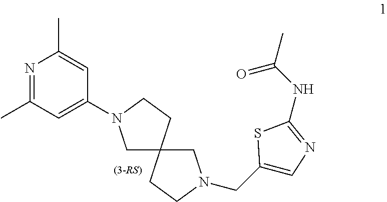 [SMILES: CC(=O)Nc1ncc(CN2CCC3(CCN(c4cc(C)nc(C)c4)C3)C2)s1]

Diisopropylethylamine (0.21 mL, 1.23 mmol) was added to a stirred suspension of Intermediate 3 (57 mg, 0.25 mmol) in DCM (1.31 mL) at rt and the mixture was stirred at rt for 5 min. Then, Intermediate 1 (50 mg, 0.3 mmol) and sodium triacetoxyborohydride (78.3 mg, 0.37 mmol) were added and the mixture was further stirred at rt for 16 h. The reaction mixture was quenched with NaHCO₃ (aq. sat. soltn.). The organic layer was separated, dried over MgSO₄, filtered and the filtrate was evaporated in vacuo. The residue thus obtained was purified by flash column chromatography (silica gel, MeOH in DCM, 0/100 to 25/75). The desired fractions were concentrated in vacuo to yield a residue that was triturated with diisopropylether to yield product 1 (38 mg, 40% yield) as a white solid.

Preparation of Product 2

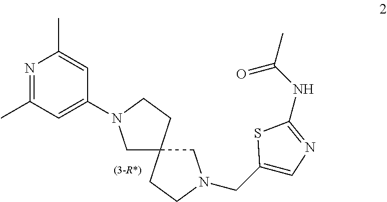

HCl (2 mL, 4M solution in 1,4-dioxane) was added to a stirred solution of Intermediate 2a (263 mg, 0.79 mmol) in 1,4-dioxane (5 mL) at rt. The solution was stirred at rt for 16 h. Then the solvent was evaporated affording a residue that was taken up in MeOH and passed through an isolute SCX-2 cartridge. The product was eluted with a 7N solution of NH₃ in MeOH. The volatiles were evaporated in vacuo. The residue thus obtained was dissolved in DCM (4 mL) and then Intermediate 1 (190 mg, 1.12 mmol) and sodium triacetoxyborohydride (261 mg, 1.23 mmol) were added under N₂ atmosphere and the mixture was further stirred at rt for 60 h. Then, NaHCO₃ (aq. sat. soltn.) and DCM were added to the mixture. The organic layer was separated, dried over MgSO₄, filtered and the filtrate was evaporated in vacuo. The residue thus obtained was purified by flash column chromatography (silica gel, 7N solution of NH₃ in MeOH in DCM, 0/100 to 10/90). The desired fractions were concentrated in vacuo to yield product 2 (23 mg, 7.5% yield) as pale yellow oil.

Preparation of Product 3

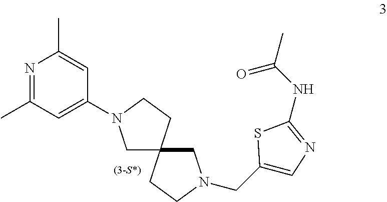

HCl (2.2 mL, 4M solution in 1,4-dioxane) was added to a stirred solution of Intermediate 2b (289 mg, 0.87 mmol) in 1,4-dioxane (5 mL) at rt. The solution was stirred for at rt for 16 h. Then the solvent was evaporated affording a residue that was taken up in MeOH and passed through an isolute SCX-2 cartridge. The product was eluted with a 7N solution of NH₃ in MeOH. The volatiles were evaporated in vacuo. The residue thus obtained was dissolved in DCM (4 mL) and then Intermediate 1 (176 mg, 1.03 mmol) and sodium triacetoxyborohydride (259 mg, 1.22 mmol) were added under N₂ atmosphere and the mixture was further stirred at rt for 60 h. Then, NaHCO₃ (aq. sat. soltn.) and DCM were added to the mixture. The organic layer was separated, dried over MgSO₄, filtered and the filtrate was evaporated in vacuo. The residue thus obtained was purified by flash column chromatography (silica gel, 7N solution of NH₃ in MeOH in DCM, 0/100 to 10/90). The desired fractions were concentrated in vacuo to yield product 3 (30 mg, 9% yield) as pale yellow oil.

Preparation of Product 4

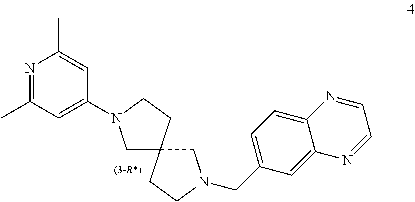

Acetic acid (0.03 mL, 0.52 mmol) was added to a stirred solution of Intermediate 3a (55 g, 0.24 mmol) and 6-quinoxalinecarboxaldehyde (CAS: 130345-50-5; 49 mg, 0.31 mmol) in MeOH (1 mL) at rt. The solution was stirred for at rt for 2.5 h. Then sodium cyanoborohydride (37 mg, 0.59 mmol) was added and the mixture was further stirred at rt for 60 h. Then, NaHCO₃ (aq. sat. soltn.) and DCM were added to the mixture. The organic layer was separated, dried over MgSO₄, filtered and the filtrate was evaporated in vacuo.

The crude product was purified by RP HPLC (Stationary phase: C18 XBridge 30×100 mm 5 μm), mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and extracted with EtOAc and DCM/2-PrOH (9/1). The desired fractions were collected and concentrated in vacuo. The crude product was purified by ion exchange chromatography (ISOLUTE SCX-2, MeOH and then 7N solution of NH₃ in MeOH). The desired fractions were collected and concentrated in vacuo to yield to yield product 4 (20.3 mg, 23% yield) as yellow oil.

Preparation of Product 5

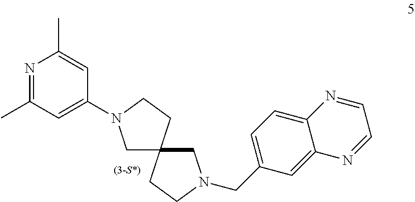

Acetic acid (0.03 mL, 0.52 mmol) was added to a stirred solution of Intermediate 3b (54 mg, 0.23 mmol) and 6-quinoxalinecarboxaldehyde (CAS: 130345-50-5; 53 mg, 0.33 mmol) in MeOH (1 mL) at rt. The solution was stirred for at rt for 2.5 h. Then sodium cyanoborohydride (43 mg, 0.68 mmol) was added and the mixture was further stirred at rt for 60 h. Then, NaHCO₃ (aq. sat. soltn.) and DCM were added to the mixture. The organic layer was separated, dried over MgSO₄, filtered and the filtrate was evaporated in vacuo. The crude product was purified by RP HPLC (Stationary phase: C18 XBridge 30×100 mm 5 μm), mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and extracted with EtOAc and DCM/2-PrOH (9/1). The desired fractions were collected and concentrated in vacuo. The crude product was purified by ion exchange chromatography (ISOLUTE SCX-2, MeOH and then 7N solution of NH₃ in MeOH). The desired fractions were collected and concentrated in vacuo to yield to yield product 5 (16.5 mg, 19% yield) as yellow oil.

Preparation of Product 6

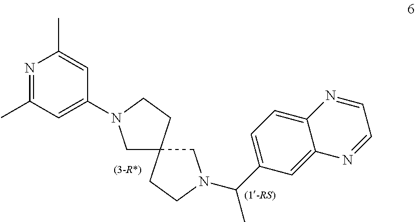

Titanium tetraisopropoxide (0.1 mL, 0.34 mmol) was added to a stirred suspension of Intermediate 3a (71 mg, 0.31 mmol) and 1-(quinoxalin-6-yl)ethanone (CAS: 83570-42-7; 63 mg, 0.37 mmol) in THF (1.5 mL) at rt and under N₂ atmosphere. The mixture was stirred into a sealed tube at 80° C. for 16 h. Then sodium cyanoborohydride (30 mg, 0.48 mmol) was added and the mixture was further stirred at 80° C. for 16 h. Then, NaHCO₃ (aq. sat. soltn.) and DCM were added to the mixture. The solvent was evaporated in vacuo and the crude product was purified by flash column chromatography (silica gel, 7N solution of NH₃ in MeOH in DCM, from 0/100 to 10/90) and then by RP HPLC (Stationary phase: C18 XBridge 30×100 mm 5 μm; mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo to yield product 6 (15 mg, 13% yield) as yellow oil.

Preparation of Product 7

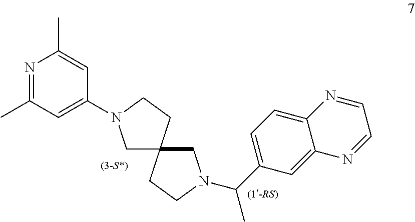

Titanium tetraisopropoxide (0.08 mL, 0.27 mmol) was added to a stirred suspension of Intermediate 3b (57 mg, 0.25 mmol) and 1-(quinoxalin-6-yl)ethanone (CAS: 83570-42-7; 50 mg, 0.29 mmol) in THF (1.5 mL) at rt and under N₂ atmosphere. The mixture was stirred into a sealed tube at 80° C. for 16 h. Then sodium cyanoborohydride (28 mg, 0.45 mmol) was added and the mixture was further stirred at 80° C. for 16 h. Then, NaHCO₃ (aq. sat. soltn.) and DCM were added to the mixture. The solvent was evaporated in vacuo and the crude product was purified by flash column chromatography (silica gel, 7N solution of NH₃ in MeOH in DCM, from 0/100 to 10/90) and then by RP HPLC (Stationary phase: C18 XBridge 30×100 mm 5 μm; mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo to yield product 7 (10 mg, 10% yield) as yellow oil.

Preparation of Product 8

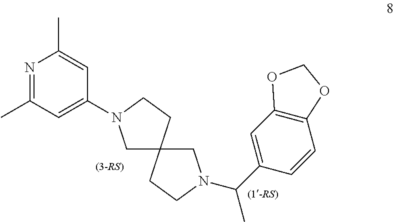 [SMILES: Cc1cc(N2CCC3(CCN(C(C)c4ccc5c(c4)OCO5)C3)C2)cc(C)n1]

Sodium cyanoborohydride (20 mg, 0.32 mmol) was added to a stirred mixture of Intermediate 3 (50 mg, 0.22 mmol), 3′,4′-(methylenedioxy)acetophenone (CAS 3162-29-6; 35 mg, 0.22 mmol), triethylamine (0.06 mL, 0.423 mmol) and titanium tetraisopropoxide (0.128 mL, 0.43 mmol) in anhydrous MeOH (0.53 mL) at rt. Then the mixture was stirred at 80° C. for 72 h. The reaction mixture was quenched with water and the volatiles were evaporated under vacuum. Water was then added and the mixture was extracted three times with EtOAc. The combined organic extracts were washed with brine, dried over Na₂SO₄, filtered and the filtrate was evaporated in vacuo. The crude product was purified by RP HPLC (stationary phase: C18 XBridge 30×100 mm 5 μm; mobile phase: gradient from 67% 10 mM NH₄CO₃H pH 9 solution in water, 33% CH₃CN to 50% 10 mM NH₄CO₃H pH 9 solution in water, 50% CH₃CN). The desired fractions were concentrated in vacuo to yield product 8 (22 mg, 27% yield) as an oil.

Preparation of Product 9

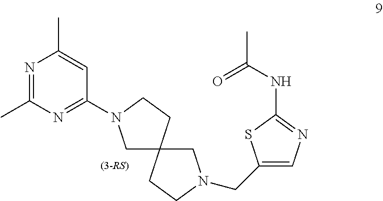 [SMILES: CC(=O)Nc1ncc(CN2CCC3(CCN(c4cc(C)nc(C)n4)C3)C2)s1]

Diisopropylethylamine (0.16 mL, 0.93 mmol) was added to a stirred suspension of Intermediate 5 (50 mg, 0.19 mmol) in DCM (1 mL) at rt and the mixture was further stirred at rt for 5 min. Then, Intermediate 1 (38 mg, 0.22 mmol) and sodium triacetoxyborohydride (59 mg, 0.28 mmol) were added and the mixture was further stirred at rt for 16 h. The reaction mixture was quenched with NaHCO₃ (aq. sat. soltn.). The organic layer was separated, dried over MgSO₄, filtered and the filtrate was evaporated in vacuo. The residue thus obtained was purified by flash column chromatography (silica gel, MeOH in DCM, 0/100 to 25/75). The desired fractions were concentrated in vacuo to yield a residue that was triturated with diisopropylether to yield product 9 (36 mg, 50% yield) as a white solid.

Preparation of Product 10

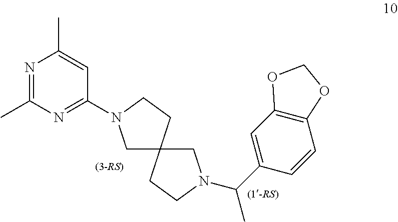 [SMILES: Cc1cc(N2CCC3(CCN(C(C)c4ccc5c(c4)OCO5)C3)C2)nc(C)n1]

Sodium cyanoborohydride (18 mg, 0.28 mmol) was added to a stirred mixture of Intermediate 5 (50 mg, 0.19 mmol), 3′,4′-(methylenedioxy)acetophenone (CAS 3162-29-6; 30 mg, 0.19 mmol), triethylamine (0.05 mL, 0.372 mmol) and titanium tetraisopropoxide (0.11 mL, 0.372 mmol) in anhydrous MeOH (0.45 mL) at rt. Then the mixture was stirred at 80° C. for 72 h. The reaction mixture was quenched with water and the volatiles were evaporated under vacuum. Water was then added and the mixture was extracted three times with EtOAc. The combined organic extracts were washed with brine, dried over Na₂SO₄, filtered and the filtrate was evaporated in vacuo. The crude product was purified by RP HPLC (stationary phase: C18 XBridge 30×100 mm 5 μm; mobile phase: gradient from 67% 10 mM NH₄CO₃H pH 9 solution in water, 33% CH₃CN to 50% 10 mM NH₄CO₃H pH 9 solution in water, 50% CH₃CN). The desired fractions were concentrated in vacuo to yield product 10 (20 mg, 28% yield) as colorless oil.

Preparation of Product 11

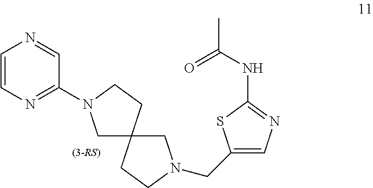 [SMILES: CC(=O)Nc1ncc(CN2CCC3(CCN(c4cnccn4)C3)C2)s1]

Intermediate 1 (28 mg, 0.16 mmol) and sodium triacetoxyborohydride (56 mg, 0.26 mmol) were added to a stirred solution of Intermediate 12 (30 mg, 0.15 mmol) in DCM (1 mL) at rt. The mixture was further stirred at rt for 60 h. The reaction mixture was quenched with NaHCO₃ (aq. sat. soltn.) and extracted with DCM. The organic layer was separated, dried over MgSO₄, filtered and the filtrate was evaporated in vacuo. The crude product was purified by RP HPLC (stationary phase: C18 XBridge 30×100 mm 5 μm; mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and extracted with EtOAc. The organic layer was separated, dried (MgSO₄), filtered and the solvents evaporated in vacuo to yield product 11 (7 mg, 13% yield) as a white solid.

Preparation of Product 12

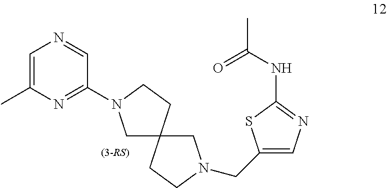 [SMILES: CC(=O)Nc1ncc(CN2CCC3(CCN(c4cncc(C)n4)C3)C2)s1]

Sodium triacetoxyborohydride (70 mg, 0.31 mmol) was added to a stirred solution of Intermediate 10 (32 mg, 0.15 mmol) and Intermediate 1 (29 mg, 0.17 mmol) in DCM (1 mL) at rt and under N₂ atmosphere. The mixture was further stirred at rt for 17 h. The reaction mixture was quenched with NaHCO₃ (aq. sat. soltn.) and extracted with DCM. The organic layer was separated, dried over MgSO₄, filtered and the filtrate was evaporated in vacuo. The crude product was purified by RP HPLC (stationary phase: C18 XBridge 30×100 mm 5 μm; mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and extracted with EtOAc. The organic layer was separated, dried (MgSO₄), filtered and the solvents evaporated in vacuo to yield product 12 (29 mg, 53% yield) as pale yellow oil.

Preparation of Product 13

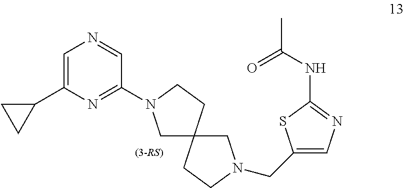 [SMILES: CC(=O)Nc1ncc(CN2CCC3(CCN(c4cncc(C5CC5)n4)C3)C2)s1]

Sodium triacetoxyborohydride (34 mg, 0.16 mmol) was added to a stirred solution of Intermediate 8 (20 mg, 0.08 mmol) and Intermediate 1 (19.8 mg, 0.12 mmol) in DCM (1 mL) at rt and under N₂ atmosphere. The mixture was further stirred at rt for 17 h. The reaction mixture was quenched with NaHCO₃ (aq. sat. soltn.) and extracted with DCM. The organic layer was separated, dried over MgSO₄, filtered and the filtrate was evaporated in vacuo. The crude product was purified by RP HPLC (stationary phase: C18 XBridge 30×100 mm 5 μm; mobile phase: gradient from 74% 10 mM NH₄CO₃H pH 9 solution in water, 26% CH₃CN to 58% 10 mM NH₄CO₃H pH 9 solution in water, 42% CH₃CN). The desired fractions were collected and extracted with EtOAc. The organic layer was separated, dried (MgSO₄), filtered and the solvents evaporated in vacuo to yield product 13 (15.5 mg, 53% yield) as pale yellow oil.

Preparation of Product 14

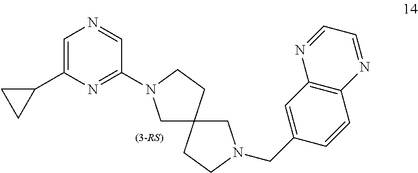 [SMILES: c1cnc2cc(CN3CCC4(CCN(c5cncc(C6CC6)n5)C4)C3)ccc2n1]

Diisopropylethylamine (0.17 mL, 0.98 mmol) was added to a stirred suspension of Intermediate 8 (70 mg, 0.19 mmol) in DCM (1 mL) at rt and the mixture was stirred at rt for 5 min. Then, 6-quinoxalinecarboxaldehyde (CAS: 130345-50-5; 38 mg, 0.22 mmol) and sodium triacetoxyborohydride (62 mg, 0.29 mmol) were added and the mixture was further stirred at rt for 16 h. The reaction mixture was quenched with NaHCO₃ (aq. sat. soltn.). The organic layer was separated, dried over MgSO₄, filtered and the filtrate was evaporated in vacuo. The residue thus obtained was purified by flash column chromatography (silica gel, MeOH in DCM, 0/100 to 10/90). The desired fractions were concentrated in vacuo to yield product 14 (43 mg, 57% yield) as a white solid.

Preparation of Product 15

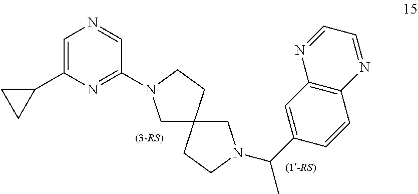 [SMILES: CC(c1ccc2nccnc2c1)N1CCC2(CCN(c3cncc(C4CC4)n3)C2)C1]

Titanium tetraisopropoxide (0.09 mL, 0.31 mmol) and 6-quinoxalinecarboxaldehyde (CAS: 130345-50-5; 49 mg, 0.31 mmol) were added to a stirred mixture of Intermediate 8 (50 mg, 0.2 mmol) in DCM (0.63 mL) at rt. The mixture was stirred at rt for 18 h. Then, the reaction mixture was cooled to 0° C. and methylmagnesium bromide (0.73 mL, 1.02 mmol; 1.4 M solution in THF) was added followed by THF (0.6 mL). The mixture was stirred at 0° C. for 5 min and then at rt for 3 h. The reaction mixture was quenched with NH₄Cl (aq. sat. soltn.) and extracted with DCM. The organic layer was separated, dried over Na₂SO₄, filtered and the filtrate was evaporated in vacuo. The residue thus obtained was purified by flash column chromatography (silica gel, MeOH in DCM, 0/100 to 10/90). The desired fractions were concentrated in vacuo to yield product 15 (20 mg, 24% yield) as a brown sticky solid.

Preparation of Product 16

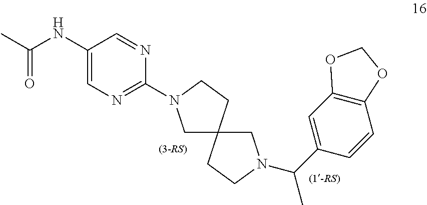 [SMILES: CC(=O)Nc1cnc(N2CCC3(CCN(C(C)c4ccc5c(c4)OCO5)C3)C2)nc1]

N-(2-Chloropyrimidin-5-yl)acetamide (CAS 1353776-97-2; 0.89 mg, 0.36 mmol) was added to a stirred solution of Intermediate 14 (130 mg, 0.42 mmol) and diisopropylethylamine (0.13 mL, 0.91 mmol) in isopropanol (1.7 mL) at rt. The mixture was stirred at 100° C. for 16 h and then the volatiles were evaporated in vacuo. The residue thus obtained was purified by flash column chromatography (silica gel, MeOH in DCM, 0/100 to 10/90). The desired fractions were concentrated in vacuo to yield a crude product that was further purified by RP HPLC (stationary phase: C18 XBridge 30×100 mm 5 μm; mobile phase: gradient from 90% 10 mM NH₄CO₃H pH 9 solution in water, 10% CH₃CN to 0% 10 mM NH₄CO₃H pH 9 solution in water, 100% CH₃CN). The desired fractions were concentrated in vacuo to yield product 16 (40 mg, 27% yield) as a solid.

Preparation of Product 17

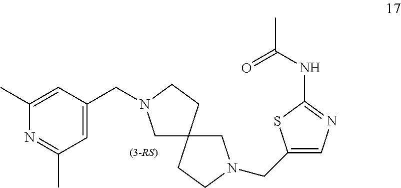 [SMILES: CC(=O)Nc1ncc(CN2CCC3(CCN(Cc4cc(C)nc(C)c4)C3)C2)s1]

Diisopropylethylamine (0.15 mL, 0.89 mmol) was added to a stirred suspension of Intermediate 16 (50 mg, 0.18 mmol) in DCM (1 mL) at rt and the mixture was stirred at rt for 5 min. Then, Intermediate 1 (36 mg, 0.21 mmol) and sodium triacetoxyborohydride (56 mg, 0.27 mmol) were added and the mixture was further stirred at rt for 16 h. The reaction mixture was quenched with NaHCO₃ (aq. sat. soltn.). The organic layer was separated, dried over MgSO₄, filtered and the filtrate was evaporated in vacuo. The residue thus obtained was purified by flash column chromatography (silica gel, MeOH in DCM, 0/100 to 25/75). The desired fractions were concentrated in vacuo to yield a crude product that was further purified by RP HPLC (stationary phase: C18 XBridge 30×100 mm 5 μm; mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were concentrated in vacuo to yield product 17 (19.5 mg, 27% yield) as a colorless oil.

Preparation of Product 18

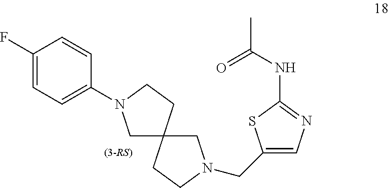 [SMILES: CC(=O)Nc1ncc(CN2CCC3(CCN(c4ccc(F)cc4)C3)C2)s1]

Intermediate 93 (228.5 mg, 0.74 mmol, 85% pure) was added to a solution of, 2-(4-fluorophenyl)-2,7-diazaspiro[4.4]nonane (CAS: 1368001-80-2, 135.3 mg, 0.61 mmol) and DIPEA (0.53 mL, 3.1 mmol) in 1,2-dichloroethane (3.4 mL) at 0° C. The mixture was stirred at rt for 30 min. Then the solvent was concentrated in vacuo. The residue was purified by reverse phase HPLC (stationary phase: C18 XBridge 30×100 mm 5 um), mobile phase: gradient from 67% 10 mM NH₄CO₃H pH 9 solution in water, 33% CH₃CN to 50% 10 mM NH₄CO₃H pH 9 solution in water, 50% CH₃CN), yielding product 18 (17 mg, 7.4% yield) as a pale solid.

PREPARATION OF PRODUCTS 19, 21, 22, 23, 25, 26, 28, 33, 34, 35 and 40.

The compounds of Table 4 were prepared following a reductive amination procedure like the one described for the preparation of product 1 starting from the corresponding amine and aldehyde intermediates using sodium triacetoxyborohydride in DCM.

TABLE 4

| | | INTER- | |
|---|---|---|---|
| | | MEDI- | |
| | | ATE | |
| | | ALDE- | |
| PRODUCT | INTERMEDIATE AMINE | HYDE | COMMENT |
| 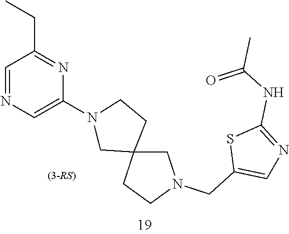 [SMILES: CCc1cncc(N2CCC3(CCN(Cc4cnc(NC(C)=O)s4)C3)C2)n1] | 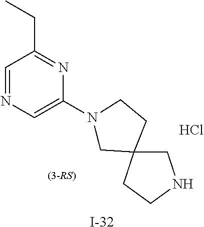 | I-1 | Base: DIPEA |
| 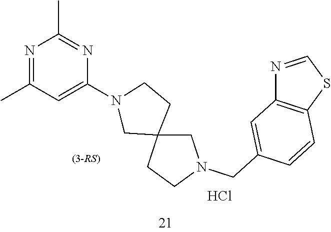 [SMILES: Cc1cc(N2CCC3(CCN(Cc4ccc5scnc5c4)C3)C2)nc(C)n1 ; Cl] | I-5 | CAS: 394233- 38-2 | Comment: HCl salt prepared |
| 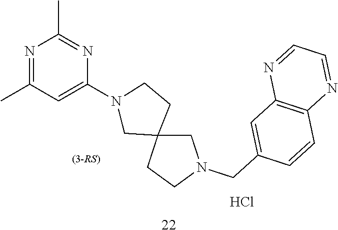 | I-5 | CAS: 130345- 50-5 | Comment: HCl salt prepared |
| 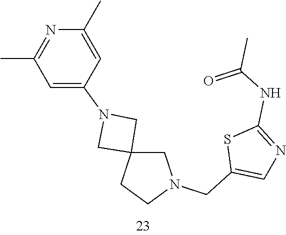 [SMILES: CC(=O)Nc1ncc(CN2CCC3(C2)CN(c2cc(C)nc(C)c2)C3)s1] | 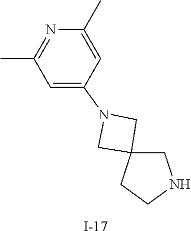 | I-1 | Co- solvent: MeOH |
| 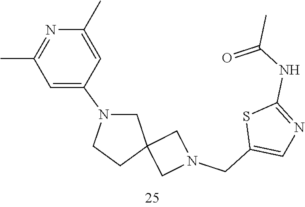 [SMILES: CC(=O)Nc1ncc(CN2CC3(CCN(c4cc(C)nc(C)c4)C3)C2)s1] | 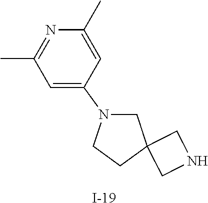 | I-1 | Co- solvent: MeOH |
| 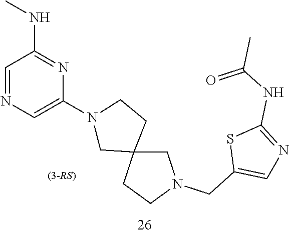 [SMILES: CNc1cncc(N2CCC3(CCN(Cc4cnc(NC(C)=O)s4)C3)C2)n1] | 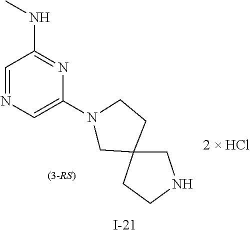 | I-1 | Base: DIPEA |
| 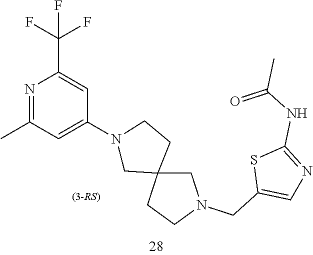 [SMILES: CC(=O)Nc1ncc(CN2CCC3(CCN(c4cc(C)nc(C(F)(F)F)c4)C3)C2)s1] | 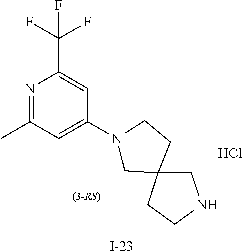 | I-1 | Co- solvent: MeOH; Base: DIPEA |
| 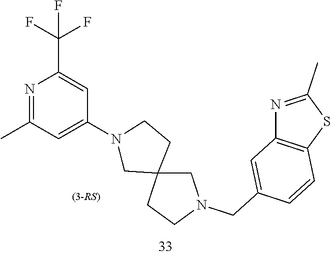 [SMILES: Cc1cc(N2CCC3(CCN(Cc4ccc5sc(C)nc5c4)C3)C2)cc(C(F)(F)F)n1] | 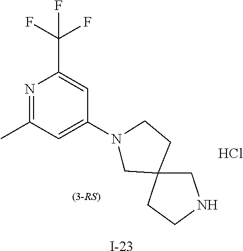 | CAS: 20061- 46-5 | Co- solvent: MeOH; Base: DIPEA |
| 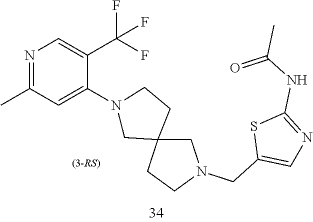 [SMILES: CC(=O)Nc1ncc(CN2CCC3(CCN(c4cc(C)ncc4C(F)(F)F)C3)C2)s1] | 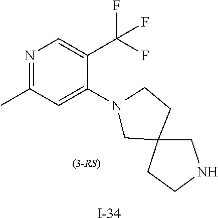 | I-1 | Co- solvent: MeOH |
| 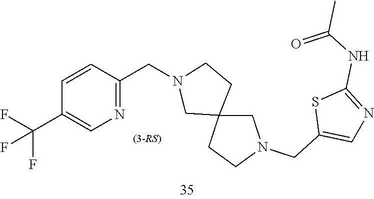 [SMILES: CC(=O)Nc1ncc(CN2CCC3(CCN(Cc4ccc(C(F)(F)F)cn4)C3)C2)s1] | 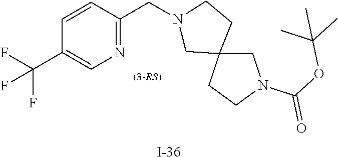 | I-1 | Co- solvent: MeOH; Base: triethyl- amine^{#} |
| 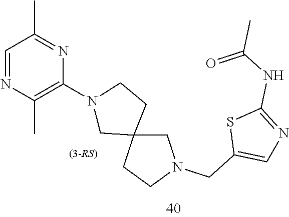 [SMILES: CC(=O)Nc1ncc(CN2CCC3(CCN(c4nc(C)cnc4C)C3)C2)s1] | 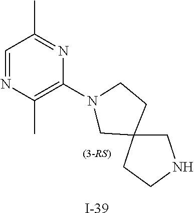 | I-1 | Base: DIPEA |

^{#}Before the reductive amination step the tert-butoxycarbonyl group was cleaved by treatment with HCl (6N in isopropanol)

PREPARATION OF PRODUCTS 39, 43-46, and 49-69.

The compounds in Table 5 were prepared following a reductive amination procedure like the one described for the preparation of product 5 starting from the corresponding amine and intermediate 1 using sodium cyanoborohydride, sodium acetate and acetic acid in MeOH.

TABLE 5

| PRODUCT | INTERMEDIATE AMINE |
|---|---|
| 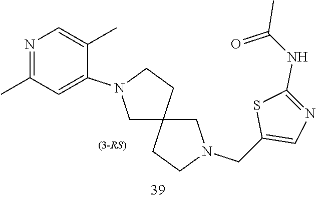 [SMILES: CC(=O)Nc1ncc(CN2CCC3(CCN(c4cc(C)ncc4C)C3)C2)s1] | 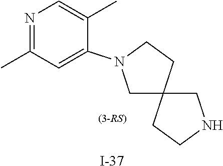 |
| 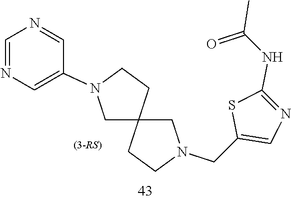 [SMILES: CC(=O)Nc1ncc(CN2CCC3(CCN(c4cncnc4)C3)C2)s1] | 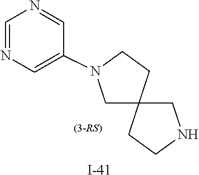 |
| 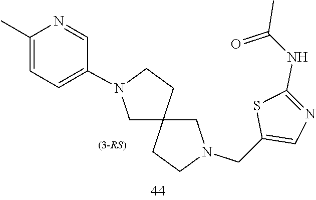 [SMILES: CC(=O)Nc1ncc(CN2CCC3(CCN(c4ccc(C)nc4)C3)C2)s1] | 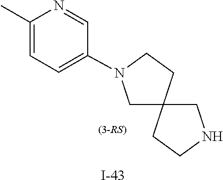 |
| 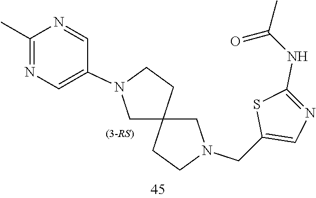 [SMILES: CC(=O)Nc1ncc(CN2CCC3(CCN(c4cnc(C)nc4)C3)C2)s1] | 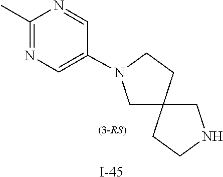 |
| 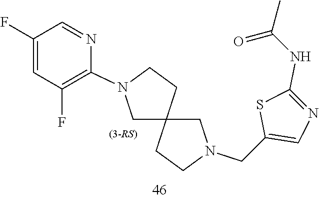 [SMILES: CC(=O)Nc1ncc(CN2CCC3(CCN(c4ncc(F)cc4F)C3)C2)s1] | 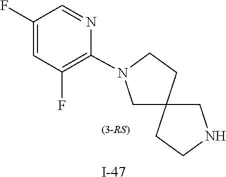 |
| 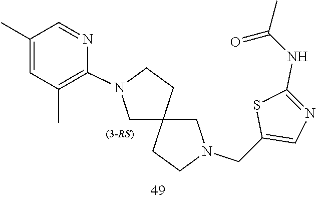 [SMILES: CC(=O)Nc1ncc(CN2CCC3(CCN(c4ncc(C)cc4C)C3)C2)s1] | 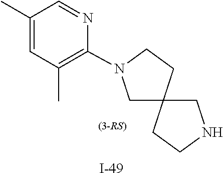 |
| 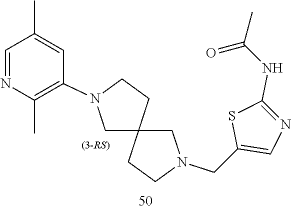 [SMILES: CC(=O)Nc1ncc(CN2CCC3(CCN(c4cc(C)cnc4C)C3)C2)s1] | 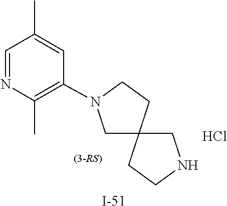 |
| 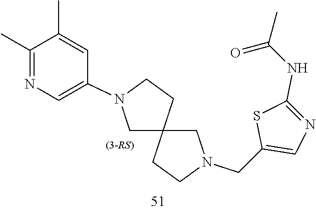 [SMILES: CC(=O)Nc1ncc(CN2CCC3(CCN(c4cnc(C)c(C)c4)C3)C2)s1] | 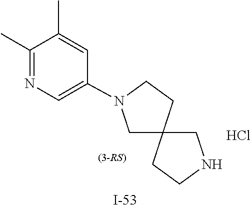 |
| 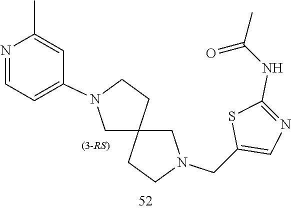 [SMILES: CC(=O)Nc1ncc(CN2CCC3(CCN(c4ccnc(C)c4)C3)C2)s1] | 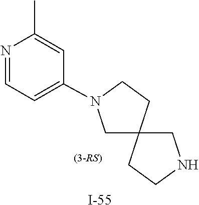 |
| 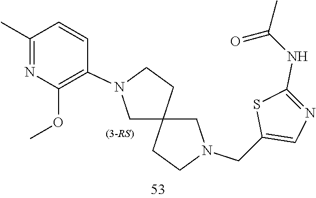 [SMILES: COc1nc(C)ccc1N1CCC2(CCN(Cc3cnc(NC(C)=O)s3)C2)C1] | 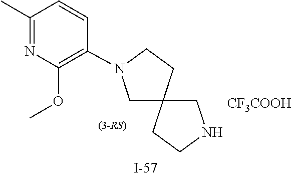 |
| 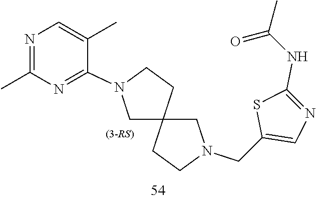 [SMILES: CC(=O)Nc1ncc(CN2CCC3(CCN(c4nc(C)ncc4C)C3)C2)s1] | 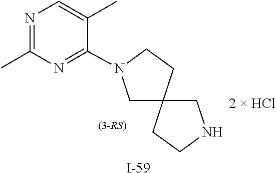 |
| 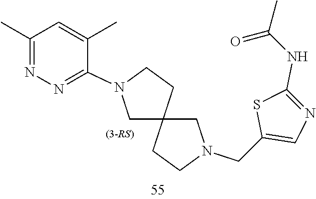 [SMILES: CC(=O)Nc1ncc(CN2CCC3(CCN(c4nnc(C)cc4C)C3)C2)s1] | 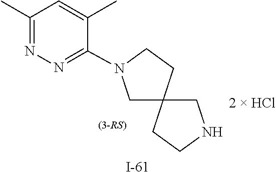 |
| 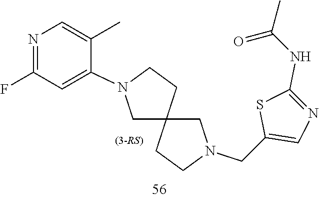 [SMILES: CC(=O)Nc1ncc(CN2CCC3(CCN(c4cc(F)ncc4C)C3)C2)s1] | 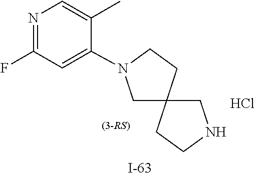 |
| 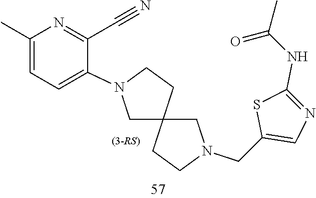 [SMILES: CC(=O)Nc1ncc(CN2CCC3(CCN(c4ccc(C)nc4C#N)C3)C2)s1] | 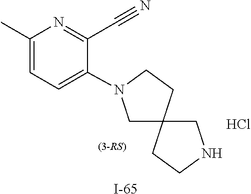 |
| 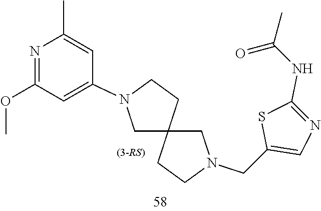 [SMILES: COc1cc(N2CCC3(CCN(Cc4cnc(NC(C)=O)s4)C3)C2)cc(C)n1] | 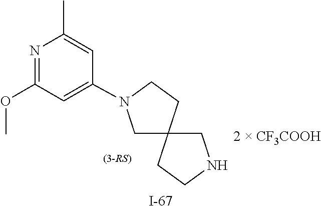 |
| 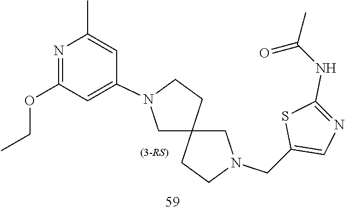 [SMILES: CCOc1cc(N2CCC3(CCN(Cc4cnc(NC(C)=O)s4)C3)C2)cc(C)n1] | 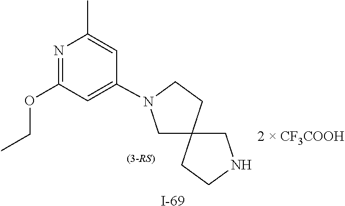 |
| 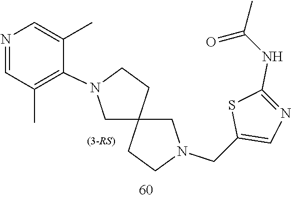 [SMILES: CC(=O)Nc1ncc(CN2CCC3(CCN(c4c(C)cncc4C)C3)C2)s1] | 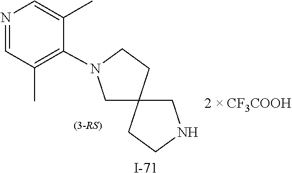 |
| 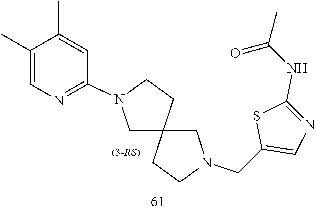 [SMILES: CC(=O)Nc1ncc(CN2CCC3(CCN(c4cc(C)c(C)cn4)C3)C2)s1] | 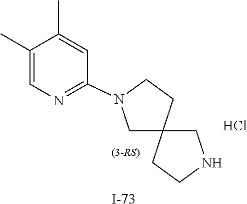 |
| 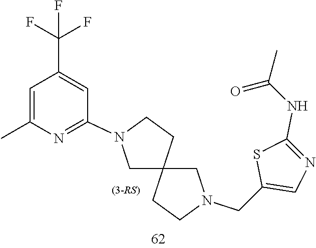 [SMILES: CC(=O)Nc1ncc(CN2CCC3(CCN(c4cc(C(F)(F)F)cc(C)n4)C3)C2)s1] | 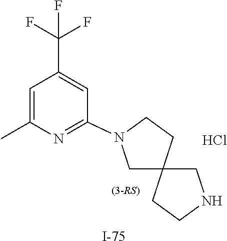 |
| 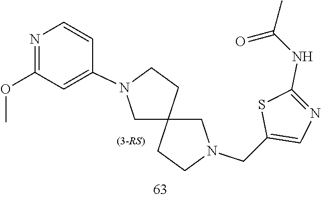 [SMILES: COc1cc(N2CCC3(CCN(Cc4cnc(NC(C)=O)s4)C3)C2)ccn1] | 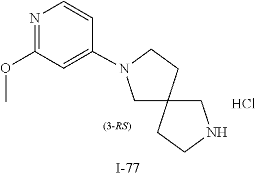 |
| 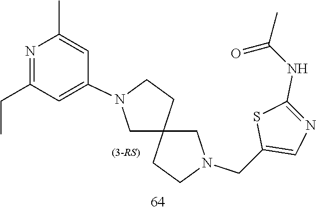 [SMILES: CCc1cc(N2CCC3(CCN(Cc4cnc(NC(C)=O)s4)C3)C2)cc(C)n1] | 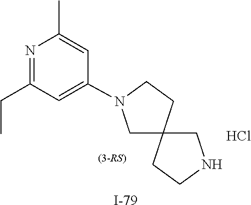 |
| 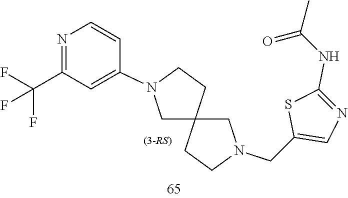 [SMILES: CC(=O)Nc1ncc(CN2CCC3(CCN(c4ccnc(C(F)(F)F)c4)C3)C2)s1] | 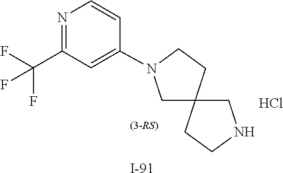 |
| 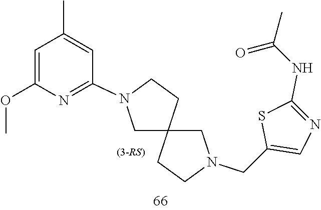 [SMILES: COc1cc(C)cc(N2CCC3(CCN(Cc4cnc(NC(C)=O)s4)C3)C2)n1] | 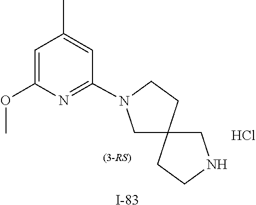 |
| 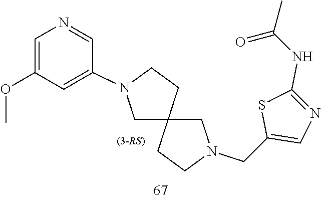 [SMILES: COc1cncc(N2CCC3(CCN(Cc4cnc(NC(C)=O)s4)C3)C2)c1] | 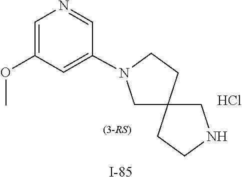 |
| 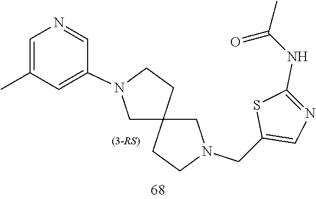 [SMILES: CC(=O)Nc1ncc(CN2CCC3(CCN(c4cncc(C)c4)C3)C2)s1] | 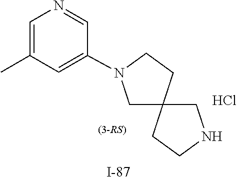 |
| 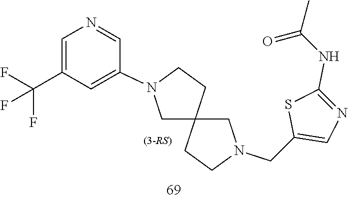 [SMILES: CC(=O)Nc1ncc(CN2CCC3(CCN(c4cncc(C(F)(F)F)c4)C3)C2)s1] | 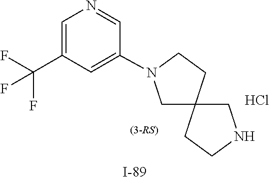 |

PREPARATION OF PRODUCTS 20, 24 and 27.

The compounds in Table 6 were prepared following a reductive amination procedure like the one described for the preparation of product 15 starting from intermediate 5 and the corresponding aldehyde intermediate using titanium tetraisopropoxyde and methyl magnesium bromide in MeOH/THF. After isolation of the corresponding compounds these were transformed into the HCl salts by treatment with HCl (4N in 1,4-dioxane).

TABLE 6

| PRODUCT | INTERMEDIATE ALDEHYDE (CAS) |
|---|---|
| 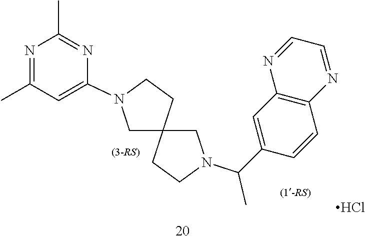 [SMILES: Cc1cc(N2CCC3(CCN(C(C)c4ccc5nccnc5c4)C3)C2)nc(C)n1 ; Cl] | 130345-50-5 |
| 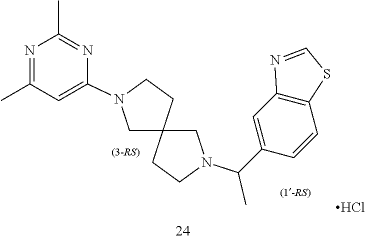 [SMILES: Cc1cc(N2CCC3(CCN(C(C)c4ccc5scnc5c4)C3)C2)nc(C)n1 ; Cl] | 394223-38-2 |
| 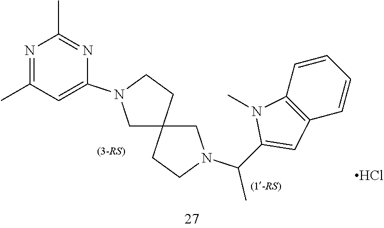 [SMILES: Cc1cc(N2CCC3(CCN(C(C)c4cc5ccccc5n4C)C3)C2)nc(C)n1 ; Cl] | 27421-51-8 |

Preparation of Products 29, and 36-38.

The compounds of Table 7 were prepared following a reductive amination procedure like the one described for the preparation of product 6 starting from intermediate 3b and the corresponding ketone intermediate using titanium tetraisopropoxyde and sodium cyanoborohydride in MeOH.

TABLE 7

| PRODUCT | INTERMEDIATE KETONE (CAS) |
|---|---|
| 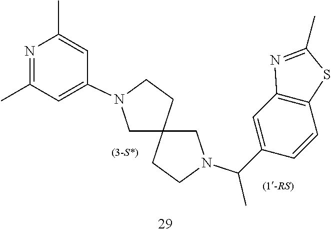 [SMILES: Cc1cc(N2CCC3(CCN(C(C)c4ccc5sc(C)nc5c4)C3)C2)cc(C)n1] | 20077-88-7 |
| 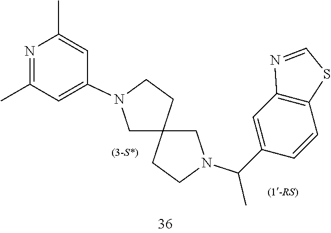 [SMILES: Cc1cc(N2CCC3(CCN(C(C)c4ccc5scnc5c4)C3)C2)cc(C)n1] | 90347-90-3 |
| 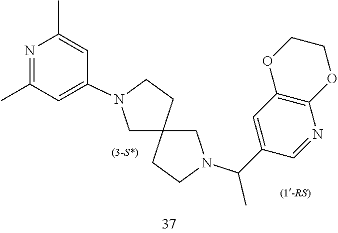 [SMILES: Cc1cc(N2CCC3(CCN(C(C)c4cnc5c(c4)OCCO5)C3)C2)cc(C)n1] | 1254044-15-9 |
| 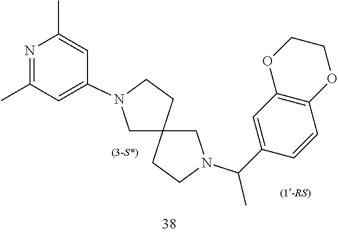 [SMILES: Cc1cc(N2CCC3(CCN(C(C)c4ccc5c(c4)OCCO5)C3)C2)cc(C)n1] | 2879-20-1 |

Preparation of Products 30-32.

The following compounds were prepared following a reductive amination procedure like the one described for the preparation of product 16 starting from 4-chloro-2,6-dimethylpyrimidine and the corresponding amine intermediate using DIPEA in 1,4-dioxane. After isolation of the corresponding compounds these were transformed into the HCl salts by treatment with HCl (4N in 1,4-dioxane). For products 30 and 31 the reaction was run in a 3/1 mixture of 1,4-dioxane/DMF. After isolation of the corresponding products these were transformed into the HCl salts by treatment with HCl (4N in 1,4-dioxane).

TABLE 8

| PRODUCT | INTERMEDIATE AMINE |
|---|---|
| 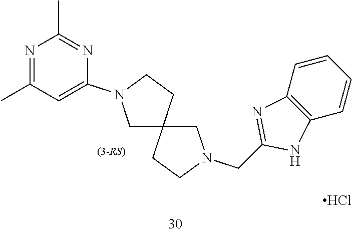 [SMILES: Cc1cc(N2CCC3(CCN(Cc4nc5ccccc5[nH]4)C3)C2)nc(C)n1 ; Cl] | 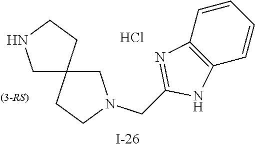 |
| 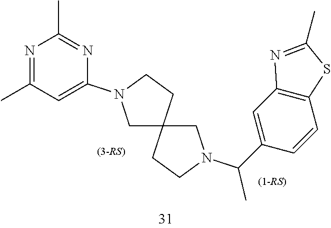 [SMILES: Cc1cc(N2CCC3(CCN(C(C)c4ccc5sc(C)nc5c4)C3)C2)nc(C)n1] | 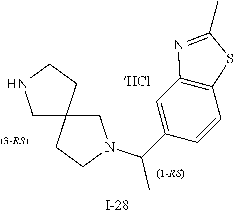 |
| 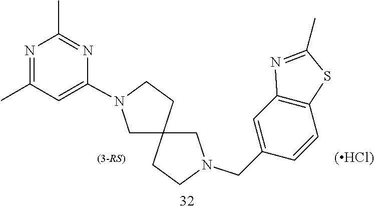 | 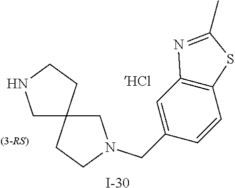 |

Preparation of Products 41 and 42.

Product 40 (175 mg) was subjected to chiral SFC (stationary phase: CHIRALPAK® AD-H 5 μm 250*30 mm, mobile phase: 50% CO₂, 50% EtOH (0.3% iPrNH₂)) yielding product 41 (77 mg) and product 42 (80 mg).

Preparation of Products 47 and 48.

Product 46 (90 mg) was subjected to chiral SFC (stationary phase: Lux Cellulose-2 5 μm 250*21.2 mm, mobile phase: 60% CO₂, 40% EtOH (0.3% iPrNH₂)) yielding product 47 (42 mg) and product 48 (40 mg).

Table 9 provides a summary of all compounds prepared following the methods exemplified in the Experimental Part. In case no salt form is indicated, the compound was obtained as a free base. ‘Exp. No.’ refers to the Example number according to which protocol the compound was synthesized. ‘Co. No.’ means compound number.

TABLE 9

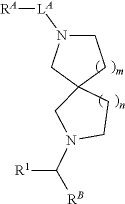

| Co. No. | Exp No. | m | n | L^{A} | R^{A} | R¹ | R^{B} | Stereochem |
|---|---|---|---|---|---|---|---|---|
| 1 | E1 | 1 | 1 | bond | 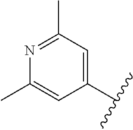 [SMILES: *c1cc(C)nc(C)c1] | H | b-1 (Z¹ = S; Z² = CH; R² = H, R³ = CH₃) | 3-RS |
| 2 | E1 | 1 | 1 | bond | 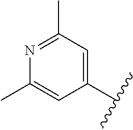 [SMILES: *c1cc(C)nc(C)c1] | H | b-1 (Z¹ = S; Z² = CH; R² = H, R³ = CH₃) | 3-R* |
| 3 | E1 | 1 | 1 | bond | 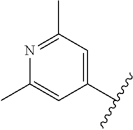 [SMILES: *c1cc(C)nc(C)c1] | H | b-1 (Z¹ = S; Z² = CH; R² = H, R³ = CH₃) | 3-S* |
| 4 | E1 | 1 | 1 | bond | 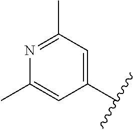 [SMILES: *c1cc(C)nc(C)c1] | H | b-8 | 3-R* |
| 5 | E1 | 1 | 1 | bond | 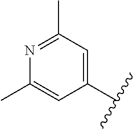 [SMILES: *c1cc(C)nc(C)c1] | H | b-8 | 3-S* |
| 6 | E1 | 1 | 1 | bond | 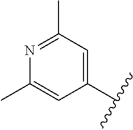 [SMILES: *c1cc(C)nc(C)c1] | CH₃ | b-8 | 3-R*, 1′-RS |
| 7 | E1 | 1 | 1 | bond | 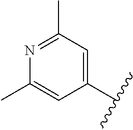 [SMILES: *c1cc(C)nc(C)c1] | CH₃ | b-8 | 3-S*, 1′-RS |
| 8 | E1 | 1 | 1 | bond | 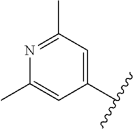 [SMILES: *c1cc(C)nc(C)c1] | CH₃ | b-2 | 3-RS, 1′-RS |
| 9 | E1 | 1 | 1 | bond | 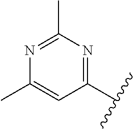 [SMILES: *c1cc(C)nc(C)n1] | H | b-1 (Z¹ = S; Z² = CH; R² = H, R³ = CH₃) | 3-RS |
| 10 | E1 | 1 | 1 | bond | 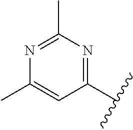 [SMILES: *c1cc(C)nc(C)n1] | CH₃ | b-2 | 3-RS, 1′-RS |
| 11 | E1 | 1 | 1 | bond | 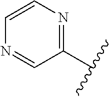 [SMILES: *c1cnccn1] | H | b-1 (Z¹ = S; Z² = CH; R² = H, R³ = CH₃) | 3-RS |
| 12 | E1 | 1 | 1 | bond | 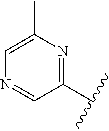 [SMILES: *c1cncc(C)n1] | H | b-1 (Z¹ = S; Z² = CH; R² = H, R³ = CH₃) | 3-RS |
| 13 | E1 | 1 | 1 | bond | 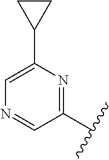 [SMILES: *c1cncc(C2CC2)n1] | H | b-1 (Z¹ = S; Z² = CH; R² = H, R³ = CH₃) | 3-RS |
| 14 | E1 | 1 | 1 | bond | 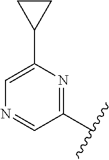 [SMILES: *c1cncc(C2CC2)n1] | H | b-8 | 3-RS |
| 15 | E3 | 1 | 1 | bond | 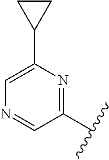 [SMILES: *c1cncc(C2CC2)n1] | CH₃ | b-8 | 3-RS, 1′-RS |
| 16 | E2 | 1 | 1 | bond | 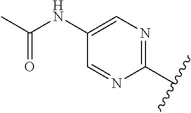 [SMILES: *c1ncc(NC(C)=O)cn1] | CH₃ | b-2 | 3-RS, 1′-RS |
| 17 | E1 | 1 | 1 | CH₂ | 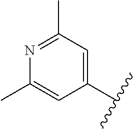 [SMILES: *c1cc(C)nc(C)c1] | H | b-1 (Z¹ = S; Z² = CH; R² = H, R³ = CH₃) | 3-RS |
| 18 | E18 | 1 | 1 | bond | 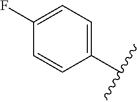 [SMILES: *c1ccc(F)cc1] | H | b-1 (Z¹ = S; Z² = CH; R² = H, R³ = CH₃) | 3-RS |
| 19 | E1 | 1 | 1 | bond | 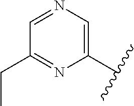 [SMILES: *c1cncc(CC)n1] | H | b-1 (Z¹ = S; Z² = CH; R² = H, R³ = CH₃) | 3-RS |
| 20 | E3 | 1 | 1 | bond | 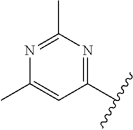 [SMILES: *c1cc(C)nc(C)n1] | CH₃ | b-8 | 3-RS, 1′-RS (•HCl) |
| 21 | E1 | 1 | 1 | bond | 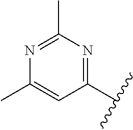 [SMILES: *c1cc(C)nc(C)n1] | H | b-11; R⁵ = H | 3-RS |
| 22 | E1 | 1 | 1 | bond | 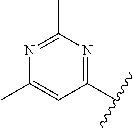 [SMILES: *c1cc(C)nc(C)n1] | H | b-8 | 3-RS (•HCl) |
| 23 | E1 | 0 | 1 | bond | 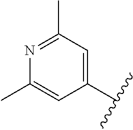 [SMILES: *c1cc(C)nc(C)c1] | H | b-1 (Z¹ = S; Z² = CH; R² = H, R³ = CH₃) | — |
| 24 | E3 | 1 | 1 | bond | 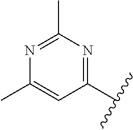 [SMILES: *c1cc(C)nc(C)n1] | CH₃ | b-11; R⁵ = H | 3-RS, 1′-RS (•HCl) |
| 25 | E1 | 1 | 0 | bond | 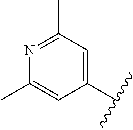 [SMILES: *c1cc(C)nc(C)c1] | H | b-1 (Z¹ = S; Z² = CH; R² = H, R³ = CH₃) | — |
| 26 | E1 | 1 | 1 | bond | 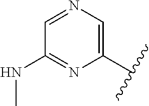 [SMILES: *c1cncc(NC)n1] | H | b-1 (Z¹ = S; Z² = CH; R² = H, R³ = CH₃) | 3-RS |
| 27 | E3 | 1 | 1 | bond | 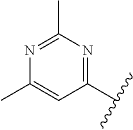 [SMILES: *c1cc(C)nc(C)n1] | CH₃ | b-10; Z³ = CH, R⁴ = CH₃ | 3-R5, 1′-RS (•HCl) |
| 28 | E1 | 1 | 1 | bond | 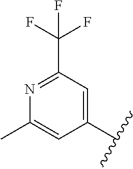 [SMILES: *c1cc(C)nc(C(F)(F)F)c1] | H | b-1 (Z¹ = S; Z² = CH; R² = H, R³ = CH₃) | 3-RS |
| 29 | E1 | 1 | 1 | bond | 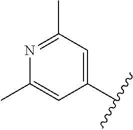 [SMILES: *c1cc(C)nc(C)c1] | CH₃ | b-11; R⁵ = CH₃ | 3-S*, 1′-RS |
| 30 | E2 | 1 | 1 | bond | 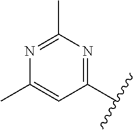 [SMILES: *c1cc(C)nc(C)n1] | H | b-10; Z³ = N, R⁴ = H | 3-RS (•HCl) |
| 31 | E2 | 1 | 1 | bond | 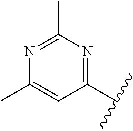 [SMILES: *c1cc(C)nc(C)n1] | CH₃ | b-11; R⁵ = CH₃ | 3-RS, 1′-RS |
| 32 | E2 | 1 | 1 | bond | 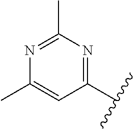 [SMILES: *c1cc(C)nc(C)n1] | H | b-11; R⁵ = CH₃ | 3-RS (•HCl) |
| 33 | E1 | 1 | 1 | bond | 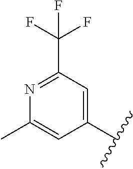 [SMILES: *c1cc(C)nc(C(F)(F)F)c1] | H | b-11; R⁵ = CH₃ | 3-RS |
| 34 | E1 | 1 | 1 | bond | 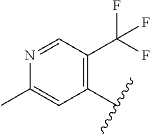 [SMILES: *c1cc(C)ncc1C(F)(F)F] | H | b-1 (Z¹ = S; Z² = CH; R² = H, R³ = CH₃) | 3-RS |
| 35 | E1 | 1 | 1 | CH2 | 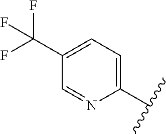 [SMILES: *c1ccc(C(F)(F)F)cn1] | H | b-1 (Z¹ = S; Z² = CH; R² = H, R³ = CH₃) | 3-RS |
| 36 | E1 | 1 | 1 | bond | 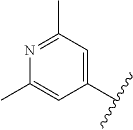 [SMILES: *c1cc(C)nc(C)c1] | CH₃ | b-11; R⁵ = H | 3-S*, 1′-RS |
| 37 | E1 | 1 | 1 | bond | 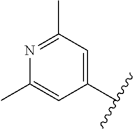 [SMILES: *c1cc(C)nc(C)c1] | CH₃ | b-5 | 3-S*, 1′-RS |
| 38 | E1 | 1 | 1 | bond | 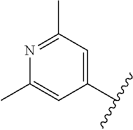 [SMILES: *c1cc(C)nc(C)c1] | CH₃ | b-3 | 3-S*, 1′-RS |
| 39 | E1 | 1 | 1 | bond | 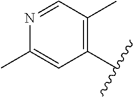 | H | b-1 (Z¹ = S; Z² = CH; R² = H, R³ = CH₃) | 3-RS |
| 40 | E1 | 1 | 1 | bond | 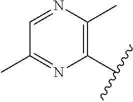 | H | b-1 (Z¹ = S; Z² = CH; R² = H, R³ = CH₃) | 3-RS |
| 41 | E1 | 1 | 1 | bond | 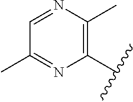 | H | b-1 (Z¹ = S; Z² = CH; R² = H, R³ = CH₃) | 3-R* |
| 42 | E1 | 1 | 1 | bond | 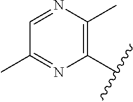 | H | b-1 (Z¹ = S; Z² = CH; R² = H, R³ = CH₃) | 3-S* |
| 43 | E1 | 1 | 1 | bond | 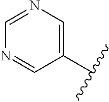 [SMILES: *c1cncnc1] | H | b-1 (Z¹ = S; Z² = CH; R² = H, R³ = CH₃) | 3-RS |
| 44 | E1 | 1 | 1 | bond | 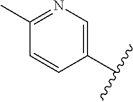 [SMILES: *c1ccc(C)nc1] | H | b-1 (Z¹ = S; Z² = CH; R² = H, R³ = CH₃) | 3-RS |
| 45 | E1 | 1 | 1 | bond | 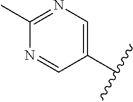 [SMILES: *c1cnc(C)nc1] | H | b-1 (Z¹ = S; Z² = CH; R² = H, R³ = CH₃) | 3-RS |
| 46 | E1 | 1 | 1 | bond | 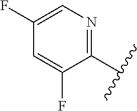 [SMILES: *c1ncc(F)cc1F] | H | b-1 (Z¹ = S; Z² = CH; R² = H, R³ = CH₃) | 3-RS |
| 47 | E1 | 1 | 1 | bond | 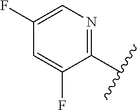 [SMILES: *c1ncc(F)cc1F] | H | b-1 (Z¹ = S; Z² = CH; R² = H, R³ = CH₃) | 3-R* |
| 48 | E1 | 1 | 1 | bond | 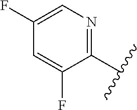 [SMILES: *c1ncc(F)cc1F] | H | b-1 (Z¹ = S; Z² = CH; R² = H, R³ = CH₃) | 3-S* |
| 49 | E1 | 1 | 1 | bond | 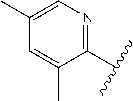 [SMILES: *c1ncc(C)cc1C] | H | b-1 (Z¹ = S; Z² = CH; R² = H, R³ = CH₃) | 3-RS |
| 50 | E1 | 1 | 1 | bond | 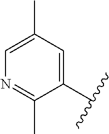 [SMILES: *c1cc(C)cnc1C] | H | b-1 (Z¹ = S; Z² = CH; R² = H, R³ = CH₃) | 3-RS |
| 51 | E1 | 1 | 1 | bond | 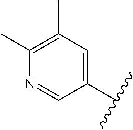 [SMILES: *c1cnc(C)c(C)c1] | H | b-1 (Z¹ = S; Z² = CH; R² = H, R³ = CH₃) | 3-RS |
| 52 | E1 | 1 | 1 | bond | 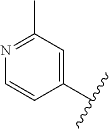 [SMILES: *c1ccnc(C)c1] | H | b-1 (Z¹ = S; Z² = CH; R² = H, R³ = CH₃) | 3-RS |
| 53 | E1 | 1 | 1 | bond | 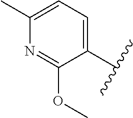 [SMILES: *c1ccc(C)nc1OC] | H | b-1 (Z¹ = S; Z² = CH; R² = H, R³ = CH₃) | 3-RS |
| 54 | E1 | 1 | 1 | bond | 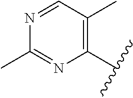 | H | b-1 (Z¹ = S; Z² = CH; R² = H, R³ = CH₃) | 3-RS |
| 55 | E1 | 1 | 1 | bond | 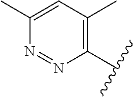 | H | b-1 (Z¹ = S; Z² = CH; R² = H, R³ = CH₃) | 3-RS |
| 56 | E1 | 1 | 1 | bond | 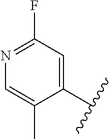 [SMILES: *c1cc(F)ncc1C] | H | b-1 (Z¹ = S; Z² = CH; R² = H, R³ = CH₃) | 3-RS |
| 57 | E1 | 1 | 1 | bond | 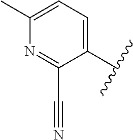 [SMILES: *c1ccc(C)nc1C#N] | H | b-1 (Z¹ = S; Z² = CH; R² = H, R³ = CH₃) | 3-RS |
| 58 | E1 | 1 | 1 | bond | 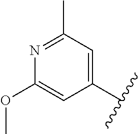 [SMILES: *c1cc(C)nc(OC)c1] | H | b-1 (Z¹ = S; Z² = CH; R² = H, R³ = CH₃) | 3-RS |
| 59 | E1 | 1 | 1 | bond | 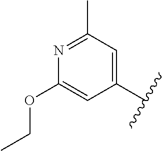 [SMILES: *c1cc(C)nc(OCC)c1] | H | b-1 (Z¹ = S; Z² = CH; R² = H, R³ = CH₃) | 3-RS |
| 60 | E1 | 1 | 1 | bond | 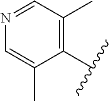 [SMILES: *c1c(C)cncc1C] | H | b-1 (Z¹ = S; Z² = CH; R² = H, R³ = CH₃) | 3-RS |
| 61 | E1 | 1 | 1 | bond | 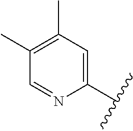 [SMILES: *c1cc(C)c(C)cn1] | H | b-1 (Z¹ = S; Z² = CH; R² = H, R³ = CH₃) | 3-RS |
| 62 | E1 | 1 | 1 | bond | 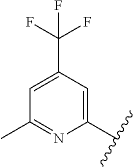 [SMILES: *c1cc(C(F)(F)F)cc(C)n1] | H | b-1 (Z¹ = S; Z² = CH; R² = H, R³ = CH₃) | 3-RS |
| 63 | E1 | 1 | 1 | bond | 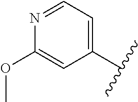 [SMILES: *c1ccnc(OC)c1] | H | b-1 (Z¹ = S; Z² = CH; R² = H, R³ = CH₃) | 3-RS |
| 64 | E1 | 1 | 1 | bond | 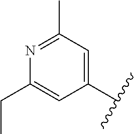 [SMILES: *c1cc(C)nc(CC)c1] | H | b-1 (Z¹ = S; Z² = CH; R² = H, R³ = CH₃) | 3-RS |
| 65 | E1 | 1 | 1 | bond | 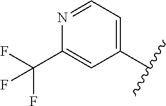 [SMILES: *c1ccnc(C(F)(F)F)c1] | H | b-1 (Z¹ = S; Z² = CH; R² = H, R³ = CH₃) | 3-RS |
| 66 | E1 | 1 | 1 | bond | 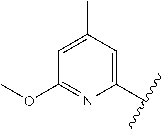 [SMILES: *c1cc(C)cc(OC)n1] | H | b-1 (Z¹ = S; Z² = CH; R² = H, R³ = CH₃) | 3-RS |
| 67 | E1 | 1 | 1 | bond | 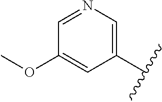 [SMILES: *c1cncc(OC)c1] | H | b-1 (Z¹ = S; Z² = CH; R² = H, R³ = CH₃) | 3-RS |
| 68 | E1 | 1 | 1 | bond | 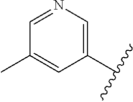 [SMILES: *c1cncc(C)c1] | H | b-1 (Z¹ = S; Z² = CH; R² = H, R³ = CH₃) | 3-RS |
| 69 | E1 | 1 | 1 | bond | 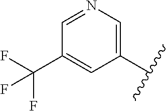 [SMILES: *c1cncc(C(F)(F)F)c1] | H | b-1 (Z¹ = S; Z² = CH; R² = H, R³ = CH₃) | 3-RS |

### C. ANALYTICAL PART

Melting Points

Values are peak values, and are obtained with experimental uncertainties that are commonly associated with this analytical method.

DSC823e (A): For a number of compounds, melting points were determined with a DSC823e (Mettler-Toledo) apparatus. Melting points were measured with a temperature gradient of 10° C./minute. Maximum temperature was 300° C. Values are peak values (A). Mettler Toledo MP50 (B): For a number of compounds, melting points were determined in open capillary tubes on a Mettler MP50 apparatus. Melting points were measured with a temperature gradient of 1, 3, 5 or 10° C./minute. Maximum temperature was 300° C. The melting point was read from a digital display.

LCMS

General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rt) and ions. If not specified differently in the table of data, the reported molecular ion corresponds to the [M+H]⁻ (protonated molecule) and/or [M−H]⁻ (deprotonated molecule). In case the compound was not directly ionizable the type of adduct is specified (i.e. [M+NH₄]⁺, [M+HCOO]⁻, [M+CH₃COO]⁻ etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “QTOF” Quadrupole-Time of Flight, “rt” room temperature, “BEH” bridged ethylsiloxane/silica hybrid, “UPLC” Ultra Performance Liquid Chromatography, “DAD” Diode Array Detector.

TABLE 10
LC-MS Method (Flow expressed in mL/min; column
temperature (T) in ° C.; Run time in min).

| | | | | | FLOW | RUN |
|---|---|---|---|---|---|---|
| METHOD | INSTRUMENT | COLUMN | MOBILE PHASE | GRADIENT | COL T | TIME |
| 1 | Waters: | Waters: | A: 95% | From 95% A | 1 | 5 |
| | Acquity ® | BEH C18 | CH₃COONH₄ | to 5% A in | 50 | |
| | IClass | (1.7 μm, | 6.5 mM + | 4.6 min, held | | |
| | UPLC ®- | 2.1 × 50 mm) | 5% CH₃CN, | for 0.4 min | | |
| | DAD/Xevo | | B: CH₃CN | | | |
| | G2-S | | | | | |
| | QTOF | | | | | |
| 2 | Agilent: | Agilent: | A: 95% | From 95% A | 1 | 7 |
| | HP1100- | Eclipse Plus | CH₃COONH₄ | to 0% A in | 60 | |
| | DAD/ | C18 | 6.5 mM + | 5.0 min, held | | |
| | MSD | (3.5 μm, | 5% CH₃CN, | for 0.15 min, | | |
| | G1956B | 2.1 × 30 mm) | B: CH₃CN | back to 95% A | | |
| | | | | in 0.15 min, | | |
| | | | | held for | | |
| | | | | 1.7 min | | |
| 3 | Waters: | Waters: | A: 95% | From 84.2% A | 0.34 | 6.1 |
| | Acquity | BEH C18 | CH₃COONH₄ | to 10.5% A in | 40 | |
| | UPLC ® | (1.7 μm, | 7 mM + | 2.2 min, held | | |
| | H-Class- | 2.1 × 100 mm) | 5% CH₃CN | for 1.9 min, | | |
| | DAD/SQD2 | | B: CH₃CN | back to 84.2% | | |
| | | | | A in 0.7 min, | | |
| | | | | held for 0.7 | | |
| | | | | min. | | |
| 4 | Waters: | Waters: | A: 95% | 84.2% A for | 0.34 | 6.2 |
| | Acquity | BEH C18 | CH₃COONH₄ | 0.5 min, to | 40 | |
| | UPLC ®- | (1.7 μm, | 7 mM + | 10.5% A in | | |
| | DAD/ | 2.1 × 100 mm) | 5% CH₃CN | 2.2 min, held | | |
| | Quattro | | B: CH₃CN | for 1.9 min, | | |
| | Micro ™ | | | back to 84.2% | | |
| | | | | A in 0.7 min, | | |
| | | | | held for 0.7 | | |
| | | | | min. | | |
| 5 | Agilent | YMC-pack | A: HCOOH | 100% A held | 2.6 | 6.2 |
| | 1100 HPLC | ODS-AQ | 0.1% in H₂O | for 0.2. From | 35 | |
| | DAD | C18 (50 × 4.6 | B: CH₃CN | 100% A to | | |
| | LC/MS | mm, 3 μm) | | 50% A in 4.5 | | |
| | G1956A | | | min, and to | | |
| | | | | 5% A in 0.1 | | |
| | | | | min, held for | | |
| | | | | 1.0 min, to | | |
| | | | | 95% A in 0.2 | | |
| | | | | min. | | |
| 6 | Agilent | YMC-pack | A: HCOOH | From 95% A | 2.6 | 6.2 |
| | 1100 HPLC | ODS-AQ | 0.1% in H₂O | to 5% A in 4.8 | 35 | |
| | DAD | C18 (50 × 4.6 | B: CH₃CN | min, held for | | |
| | LC/MS | mm, 3 μm) | | 1.0 min, to | | |
| | G1956A | | | 95% A in 0.2 | | |
| | | | | min. | | |
| 7 | Agilent | YMC-pack | A: HCOOH | From 95% A | 2.6 | 6.8 |
| | 1260 | ODS-AQ | 0.1% in H₂O | to 5% A in 4.8 | 35 | |
| | Infinity | C18 (50 × | B: CH₃CN | min, held for | | |
| | DAD TOF- | 4.6 mm, 3 | | 1.0 min, to | | |
| | LC/MS | μm) | | 95% A in 0.2 | | |
| | G6224A | | | min. | | |
| 8 | Agilent: | Phenomenex: | A: CF₃CO₂H | 100% A for | 0.8 | 10 |
| | 1200-DAD | Luna-C18 | 0.1% in H₂O, | l min, to 40% | 50 | |
| | and | (2.0 × 50 mm, | B: CF₃CO₂H | A in 4 min, to | | |
| | MSD6110 | 5 μm) | 0.05% in | 15% A in | | |
| | | | CH₃CN | 2.5 min, back | | |
| | | | | to 100% A in | | |
| | | | | 2 min. | | |
| 9 | Agilent: | Waters: | A: CF₃CO₂H | 100% A for | 0.8 | 10 |
| | 1200-DAD | XBridge- | 0.04% in | 1 min, to 40% | 50 | |
| | and | C18 | H₂O, | A in 4 min, to | | |
| | MSD6110 | (2.1 × 50 mm, | B: CF₃CO₂H | 15% A in | | |
| | | 5 μm) | 0.02% in | 2.5 min, back | | |
| | | | CH₃CN | to 100% A in | | |
| | | | | 2 min. | | |

TABLE 11
Analytical data - LCMS: [M + H]⁺ means the protonated mass of the
free base of the compound. Rₜ means retention time (in min). For
some compounds, exact mass was determined.

| Co. | | | | LCMS |
|---|---|---|---|---|
| No. | m.p. | [M + H]⁺ | Rₜ | METHOD |
| 1 | | 386.2 | 1.16 | 1 |
| 2 | | 386.2 | 0.94 | 1 |
| 3 | | 386.2 | 0.95 | 1 |
| 4 | | 374 | 1.13 | 1 |
| 5 | | 374 | 1.09 | 1 |
| 6 | | 388.3 | 1.28 | 1 |
| 7 | | 388 | 1.27 | 1 |
| 8 | | 380.2 | 1.45 | 1 |
| 9 | | 387.2 | 1.04 | 1 |
| 10 | | 381.2 | 1.58 | 1 |
| 11 | | 359 | 1.08 | 1 |
| 12 | | 373.2 | 1.36 | 1 |
| 13 | | 399.2 | 1.95 | 1 |
| 14 | | 387.2 | 2.18 | 1 |
| 15 | | 401 | 2.32/2.38 | 1 |
| 16 | | 410.2 | 1.16 | 1 |
| 17 | | 398.2 | 1.13 | 1 |
| 18 | | 389.3 | 1.06 | 6 |
| 19 | | 387.2 | 1.55 | 1 |
| 20 | 173.1 (B) | 389.3 | 1.06 | 6 |
| 21 | | 380.3 | 1.21 | 7 |
| 22 | | 375.3 | 1.05 | 7 |
| 23 | 212.2 (A) | 372.2 | 0.83 | 1 |
| 24 | 218.2 (B) | 394.2 | 1.17 | 6 |
| 25 | 214.1 (A) | 372.2 | 0.75 | 1 |
| 26 | | 388.2 | 1.23 | 1 |
| 27 | 166.4 (B) | 390.3 | 1.6 | 7 |
| 28 | | 440.2 | 1.81 | 1 |
| 29 | | 407.2 | 1.7 | 1 |
| 30 | | 363.2 | 1.93 | 5 |
| 31 | 230.0 (B) | 408.1 | 2.33 | 5 |
| 32 | 214.8 (B) | 394 | 1.25 | 6 |
| 33 | | 447.2 | 2.48 | 1 |
| 34 | | 440.2 | 1.68 | 1 |
| 35 | | 440 | 2.76 | 2 |
| 36 | | 393.2 | 1.59 | 1 |
| 37 | | 395.2 | 1.18 | 1 |
| 38 | | 394.3 | 1.47 | 1 |
| 39 | | 386.2 | 0.98 | 1 |
| 40 | | 387.2 | 1.41 | 1 |
| 41 | | 387.4 | 2.14 | 3 |
| 42 | | 387.4 | 2.14 | 3 |
| 43 | | 359.2 | 0.92 | 1 |
| 44 | | 372.2 | 1.34 | 1 |
| 45 | | 373.2 | 1 | 1 |
| 46 | | 394.2 | 1.79 | 1 |
| 47 | | 394.2 | 1.78 | 1 |
| 48 | | 394.2 | 1.79 | 1 |
| 49 | | 386.2 | 1.63 | 1 |
| 50 | | 386 | 2.48 | 8 |
| 51 | | 386.1 | 2.54 | 8 |
| 52 | | 372.1 | 1.69 | 4 |
| 53 | | 402 | 3.14 | 8 |
| 54 | | 387 | 2.36 | 8 |
| 55 | | 387 | 2.32 | 8 |
| 56 | | 390 | 2.75 | 8 |
| 57 | | 397 | 3.07 | 8 |
| 58 | | 402 | 2.51 | 8 |
| 59 | | 416.1 | 2.67 | 8 |
| 60 | | 386.1 | 2.39 | 9 |
| 61 | | 386 | 2.56 | 9 |
| 62 | | 440 | 3.12 | 9 |
| 63 | | 388 | 2.94 | 9 |
| 64 | | 400.2 | 2.43 | 9 |
| 65 | | 426 | 2.56 | 9 |
| 66 | | 402 | 2.8 | 9 |
| 67 | | 388 | 2.42 | 9 |
| 68 | | 372 | 2.42 | 9 |
| 69 | | 426 | 3.05 | 9 |

Optical Rotations

Optical rotations were measured on a Perkin-Elmer 341 polarimeter with a sodium lamp and reported as follows: [α]° (λc g/100 ml, solvent, T° C.). [α]_{λ}^{T}=(100α)/(l×c): where l is the path length in dm and c is the concentration in g/100 ml for a sample at a temperature T (° C.) and a wavelength λ (in nm). If the wavelength of light used is 589 nm (the sodium D line), then the symbol D might be used instead. The sign of the rotation (+ or −) should always be given. When using this equation the concentration and solvent are always provided in parentheses after the rotation. The rotation is reported using degrees and no units of concentration are given (it is assumed to be g/100 mL).

TABLE 12
Optical Rotation data.

| | | WAVE- | | | |
|---|---|---|---|---|---|
| Co. | α_{D} | LENGTH | CONCENTRATION | | TEMP. |
| No. | (°) | (nm) | w/v % | SOLVENT | (° C.) |
| 2 | +22.0 | 589 | 0.73 | DMF | 20 |
| 3 | −19.4 | 589 | 0.47 | DMF | 20 |
| I-3a | +11.0 | 589 | 0.93 | DMF | 20 |
| I-3b | −9.5 | 589 | 0.82 | DMF | 20 |
| 4 | +35.6 | 589 | 0.51 | DMF | 20 |
| 5 | −32.4 | 589 | 0.58 | DMF | 20 |
| 41 | +22.2 | 589 | 0.51 | DMF | 20 |
| 42 | −23.1 | 589 | 0.72 | DMF | 20 |
| 48 | +18.7 | 589 | 0.61 | DMF | 20 |

SFCMS-Methods:

General Procedure a for SFC-MS Methods

The SFC measurement was performed using Analytical Supercritical fluid chromatography (SFC) system composed by a binary pump for delivering carbon dioxide (CO₂) and modifier, an autosampler, a columns oven with switching valve for column heating from room temperature to 80° C., a diode array detector equipped with a high-pressure flow cell standing up to 400 bars. Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 13
Analytical SFC-MS Methods (Flow expressed in mL/min; column temperature
(T) in ° C.; Backpressure in bars)

| | | | | | RUN |
|---|---|---|---|---|---|
| | | | | FLOW | TIME |
| METHOD | COLUMN | MOBILE PHASE | GRADIENT | T | BPR |
| 1 | Daicel Chiralpak | A: CO₂ | 40% B | 3.5 | 3.0 |
| | AD-3 (100 × | B: EtOH (+0.3% | hold 3 min | 35 | 105 |
| | 4.6 mm, 3 μm) | iPrNH₂) 60/40 | | | |
| 2 | Lux cellulose 2 | A: CO₂ | 40% B | 3.5 | 3.0 |
| | (100 × 4.6 mm, | B: EtOH (+0.3% | hold 3 min | 35 | 105 |
| | 3 μm) | iPrNH₂) 60/40 | | | |
| | Phenomenex | | | | |

TABLE 14
Analytical SFC data - Rₜ means retention time (in minutes), [M + H]⁺
means the protonated mass of the compound, method refers to the
method used for (SFC)MS analysis of
enantiomerically pure compounds.

| | | | | | ISOMER |
|---|---|---|---|---|---|
| | | | | | ELUTION |
| Co. No. | Rₜ | [M + H]⁺ | UV Area % | METHOD | ORDER |
| 41 | 1.71 | 389 | 100 | 1 | A |
| 42 | 2.24 | 389 | 96.35 | 1 | B |
| 47 | 1.37 | 394 | 100 | 2 | C |
| 48 | 1.71 | 394 | 97.93 | 2 | D |

NMR

For a number of compounds, ¹H NMR spectra were recorded on a Bruker DPX-400 spectrometer operating at 400 MHz, and on a Bruker Avance I operating at 500 MHz, using CHLOROFORM-d (deuterated chloroform, CDCl₃) as solvent. Chemical shifts (δ) are reported in parts per million (ppm) relative to tetramethylsilane (TMS), which was used as internal standard.

TABLE 15
¹H NMR results

| Co. | |
|---|---|
| No. | ¹H NMR RESULT |
| 1 | ¹H NMR (400 MHz, CDCl₃) δ ppm 1.77-2.07 (m, 4 H), 2.30 (s, 3 H), 2.40 (s, 6 |
| | H), 2.47 (d, J = 9.0 Hz, 1 H), 2.59 (d, J = 9.0 Hz, 1 H), 2.65 (td, J = 8.6, 6.5 Hz, 1 H), |
| | 2.77 (td, J = 8.4, 6.0 Hz, 1 H), 3.19 (d, J = 9.7 Hz, 1 H), 3.23-3.41 (m, 3 H), 3.78 (s, |
| | 2 H), 6.07 (s, 2 H), 7.19 (s, 1 H), 11.91 (br s, 1 H) |
| 2 | ¹H NMR (500 MHz, CDCl₃) δ ppm 1.75-2.09 (m, 4 H), 2.31 (s, 3 H), 2.40 (s, 6 |
| | H), 2.47 (d, J = 9.0 Hz, 1 H), 2.60 (d, J = 9.0 Hz, 1 H), 2.66 (td, J = 8.6, 6.5 Hz, 1 H), |
| | 2.78 (td, J = 8.5, 5.9 Hz, 1 H), 3.19 (d, J = 9.5 Hz, 1 H), 3.23-3.38 (m, 3 H), |
| | 3.73-3.84 (m, 2 H), 6.08 (s, 2 H), 7.19 (s, 1 H), 12.48 (br s, 1 H) |
| 3 | ¹H NMR (500 MHz, CDCl₃) δ ppm 1.77-2.06 (m, 4 H), 2.31 (s, 3 H), 2.41 (s, 6 |
| | H), 2.47 (d, J = 9.0 Hz, 1 H), 2.60 (d, J = 9.0 Hz, 1 H), 2.62-2.71 (m, 1 H), 2.78 (td, |
| | J = 8.5, 5.9 Hz, 1 H), 3.19 (d, J = 9.5 Hz, 1 H), 3.24-3.39 (m, 3 H), 3.74-3.83 (m, |
| | 2 H), 6.08 (s, 2 H), 7.19 (s, 1 H), 12.56 (br s, 1 H) |
| 4 | ¹H NMR (500 MHz, CDCl₃) δ ppm 1.82-2.08 (m, 4 H), 2.40 (s, 6 H), 2.53 (d, |
| | J = 9.2 Hz, 1 H), 2.63 (d, J = 9.2 Hz, 1 H), 2.69 (td, J = 8.7, 6.4 Hz, 1 H), 2.79 (td, |
| | J = 8.5, 6.1 Hz, 1 H), 3.24 (d, J = 9.5 Hz, 1 H), 3.26-3.40 (m, 3 H), 3.82-3.90 (m, |
| | 2 H), 6.08 (s, 2 H), 7.82 (dd, J = 8.7, 2.0 Hz, 1 H), 8.03 (d, J = 1.2 Hz, 1 H), 8.07 (d, |
| | J = 8.7 Hz, 1 H), 8.80-8.85 (m, 2 H) |
| 5 | ¹H NMR (500 MHz, CDCl₃) δ ppm 1.80-2.09 (m, 4 H), 2.41 (s, 6 H), 2.53 (d, |
| | J = 9.2 Hz, 1 H), 2.63 (d, J = 9.2 Hz, 1 H), 2.69 (td, J = 8.7, 6.4 Hz, 1 H), 2.79 (td, |
| | J = 8.5, 6.1 Hz, 1 H), 3.24 (d, J = 9.5 Hz, 1 H), 3.26-3.39 (m, 3 H), 3.81-3.91 (m, |
| | 2 H), 6.08 (s, 2 H), 7.82 (dd, J = 8.7, 1.7 Hz, 1 H), 8.03 (d, J = 0.9 Hz, 1 H), 8.07 (d, |
| | J = 8.4 Hz, 1 H), 8.80-8.84 (m, 2 H) |
| 6 | ¹H NMR (400 MHz, CDCl₃) δ ppm 1.42-1.48 (m, 3 H), 1.76-2.06 (m, 4 H), |
| | 2.34-2.45 (m, 0.5 H), 2.39 (s, 3 H), 2.40 (s, 3 H), 2.45-2.53 (m, 1 H), |
| | 2.53-2.76 (m, 2 H), 2.86 (td, J = 8.6, 5.4 Hz, 0.5 H), 3.18-3.39 (m, 4 H), 3.51 (q, J = 6.5 Hz, 1 |
| | H), 6.06 (s, 1 H), 6.08 (s, 1 H), 7.85-7.90 (m, 1 H), 8.00 (d, J = 1.8 Hz, 0.5 H), |
| | 8.01 (d, J = 1.8 Hz, 0.5 H), 8.08 (d, J = 8.6 Hz, 0.5 H), 8.08 (d, J = 8.6 Hz, 0.5 H), |
| | 8.79-8.86 (m, 2 H) |
| 7 | ¹H NMR (400 MHz, CDCl₃) δ ppm 1.39-1.51 (m, 3 H), 1.77-2.05 (m, 4 H), |
| | 2.40 (s, 3 H), 2.38-2.44 (m, 0.5 H), 2.41 (s, 3 H), 2.44-2.53 (m, 1 H), |
| | 2.53-2.75 (m, 2 H), 2.86 (td, J = 8.6, 5.4 Hz, 0.5 H), 3.18-3.38 (m, 4 H), 3.51 (q, J = 6.5 Hz, 1 |
| | H), 6.06 (s, 1 H), 6.08 (s, 1 H), 7.85-7.89 (m, 1 H), 8.00 (d, J = 1.6 Hz, 0.5 H), |
| | 8.01 (d, J = 1.8 Hz, 0.5 H), 8.08 (d, J = 8.8 Hz, 0.5 H), 8.08 (d, J = 8.6 Hz, 0.5 H), |
| | 8.77-8.87 (m, 2 H) |
| 8 | ¹H NMR (500 MHz, CDCl₃) δ ppm 1.29-1.34 (m, 3 H), 1.73-2.02 (m, 4 H), |
| | 2.37-2.42 (m, 7 H), 2.43-2.55 (m, 2 H), 2.62-2.69 (m, 0.4 H), 2.79 (td, J = 8.6, |
| | 5.3 Hz, 0.6 H), 3.14 (q, J = 6.5 Hz, 1 H), 3.17-3.36 (m, 4 H), 5.89-5.97 (m, 2 H), |
| | 6.07 (d, J = 3.5 Hz, 2 H), 6.68-6.77 (m, 2 H), 6.85-6.91 (m, 1 H) |
| 9 | ¹H NMR (400 MHz, CDCl₃) δ ppm 1.78-2.08 (m, 4 H), 2.29 (s, 3 H), 2.33 (s, 3 |
| | H), 2.40-2.94 (m, 4 H), 2.49 (s, 3 H), 3.01-3.73 (m, 4 H), 3.78 (s, 2 H), 5.95 (s, |
| | 1 H), 7.19 (s, 1 H), 11.11 (br s, 1 H) |
| 10 | ¹H NMR (500 MHz, CDCl₃) δ ppm 1.29-1.33 (m, 3 H), 1.69-2.06 (m, 4 H), |
| | 2.31 (s, 3 H), 2.34-2.59 (m, 6 H), 2.70 (br s, 1 H), 3.14 (q, J = 6.6 Hz, 1 H), |
| | 3.19-3.78 (m, 4 H), 5.89-5.96 (m, 3H), 6.68-6.77 (m, 2H), 6.87 (d, J = 5.2 Hz, 1 H) |
| 11 | ¹H NMR (500 MHz, CDCl₃) δ ppm 1.81-1.95 (m, 2 H), 1.95-2.03 (m, 1 H), |
| | 2.03-2.11 (m, 1 H), 2.30 (s, 3 H), 2.59 (s, 2 H), 2.73 (t, J = 7.2 Hz, 2 H), 3.39 (d, |
| | J = 10.1 Hz, 1 H), 3.44-3.52 (m, 2 H), 3.52-3.58 (m, 1 H), 3.75-3.84 (m, 2 H), |
| | 7.20 (s, 1 H), 7.77 (d, J = 2.6 Hz, 1 H), 7.85 (d, J = 1.4 Hz, 1 H), 8.01 (dd, J = 2.7, 1.6 Hz, |
| | 1H), 11.78 (br s, 1H) |
| 12 | ¹H NMR (500 MHz, CDCl₃) δ ppm 1.81-2.08 (m, 4 H), 2.31 (s, 3 H), 2.35 (s, 3 |
| | H), 2.55-2.61 (m, 2 H), 2.73 (t, J = 7.1 Hz, 2 H), 3.39 (d, J = 10.1 Hz, 1 H), |
| | 3.42-3.57 (m, 3 H), 3.74-3.84 (m, 2 H), 7.20 (s, 1 H), 7.64 (s, 1 H), 7.65 (s, 1 H), |
| | 12.42 (br s, 1 H) |
| 13 | ¹H NMR (500 MHz, CDCl₃) δ ppm 0.86-0.93 (m, 2 H), 0.98-1.03 (m, 2 H), |
| | 1.79-1.97 (m, 4 H), 1.97-2.05 (m, 1 H), 2.31 (s, 3 H), 2.54 (d, J = 9.2 Hz, 1 H), |
| | 2.58 (d, J = 9.2 Hz, 1 H), 2.68-2.77 (m, 2 H), 3.33 (d, J = 10.1 Hz, 1 H), |
| | 3.39-3.45 (m, 2 H), 3.46-3.53 (m, 1 H), 3.75-3.83 (m, 2 H), 7.20 (s, 1 H), 7.56 (s, 1 H), |
| | 7.71 (s, 1 H), 12.28 (br s, 1 H) |
| 14 | ¹H NMR (400 MHz, CDCl₃) δ ppm 0.85-0.96 (m, 2 H), 0.97-1.06 (m, 2 H), |
| | 1.81-2.10 (m, 5 H), 2.55 (d, J = 9.2 Hz, 1 H), 2.63 (d, J = 9.0 Hz, 1 H), |
| | 2.67-2.82 (m, 2 H), 3.32-3.56 (m, 4 H), 3.79-3.95 (m, 2 H), 7.56 (s, 1 H), 7.70 (s, 1 H), |
| | 7.84 (dd, J = 8.8, 1.8 Hz, 1 H), 8.02 (d, J = 0.9 Hz, 1 H), 8.07 (d, J = 8.6 Hz, 1 H), |
| | 8.79-8.86 (m, 2 H) |
| 15 | ¹H NMR (500 MHz, CDCl₃) δ ppm 0.86-0.94 (m, 2 H), 0.98-1.03 (m, 2 H), |
| | 1.43-1.48 (m, 3 H), 1.78-2.04 (m, 5 H), 2.44 (d, J = 9.2 Hz, 0.6 H), |
| | 2.47-2.54 (m, 0.8 H), 2.53-2.61 (m, 1 H), 2.63 (d, J = 9.2 Hz, 0.6 H), 2.76 (td, J = 8.6, 5.9 Hz, |
| | 0.6 H), 2.84 (td, J = 8.7, 5.5 Hz, 0.4 H), 3.31-3.55 (m, 5 H), 7.54 (s, 0.4 H), |
| | 7.55 (s, 0.6 H), 7.69 (s, 0.4 H), 7.70 (s, 0.6 H), 7.86-7.90 (m, 1 H), 7.99 (d, J = 1.7 Hz, |
| | 0.4 H), 8.00 (d, J = 2.0 Hz, 0.6 H), 8.05-8.10 (m, 1 H), 8.79-8.84 (m, 2 H) |
| 16 | ¹H NMR (400 MHz, CDCl₃) δ ppm 1.30-1.44 (m, 3 H), 1.81-2.04 (m, 4 H), |
| | 2.17 (s, 3 H), 2.54 (br s, 3 H), 2.72-2.85 (m, 0.6 H), 3.06 (q, J = 7.4 Hz, 0.4 H), |
| | 3.22 (br s, 1 H), 3.43-3.67 (m, 4 H), 5.91-5.96 (m, 2 H), 6.69-6.81 (m, 2 H), |
| | 6.92 (br s, 1 H), 7.07 (br s, 1 H), 8.39 (s, 2 H) |
| 17 | ¹H NMR (500 MHz, CDCl₃) δ ppm 1.75-1.94 (m, 4 H), 2.29 (s, 3 H), |
| | 2.39-2.45 (m, 1 H), 2.45-2.70 (m, 13 H), 3.47 (d, J = 13.9 Hz, 1 H), 3.53 (d, J = 13.9 Hz, 1 |
| | H), 3.71-3.80 (m, 2 H), 6.90 (s, 2 H), 7.19 (s, 1 H), 10.99 (br s, 1 H) |

### D. PHARMACOLOGICAL EXAMPLES

1) OGA—Biochemical Assay

The assay is based on the inhibition of the hydrolysis of fluorescein mono-β-D-N-Acetyl-Glucosamine (FM-GlcNAc) (Mariappa et al. 2015, Biochem J 470:255) by the recombinant human Meningioma Expressed Antigen 5 (MGEA5), also referred to as O-GlcNAcase (OGA). The hydrolysis FM-GlcNAc (Marker Gene technologies, cat # M1485) results in the formation of B-D-N-glucosamineacetate and fluorescein. The fluorescence of the latter can be measured at excitation wavelength 485 nm and emission wavelength 538 nm. An increase in enzyme activity results in an increase in fluorescence signal. Full length OGA enzyme was purchased at OriGene (cat # TP322411). The enzyme was stored in 25 mM Tris.HCl, pH 7.3, 100 mM glycine, 10% glycerol at −20° C. Thiamet G and GlcNAcStatin were tested as reference compounds (Yuzwa et al. 2008 Nature Chemical Biology 4:483; Yuzwa et al. 2012 Nature Chemical Biology 8:393). The assay was performed in 200 mM Citrate/phosphate buffer supplemented with 0.005% Tween-20. 35.6 g Na₂HPO₄ 2 H₂O (Sigma, # C0759) were dissolved in 1 L water to obtain a 200 mM solution. 19.2 g citric acid (Merck, # 1.06580) was dissolved in 1 L water to obtain a 100 mM solution. pH of the sodium phosphate solution was adjusted with the citric acid solution to 7.2. The buffer to stop the reaction consists of a 500 mM Carbonate buffer, pH 11.0. 734 mg FM-GlcNAc were dissolved in 5.48 mL DMSO to obtain a 250 mM solution and was stored at −20° C. OGA was used at 2 nM concentration and FM-GlcNAc at a 100 uM final concentration. Dilutions were prepared in assay buffer.

50 nl of a compound dissolved in DMSO was dispensed on Black Proxiplate™ 384 Plus Assay plates (Perkin Elmer, #6008269) and 3 μl fl-OGA enzyme mix added subsequently. Plates were pre-incubated for 60 min at room temperature and then 2 μl FM-GlcNAc substrate mix added. Final DMSO concentrations did not exceed 1%. Plates were briefly centrifuged for 1 min at 1000rpm and incubate at room temperature for 6 h. To stop the reaction 5 μl STOP buffer were added and plates centrifuge again 1 min at 1000 rpm.

Fluorescence was quantified in the Thermo Scientific Fluoroskan Ascent or the PerkinElmer EnVision with excitation wavelength 485 nm and emission wavelength 538 nm.

For analysis a best-fit curve is fitted by a minimum sum of squares method. From this an IC₅₀ value and Hill coefficient was obtained. High control (no inhibitor) and low control (saturating concentrations of standard inhibitor) were used to define the minimum and maximum values.

2) OGA—Cellular Assay

HEK293 cells inducible for P301L mutant human Tau (isoform 2N4R) were established at Janssen. Thiamet-G was used for both plate validation (high control) and as reference compound (reference EC₅₀ assay validation). OGA inhibition is evaluated through the immunocytochemical (ICC) detection of O-GlcNAcylated proteins by the use of a monoclonal antibody (CTD110.6; Cell Signaling, #9875) detecting O-GlcNAcylated residues as previously described (Dorfmueller et al. 2010 Chemistry & biology, 17:1250). Inhibition of OGA will result in an increase of O-GlcNAcylated protein levels resulting in an increased signal in the experiment. Cell nuclei are stained with Hoechst to give a cell culture quality control and a rough estimate of immediate compounds toxicity, if any. ICC pictures are imaged with a Perkin Elmer Opera Phenix plate microscope and quantified with the provided software Perkin Elmer Harmony 4.1.

Cells were propagated in DMEM high Glucose (Sigma, #D5796) following standard procedures. 2 days before the cell assay cells are split, counted and seeded in Poly-D-Lysine (PDL) coated 96-wells (Greiner, #655946) plate at a cell density of 12,000 cells per cm² (4,000 cells per well) in 100 82 l of Assay Medium (Low Glucose medium is used to reduce basal levels of GlcNAcylation) (Park et al. 2014 The Journal of biological chemistry 289:13519). At the day of compound test medium from assay plates was removed and replenished with 90 μl of fresh Assay Medium. 10 μl of compounds at a 10 fold final concentration were added to the wells. Plates were centrifuged shortly before incubation in the cell incubator for 6 hours. DMSO concentration was set to 0.2%. Medium is discarded by applying vacuum. For staining of cells medium was removed and cells washed once with 100 μl D-PBS (Sigma, #D8537). From next step onwards unless other stated assay volume was always 50 μl and incubation was performed without agitation and at room temperature. Cells were fixed in 50 μl of a 4% paraformaldehyde (PFA, Alpha aesar, # 043368) PBS solution for 15 minutes at room temperature. The PFA PBS solution was then discarded and cells washed once in 10 mM Tris Buffer (LifeTechnologies, # 15567-027), 150 mM NaCl (LifeTechnologies, #24740-0110, 0.1% Triton X (Alpha aesar, # A16046), pH 7.5 (ICC buffer) before being permeabilized in same buffer for 10 minutes. Samples are subsequently blocked in ICC containing 5% goat serum (Sigma, #G9023) for 45-60 minutes at room temperature. Samples were then incubated with primary antibody ( 1/1000 from commercial provider, see above) at 4° C. overnight and subsequently washed 3 times for 5 minutes in ICC buffer. Samples were incubated with secondary fluorescent antibody ( 1/500 dilution, Lifetechnologies, #A-21042) and nuclei stained with Hoechst 33342 at a final concentration of 1 μg/ml in ICC (Lifetechnologies, # H3570) for 1 hour. Before analysis samples were washed 2 times manually for 5 minutes in ICC base buffer.

Imaging is performed using Perkin Elmer Phenix Opera using a water 20× objective and recording 9 fields per well. Intensity readout at 488 nm is used as a measure of O-GlcNAcylation level of total proteins in wells. To assess potential toxicity of compounds nuclei were counted using the Hoechst staining. IC₅₀-values are calculated using parametric non-linear regression model fitting. As a maximum inhibition Thiamet G at a 200 uM concentration is present on each plate. In addition, a concentration response of Thiamet G is calculated on each plate.

TABLE 16
Results in the biochemical and cellular assays.

| | ENZYMATIC | | CELLULAR | |
|---|---|---|---|---|
| Co. | hOGA; | ENZYMATIC | hOGA; | CELLULAR |
| No. | pIC₅₀ | Eₘₐₓ (%) | pEC₅₀ | Eₘₐₓ (%) |
| 1 | 8.22 | 101.9 | 6.85 | 122.7 |
| 2 | 7.99 | 100.7 | n.t. | n.t. |
| 3 | 8.43 | 100.9 | 6.55 | 100.5 |
| 4 | 6.65 | 98.8 | <5 | 23.4 |
| 5 | 6.66 | 98.6 | n.t. | n.t. |
| 6 | 7.01 | 97.8 | n.t. | n.t. |
| 7 | 7.14 | 98.6 | 5.42 | 62.9 |
| 8 | 7.00 | 96.6 | n.t. | n.t. |
| 9 | 7.88 | 100.9 | 6.77 | 85.4 |
| 10 | 6.87 | 99.8 | n.t. | n.t. |
| 11 | 7.09 | 99.1 | n.t. | n.t. |
| 12 | 7.46 | 100.9 | n.t. | n.t. |
| 13 | 7.69 | 100.1 | 6.00 | 92.3 |
| 14 | 6.14 | 94.2 | n.t. | n.t. |
| 15 | 6.64 | 93.4 | n.t. | n.t. |
| 16 | 5.57 | 79 | <5 | 19.5 |
| 17 | 6.25 | 90.6 | n.t. | n.t. |
| 18 | 6.33⁽*⁾ | 93.2 | 5.72 | 86.6 |
| 19 | 6.93 | 102.0 | 6.06 | 56.4 |
| 20 | 6.93 | 97.6 | <3 | 14.9 |
| 21 | 6.77 | 98.1 | <6 | 23.6 |
| 22 | 6.56 | 96.0 | <6 | 12.0 |
| 23 | 6.52 | 100.4 | n.t. | n.t. |
| 24 | 7.31 | 99.3 | <6 | 38.0 |
| 25 | 5.06 | 57.9 | n.t. | n.t. |
| 26 | 7.21 | 101.0 | <6 | 25.1 |
| 27 | 5.58 | 79.4 | n.t. | n.t. |
| 28 | 7.72 | 101.0 | 6.42 | 73.4 |
| 29 | 7.74 | 100.8 | 6.42 | 74.1 |
| 30 | 6.49 | 100.1 | <6 | 7.7 |
| 31 | 7.47 | 101.5 | <6 | 23.7 |
| 32 | 7.29 | 101.5 | <6 | 26.3 |
| 33 | 7.16 | 96.2 | <6 | 6.9 |
| 34 | 7.44 | 102.3 | 6.83 | 85.3 |
| 35 | 5.70 | 84.4 | n.t. | n.t. |
| 36 | 7.62 | 101.4 | 6.70 | 87.9 |
| 37 | 6.74 | 99.0 | <6 | 20.4 |
| 38 | 7.52 | 101.2 | 6.41 | 68.9 |
| 39 | 7.87 | 100.7 | 7.11 | 87.3 |
| 40 | 7.18 | 98.4 | n.t. | n.t. |
| 41 | 7.10 | 99.6 | 6.09 | 52.0 |
| 42 | 6.97 | 100.5 | <6 | 40.3 |
| 43 | 7.27 | 99.8 | n.t. | n.t. |
| 44 | 7.47 | 101.8 | n.t. | n.t. |
| 45 | 7.21 | 99.2 | n.t. | n.t. |
| 46 | 6.95 | 99.0 | n.t. | n.t. |
| 47 | 7.37 | 97.2 | <6 | 43.4 |
| 48 | 6.88 | 99.1 | n.t. | n.t. |
| 49 | 8.41 | 99.6 | 7.34 | 88.8 |
| 50 | 7.60 | 101.0 | 6.73 | 87.8 |
| 51 | 8.07 | 100.2 | 6.91 | 93.0 |
| 52 | 7.79 | 100.5 | 6.68 | 73.7 |
| 53 | 6.51 | 97.1 | n.t. | n.t. |
| 54 | 7.55 | 100.8 | n.t. | n.t. |
| 55 | 7.34 | 99.0 | n.t. | n.t. |
| 56 | 7.29 | 100.0 | n.t. | n.t. |
| 57 | 7.58 | 101.1 | 6.10 | 56.1 |
| 58 | 8.23 | 102.3 | 7.25 | 86.3 |
| 59 | 8.44 | 100.2 | 7.40 | 84.4 |
| 60 | 7.28 | 100.3 | 6.83 | 88.9 |
| 61 | 7.55 | 101.9 | 6.93 | 94.6 |
| 62 | 7.37 | 100.0 | <6 | 22.2 |
| 63 | 8.16 | 101.9 | 6.66 | 75.1 |
| 64 | 8.04 | 101.1 | 6.60 | 62.6 |
| 65 | 7.58 | 100.8 | 6.14 | 56.8 |
| 66 | 7.16 | 99.7 | <6 | 38.5 |
| 67 | 7.46 | 99.4 | 6.27 | 45.6 |
| 68 | 7.99 | 102.8 | 6.86 | 92.9 |
| 69 | 7.31 | 99.1 | 6.17 | 57.6 |

⁽*⁾OGA was used at 10 nM concentration and FM-GlcNAc at a 100 uM final concentration.

1. A compound of Formula (I) 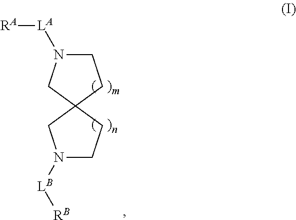
or a stereoisomeric form thereof, wherein
m and n each independently represent 0 or 1, with the proviso that they are not both simultaneously 0;
L^{A} is a covalent bond or CHR; wherein
R is hydrogen or C₁₋₄alkyl optionally substituted with 1, 2 or 3 independently selected halo substituents;
R^{A} represents a 6-membered aryl or heteroaryl radical selected from the group consisting of phenyl, pyridin-2-yl, pyridin-3-yl, pyridin-4-yl, pyridazin-3-yl, pyrimidin-2-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2 or 3 independently selected halo substituents; C₃₋₇cycloalkyl; C₁₋₄alkyloxy optionally substituted with 1, 2 or 3 independently selected halo substituents; and NR^{a}R^{aa}, wherein R^{a} is hydrogen or C₁₋₄alkyl optionally substituted with 1, 2, or 3 independently selected halo substituents, and R^{aa} is selected from the group consisting of hydrogen, C₁₋₄alkyl optionally substituted with 1, 2, or 3 independently selected halo substituents, and
—C(═O)C₁₋₄alkyl;
L^{B} is CHR¹; wherein R¹ is hydrogen or C₁₋₄alkyl optionally substituted with 1, 2 or 3 independently selected halo substituents; and
R^{B} represents a heterocyclic ring or ring system selected from the group consisting of (b-1), (b-2), (b-3), (b-4), (b-5), (b-6), (b-7), (b-8), (b-9), (b-10), (b-11) and (b-12): 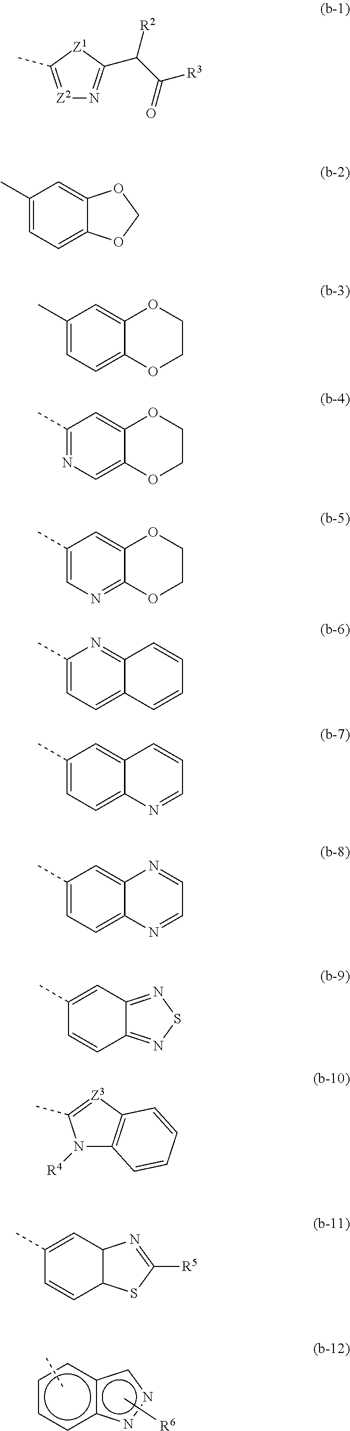
wherein
Z¹ is O, NR^{1z} or S; wherein R^{1z} is hydrogen or C₁₋₄alkyl;
Z² and Z³ each independently represent CH or N;
R³ is C₁₋₄alkyl;
R², R⁴, R⁵ and R⁶ each independently represent hydrogen or C₁₋₄alkyl; or
-L^{B}-R^{B} is a radical of formula (b-13) 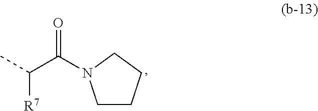
wherein R⁷ is hydrogen or C₁₋₄alkyl;
or a pharmaceutically acceptable addition salt or a solvate thereof.

2. The compound according to claim 1, wherein m is 1 and n is 0 or 1.

3. The compound according to claim 1, wherein L^{B} is CH₂ or CH(CH₃) and R^{B} is a radical of formula (b-1), (b-2), (b-3), (b-8), (b-11) or (b-12).

4. The compound according to claim 1, wherein L^{B} is CH₂ or CH(CH₃) and R^{B} is a radical of formula (b-1) or (b-8).

5. The compound according to claim 1, wherein L^{B} is CH₂ or CH(CH₃) and R^{B} is a radical of formula (b-1), wherein Z¹ is O, Z² is CH, R³ is C₁₋₄alkyl and R² is hydrogen.

6. The compound according to claim 1, wherein R^{A} is pyridin-4-yl, pyrimidin-4-yl or pyrazin-2-yl each of which is optionally substituted with 1 or 2 substituents each independently selected from the group consisting of C₁₋₄alkyl and C₃₋₇cycloalkyl, and all other variables are as defined in any one of claims 1 to 5.

7. The compound according to claim 1, wherein L^{A} is a bond.

8. A pharmaceutical composition comprising a prophylactically or a therapeutically effective amount of a compound according to claim 1 and a pharmaceutically acceptable carrier.

9. (canceled)

10. (canceled)

11. (canceled)

12. A method of preventing or treating a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and argyrophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of a compound according to claim 1.

13. A method for inhibiting O-GlcNAc hydrolase, comprising administering to a subject in need thereof, a prophylactically or a therapeutically effective amount of a compound according to claim 1.

14. A compound of Formula (II) 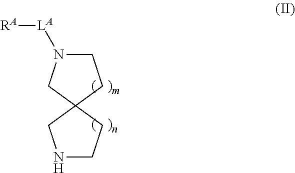
or a stereoisomeric form thereof, wherein
m and n each independently represent 0 or 1, with the proviso that they are not both simultaneously 0;
L^{A} is a covalent bond or CHR; wherein
R is hydrogen or C₁₋₄alkyl optionally substituted with 1, 2 or 3 independently selected halo substituents; and
R^{A} represents a 6-membered aryl or heteroaryl radical selected from the group consisting of phenyl, pyridin-2-yl, pyridin-3-yl, pyridin-4-yl, pyridazin-3-yl, pyrimidin-2-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2 or 3 independently selected halo substituents; C₃₋₇cycloalkyl; C₁₋₄alkyloxy optionally substituted with 1, 2 or 3 independently selected halo substituents; and NR^{a}R^{aa}, wherein R^{a} is hydrogen or C₁₋₄alkyl optionally substituted with 1, 2, or 3 independently selected halo substituents, and R^{aa} is selected from the group consisting of hydrogen, C₁₋₄alkyl optionally substituted with 1, 2, or 3 independently selected halo substituents, and
—C(═O)C₁₋₄alkyl;
or a pharmaceutically acceptable addition salt or a solvate thereof, for use as an OGA inhibitor.
